(19) United States
(12) Patent Application Publication
(10) Pub. No.: US 2010/0120627 A1
(43) Pub. Date: May 13, 2010

(54) GENEMAP OF THE HUMAN GENES ASSOCIATED WITH PSORIASIS

(76) Inventors: Abdelmajid Belouchi, Beaconsfield (CA); John Verner Raelson, Hudson Heights (CA); Walter Edward Bradley, Montreal (CA); Bruno Paquian, Chateauguay (CA); Quynh Nguyen-Huu, Longueuil (CA); Pascal Croteau, Laval (CA); Rene Allard, Montreal (CA); Randall David Little, Dorothee (CA); Johanne Cousineau, Beaconsfield (CA); Sophie Debrus, Montreal (CA); Tim Keith, Bedford, MA (US); Natali Henderson, Pointe-Claire (CA); Daniel Dubois, Laval (CA); Paul Van Eerdewegh, Carlisle, MA (US); Jonathan Segal, Efrat, IL (US)

Correspondence Address: DOWELL & DOWELL P.C., 103 Oronoco St., Suite 220, Alexandria, VA, 22314, US

(21) Appl. No.: 12/309,878

(22) PCT Filed: Aug. 2, 2006
(86) PCT No.: PCT/US06/30281

§ 371 Date: Nov. 18, 2009

(51) Int. Cl. C40B 30/04; C12Q 1/68; C12Q 1/02; C12N 5/02

(52) U.S. Cl. 506/9; 435/6; 435/29; 435/375

(57) ABSTRACT

The present invention relates to the selection of a set of polymorphism markers for use in genome wide association studies based on linkage disequilibrium mapping. In particular, the invention relates to the fields of pharmacogenomics, diagnostics, patient therapy and the use of genetic haplotype information to predict an individual's susceptibility to psoriasis disease and/or their response to a particular drug or drugs.

### FIELD OF THE INVENTION

The invention relates to the field of genomics and genetics, including genome analysis and the study of DNA variations. In particular, the invention relates to the fields of pharmacogenomics, diagnostics, patient therapy and the use of genetic haplotype information to predict an individual's susceptibility to psoriasis disease and/or their response to a particular drug or drugs, so that drugs tailored to genetic differences of population groups may be developed and/or administered to the appropriate population.

The invention also relates to a GeneMap for psoriasis disease, which links variations in DNA (including both genic and non-genic regions) to an individual's susceptibility to psoriasis and/or response to a particular drug or drugs. The invention further relates to the genes disclosed in the GeneMap (see Tables 10, 11 and 12), and methods and reagents for detection of an individual's increased or decreased risk for psoriasis by identifying at least one polymorphism in one or a combination of the genes from the GeneMap. Also related are the candidate regions identified in Table 1, which are associated with psoriasis. In addition the invention further relates to nucleotide sequences of those genes as well as sequences derived therefrom, including isolated genomic DNA and RNA sequences and fragments thereof, cDNA sequences, oligonucleotide probes, single nucleotide polymorphisms (SNPs), alleles and haplotypes (see Sequence Listing and Tables 2-14).

The invention further relates to isolated nucleic acids comprising these nucleotide sequences and isolated polypeptides or peptides encoded thereby. Also related are expression vectors and host cells comprising the disclosed nucleic acids or fragments thereof, as well as antibodies that bind to the encoded polypeptides or peptides.

The present invention further relates to ligands that modulate the activity of the disclosed genes or gene products. In addition, the invention relates to diagnostics and therapeutics for psoriasis disease, utilizing the disclosed nucleic acids, SNPs, chromosomal regions, gene maps, polypeptides or peptides, antibodies and/or ligands and small molecules that activate or repress relevant signaling events.

### BACKGROUND OF THE INVENTION

Inflammatory or allergic skin diseases, especially the proliferative ones, have always been a source of physical and psychological problems for humans and other animals. For some of these skin diseases, such as psoriasis, there is no cure. Psoriasis is a chronic and recurring disease recognized by its raised red silvery scaled eruptions and plaques of various sizes. These plaques can appear anywhere on the skin. Psoriasis is a very visible skin condition that has a high impact on the quality of life of the patient. The scaling is caused by an increase and an abnormally high production of cutaneous cells. The cause of this accelerated cellular growth is unknown, but it is believed that immunological mechanisms may play an important role. The disease is common, affecting from 2 to 4% of the Caucasian population. There are very distinct degrees of psoriasis, regarding the intensity of the cutaneous disorder and the extent and location of the affected areas.

No known therapeutic method can cure psoriasis, but the majority of cases can be controlled. In the less severe cases of the disease, treatment with pomades or emollient creams that keep the skin hydrated can be sufficient. In the more severe cases, the antineoplastics methotrexate or cyclosporin, both of which provoke serious side effects, can be used. In many of the moderate cases of psoriasis, however, topical formulations (pomades, creams, gels and lotions) containing corticosteroids are used by applying them underneath an occlusive covering made of cellophane or polyethylene, or incorporating them into an adhesive bandage. Depending on the affected area, applying these topical formulations can represent a real practical problem for the patient, especially during the day. In addition to the problems inherent in their topical application, another problem with corticosteroids is that the disease does not always respond to treatment and, when it does, it tends to relapse rapidly. The frequency of relapses is relevant to the treatment of psoriasis because the prolonged application of topical corticosteroids causes local side effects (atrophic alterations, loss of collagen, stretch marks, hypertrichosis, telangiectasia and pigmentary disorders) and loss of efficacy.

Despite a preponderance of evidence showing inheritance of a risk for psoriasis through epidemiological studies and genome wide linkage analyses, the genes affecting psoriasis have yet to be discovered. There is a need in the art for identifying specific genes related to psoriasis disease to enable the development of therapeutics that address the causes of the disease rather than relieving its symptoms. The failure in past studies to identify causative genes in complex diseases, such as psoriasis, has been due to the lack of appropriate methods to detect a sufficient number of variations in genomic DNA samples (markers), the insufficient quantity of necessary markers available, and the number of needed individuals to enable such a study. The present invention addresses these issues.

The DNA sequences between two human genomes are 99.9% identical. The variations in DNA sequence between individuals can be as an example, deletions of small or large stretches of DNA, insertions of stretches of DNA, variations in the number of repetitive DNA elements in non-coding regions, and changes in single base positions in the genome called “single nucleotide polymorphisms” (SNPs). Human DNA sequence variation accounts for a large fraction of observed differences between individuals, including susceptibility to disease.

Many common diseases, like psoriasis, are complex genetic traits and are believed to involve several disease-genes rather than single genes, as is observed for rare diseases. This makes detection of any particular gene substantially more difficult than in a rare disease, where a single gene mutation that segregates according to a Mendelian inheritance pattern is the causative mutation. Any one of the multiple interacting gene mutations involved in the etiology of a complex disease will impart a lower relative risk for the disease than will the single gene mutation involved in a simple genetic disease. Low relative risk alleles are more difficult to detect and, as a result, the success of positional cloning using linkage mapping that was achieved for simple genetic disease genes has not been repeated for complex diseases.

Several approaches have been proposed to discover and characterize multiple genes in complex genetic traits. These gene discovery methods can be subdivided into hypothesis-free disease association studies and hypothesis-driven candidate gene or region studies. The candidate gene approach relies on the analysis of a gene in patients who have a disease in which the gene is thought to play a role. This approach is limited in utility because it only provides for the investigation of genes with known functions. Although variant sequences of candidate genes may be identified using this approach, it is inherently limited by the fact that variant sequences in other genes that contribute to the phenotype will be necessarily missed when the technique is employed. Genome-wide scans (GWS) have been shown to be efficient in identifying psoriasis susceptibility genes, such as the MHC class 1 HLA molecules identified at the PSORS 1 locus (HLA-B13, B17 and B37, and HLA-Cw6, Cw7 and DR7.). In contrast to the candidate gene approach, a GWS searches throughout the genome without any a priori hypothesis and consequently can identify genes that are not obvious candidates for the disease as well as genes that are relevant candidates for the disease, as well as chromosomal regions that are structurally important where an “associated allele”, a “particular allele of a polymorphic locus”, or the likes can influence the expression of specific genes.

Family-based linkage mapping methods were initially used for disease locus identification. This technique locates genes based on the relatively limited number of genetic recombination events within the families used in the study, and results in large chromosomal regions containing hundreds of genes, any one of which could be the disease-causing gene. Population-based, or linkage disequilibrium (LD) mapping is based on the premise that regions adjacent to a gene of interest are co-transmitted through the generations along with the gene. As a result, LD extends over shorter genetic regions than does linkage (Hewett et al., 2002), and can facilitate detection of genes with lower relative risk than family linkage mapping approaches. It also defines much smaller candidate regions which may contain only a few genes, making the identification of the actual disease gene much easier.

It has been estimated that a genome wide scan that uses a general population and case/control association (LD) analysis would require approximately 700,000 SNP markers (Carlson et al., 2003). The cost of a GWS at this marker density for a sufficient sample size for statistical power is economically prohibitive. The use of a special founder population (genetic isolates), such as the French Canadian population of Quebec, is one solution to the problem with LD analysis. The French Canadian population in Quebec (Quebec Founder Population—QFP) provides one of the best resources in the world for gene discovery based on its high levels of genetic sharing and genetic homogeneity. By combining DNA collected from the QFP, high throughput genotyping capabilities and proprietary algorithms for genetic analysis, a comprehensive genome-wide association study was facilitated. The present invention relates specifically to a set of psoriasis-causing genes (GeneMap) and targets which present attractive points of therapeutic intervention.

Identifying susceptibility genes associated with psoriasis disease and their respective biochemical pathways will facilitate the identification of diagnostic markers as well as novel targets for improved therapeutics. It will also improve the quality of life for those afflicted by this disease and will reduce the economic costs of these afflictions at the individual and societal level. The identification of those genetic markers would provide the basis for novel genetic tests and eliminate or reduce the need for the battery of laboratory and biopsy tests currently used. The present invention satisfies this need and provides related advantages as well.

### BRIEF DESCRIPTION OF THE DRAWINGS

FIGS. 1 to 13: Graphical representation of networks 1 to 13. Lists of directly interacting genes, as described in the text, were imported in the IPA software from Ingenuity Systems Inc. to generate networks of interacting genes. These networks are based on functional relationships between gene products using known interactions in the literature.

### DESCRIPTION OF THE FILES CONTAINED ON THE CD-R

The contents of the submission on compact discs submitted herewith are incorporated herein by reference in their entirety: A compact disc copy of the Sequence Listing (COPY 1) (filename: GENI 017/00WO Sequence Listing.txt, date recorded: Jul. 31, 2006, file size 80,914 kilobytes); a duplicate compact disc copy of the Sequence Listing (COPY 2) (filename: GENI 017/00WO Sequence Listing.txt, date recorded: Jul. 31, 2006, file size 80,914 kilobytes); a duplicate compact disc copy of the Sequence Listing (COPY 3) (filename: GENI 017/00WO Sequence Listing.txt, date recorded: Jul. 31, 2006, file size 80,914 kilobytes); a computer readable format copy of the Sequence Listing (CRF COPY) (filename: GENI 017/00WO Sequence Listing.txt, date recorded: Jul. 31, 2006, file size 80,914 kilobytes); a compact disc copy of the Tables (COPY 1) (filename: GENI 017/00WO Tables.pdf, date recorded: Jul. 31, 2006, file size 15,060 kilobytes); a duplicate compact disc copy of the Tables (COPY 2) (filename: GENI 017/00WO Tables.pdf, date recorded: Jul. 31, 2006, file size 15,060 kilobytes); and a duplicate compact disc copy of the Tables (COPY 3) (filename: GENI 017/00WO Tables.pdf, date recorded: Jul. 31, 2006, file size 15,060 kilobytes).

Definitions

Throughout the description of the present invention, several terms are used that are specific to the science of this field. For the sake of clarity and to avoid any misunderstanding, these definitions are provided to aid in the understanding of the specification and claims:

Allele: One of a pair, or series, of forms of a gene or non-genic region that occur at a given locus in a chromosome. Alleles are represented by variations of the same basic symbol (e.g., B for dominant and b for recessive); B1, B2, Bn for n additive alleles at a locus). In a normal diploid cell there are two alleles of any one gene (one from each parent), which occupy the same relative position (locus) on homologous chromosomes. Within a population there may be more than two alleles of a gene, i.e., multiple alleles. SNPs also have alleles, i.e., the two (or more) nucleotides that characterize the SNP.

Amplification of nucleic acids: refers to methods such as polymerase chain reaction (PCR), ligation amplification (or ligase chain reaction, LCR), amplification methods based on the use of Q-beta replicase, and any other in vitro amplification reaction known in the art or to be developed in the future. These methods are well known in the art and are described, for example, in U.S. Pat. Nos. 4,683,195 and 4,683,202. Reagents and hardware for conducting PCR are commercially available. Primers useful for amplifying sequences from the disorder region are preferably complementary to, and preferably hybridize specifically to, sequences in the disease region, i.e., region associated with psoriasis, or in regions that flank a target region therein. Genes from Tables 10-12 generated by amplification may be sequenced directly. Alternatively, the amplified sequence(s) may be cloned prior to sequence analysis.

Antigenic component: is a moiety that binds to its specific antibody with sufficiently high affinity to form a detectable antigen-antibody complex.

Antibodies: refer to polyclonal and/or monoclonal antibodies and fragments thereof, and immunologic binding equivalents thereof, including Fab fragments (Fab)₂ fragments, Fv fragments, humanized antibodies, chimeric antibodies, etc., that can bind to proteins and fragments thereof or to nucleic acid sequences from the disease region, particularly from the disease gene products or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera are tested for immunoreactivity to the protein or fragment. Monoclonal antibodies may be made by injecting mice with the proteins, or fragments thereof. Monoclonal antibodies will be screened by ELISA and tested for specific immunoreactivity with protein or fragments thereof. (Harlow et al. 1988, Antibodies: A Laboratory Manual, Cold Spring Harbor Laboratory, Cold Spring Harbor, N.Y.). These antibodies will be useful in assays as well as therapeutics.

Associated allele: refers to an allele at a polymorphic locus that is associated with a particular phenotype of interest, e.g., a predisposition to disease or a particular drug response.

cDNA: refers to complementary or copy DNA produced from an RNA template by the action of RNA-dependent DNA polymerase (reverse transcriptase). Thus, a cDNA clone means a duplex DNA sequence complementary to an RNA molecule of interest, included in a cloning vector or PCR amplified. This term includes genes from which intervening sequences, including non-coding sequences, have been removed.

cDNA library: refers to a collection of recombinant DNA molecules containing cDNA inserts that together comprise essentially all of the expressed genes of an organism or tissue. A cDNA library can be prepared by methods known to one skilled in the art (see, e.g., Cowell and Austin, 1997, “DNA Library Protocols,” Methods in Molecular Biology). Generally, RNA is first isolated from the cells of the desired organism, and the RNA is used to prepare cDNA molecules.

Cloning: refers to the use of recombinant DNA techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

Cloning vector: refers to a plasmid or phage DNA or other DNA molecule that is able to replicate in a host cell. The cloning vector is typically characterized by one or more endonuclease recognition sites at which such DNA sequences may be cleaved in a determinable fashion without loss of an essential biological function of the DNA, and which may contain a selectable marker suitable for use in the identification of cells containing the vector.

Coding sequence or a protein-coding sequence: is a polynucleotide sequence capable of being transcribed into mRNA and/or capable of being translated into a polypeptide or peptide. The boundaries of the coding sequence are typically determined by a translation start codon at the 5′-terminus and a translation stop codon at the 3′-terminus.

Complement of a nucleic acid sequence: refers to the antisense sequence that participates in Watson-Crick base-pairing with the original sequence.

Disorder region: refers to the portions of the human chromosomes displayed in Table 1 bounded by the markers from Tables 2-13.

Disorder-associated nucleic acid or polypeptide sequence: refers to a nucleic acid sequence that maps to region of Table 1 or the polypeptides encoded therein (Tables 2-14 SNPs, nucleic acids, and polypeptides). For nucleic acids, this encompasses sequences that are identical or complementary to the gene sequences from Tables 4-6, as well as sequence-conservative, function-conservative, and non-conservative variants thereof. For polypeptides, this encompasses sequences that are identical to the disclosed polypeptides, as well as function-conservative and non-conservative variants thereof. Included are the alleles of naturally-occurring polymorphisms causative of psoriasis such as, but not limited to, alleles that cause altered expression of genes of Tables 10-12 and alleles that cause altered protein levels, conformation or stability (e.g., decreased levels, increased levels, expression in an inappropriate tissue type, increased stability, and decreased stability).

Expression vector: refers to a vehicle or plasmid that is capable of expressing a gene that has been cloned into it, after transformation or integration in a host cell. The cloned gene is usually placed under the control of (i.e., operably linked to) a regulatory sequence.

Function-conservative variants: are those in which a change in one or more nucleotides in a given codon position results in a polypeptide sequence in which a given amino acid residue in the polypeptide has been replaced by a conservative amino acid substitution. Function-conservative variants also include analogs and homologs of a given polypeptide and any polypeptides that have the ability to elicit antibodies specific to a designated polypeptide.

Founder population: Also called a population isolate, this is a large number of people who have mostly descended, in genetic isolation from other populations, from a much smaller number of people who lived many generations ago.

Gene: Refers to a DNA sequence that encodes through its template or messenger RNA a sequence of amino acids characteristic of a specific peptide, polypeptide, or protein. The term “gene” also refers to DNA sequence that encodes an RNA product. The term gene as used herein with reference to genomic DNA includes intervening, non-coding regions, as well as regulatory regions, including regions at one or both of the 5′ and 3′ ends. A gene sequence is wild-type if such sequence is usually found in individuals unaffected by the disease or condition of interest. However, environmental factors and other genes can also play an important role in the ultimate determination of the disease. In the context of complex diseases involving multiple genes (oligogenic diseases), the wild type, or normal sequence can also be associated with a measurable risk or susceptibility, receiving its reference status based on its frequency in the general population.

GeneMaps are defined as groups of gene(s) that are directly or indirectly involved in at least one phenotype of a particular disease or disorder, for instance, psoriasis. As such, GeneMaps enable the development of synergistic diagnostic products, creating “theranostics”.

Genotype: Set of alleles at a specified locus or loci.

Haplotype: The allelic pattern of a group of (usually contiguous) DNA markers or other polymorphic loci along an individual chromosome or double helical DNA segment. Haplotypes identify individual chromosomes or chromosome segments. The presence of shared haplotype patterns among a group of individuals implies that the locus defined by the haplotype has been inherited, identical by descent (IBD), from a common ancestor. Detection of identical by descent haplotypes is the basis of linkage disequilibrium (LD) mapping. Haplotypes are broken down through the generations by recombination and mutation. In some instances, a specific allele or haplotype may be associated with susceptibility to a disease or condition of interest, e.g., psoriasis disease. In other instances, an allele or haplotype may be associated with a decrease in susceptibility to a disease or condition of interest, i.e., a protective sequence.

Host: includes prokaryotes and eukaryotes. The term includes an organism or cell that is the recipient of an expression vector (e.g., autonomously replicating or integrating vector).

Hybridizable: nucleic acids are hybridizable to each other when at least one strand of the nucleic acid can anneal to another nucleic acid strand under defined stringency conditions. In some embodiments, hybridization requires that the two nucleic acids contain a stretch of at least 10 completely complementary or substantially complementary nucleotides; depending on the stringency of hybridization, mismatches may be tolerated. The appropriate stringency for hybridizing nucleic acids depends on the length of the nucleic acids and the degree of complementarily, and can be determined in accordance with the methods described herein.

Identity by descent (IBD): Identity among DNA sequences for different individuals that is due to the fact that they have all been inherited from a common ancestor. LD mapping identifies IBD haplotypes as the likely location of disease genes shared by a group of patients.

Identity or percentage identity: as known in the art, is a relationship between two or more polypeptide sequences or two or more polynucleotide sequences, as determined by comparing the sequences. In the art, identity refers to the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, ComputerAnalysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

Immunogenic component: is a moiety that is capable of eliciting a humoral and/or cellular immune response in a host animal.

Isolated nucleic acids: are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods, and library screening methods.

Isolated polypeptides or peptides: are those that are separated from other components (e.g., DNA, RNA, and other polypeptides or peptides) with which they are associated (e.g., as obtained from cells, translation systems, or chemical synthesis systems). In a preferred embodiment, isolated polypeptides or peptides are at least 10% pure; more preferably, 80% or 90% pure. Isolated polypeptides and peptides include those obtained by methods described herein, or other established methods, including isolation from natural sources (e.g., cells, tissues, or organs), chemical synthesis, recombinant methods, or combinations of recombinant and chemical methods. Proteins or polypeptides referred to herein as recombinant are proteins or polypeptides produced by the expression of recombinant nucleic acids. A portion as used herein with regard to a protein or polypeptide, refers to fragments of that protein or polypeptide. The fragments can range in size from about 5 amino acid residues to all but one residue of the entire protein sequence. Thus, a portion or fragment can be at least about 5 to about 50, about 50 to about 100, about 100 to about 200, about 200 to about 400, about 400 to about 800, or more consecutive amino acid residues of a protein or polypeptide, and more specifically, at least about 5, at least about 10, at least about 15, at least about 20, at least about 25, at least about 30, at least about 50, at least about 75, at least about 100, at least about 150, at least about 200, at least about 300, at least about 500, at least about 800, or more consecutive amino acid residues of a protein or polypeptide.

Linkage disequilibrium (LD): the situation in which the alleles for two or more loci do not occur together in individuals sampled from a population at frequencies predicted by the product of their individual allele frequencies. In other words, markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.

LD mapping: population based gene mapping, which locates disease genes by identifying regions of the genome where haplotypes or marker variation patterns are shared statistically more frequently among disease patients compared to healthy controls. This method is based upon the assumption that many of the patients will have inherited an allele associated with the disease from a common ancestor (IBD), and that this allele will be in LD with the disease gene.

Locus: a specific position along a chromosome or DNA sequence. Depending upon context, a locus could be a gene, a marker, a chromosomal band or a specific sequence of one or more nucleotides.

Minor allele frequency (MAF): the population frequency of one of the alleles for a given polymorphism, which is equal or less than 50%. The sum of the MAF and the Major allele frequency equals one.

Markers: an identifiable DNA sequence that is variable (polymorphic) for different individuals within a population. These sequences facilitate the study of inheritance of a trait or a gene. Such markers are used in mapping the order of genes along chromosomes and in following the inheritance of particular genes; genes closely linked to the marker or in LD with the marker will generally be inherited with it. Two types of markers that are commonly used in genetic analysis include microsatellites and SNPs.

Microsatellite: DNA of eukaryotic cells comprising a repetitive, short sequence of DNA that is present as tandem repeats and in highly variable copy number, flanked by sequences unique to that locus.

Mutant sequence: a sequence that differs from one or more wild-type sequences. For example, a nucleic acid from a gene listed in Tables 10-12 containing a particular allele of a single nucleotide polymorphism may be a mutant sequence. In some cases, the individual carrying this allele has increased susceptibility toward the disease or condition of interest. In other cases, the mutant sequence might also refer to an allele that decreases the susceptibility toward a disease or condition of interest and thus acts in a protective manner. The term mutation may also be used to describe a specific allele of a polymorphic locus.

Non-conservative variants: are those in which a change in one or more nucleotides in a given codon position results in a polypeptide sequence in which a given amino acid residue in a polypeptide has been replaced by a non-conservative amino acid substitution. Non-conservative variants also include polypeptides comprising non-conservative amino acid substitutions.

Nucleic acid or polynucleotide: purine- and pyrimidine-containing polymers of any length, either polyribonucleotides or polydeoxyribonucleotide or mixed polyribo polydeoxyribonucleotides. This includes single- and double-stranded molecules, i.e., DNA-DNA, DNA-RNA and RNA-RNA hybrids, as well as protein nucleic acids (PNA) formed by conjugating bases to an amino acid backbone. This also includes nucleic acids containing modified bases.

Nucleotide: a nucleotide, the unit of a DNA molecule, is composed of a base, a 2′-deoxyribose and phosphate ester(s) attached at the 5′ carbon of the deoxyribose. For its incorporation in DNA, the nucleotide needs to possess three phosphate esters but it is converted into a monoester in the process.

Operably linked: with reference to a gene and a promoter or regulatory sequence means that the promoter or regulatory sequence controls the initiation of or regulates the expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

Ortholog: denotes a gene or polypeptide obtained from one species that has homology to an analogous gene or polypeptide from a different species.

Paralog: denotes a gene or polypeptide obtained from a given species that has homology to a distinct gene or polypeptide from that same species.

Phenotype: any visible, detectable or otherwise measurable property of an organism such as symptoms of or susceptibility to, a disease.

Polymorphism: occurrence of two or more alternative genomic sequences or alleles between or among different genomes or individuals at a single locus. A polymorphic site thus refers specifically to the locus at which the variation occurs. In some cases, an individual carrying a particular allele of a polymorphism has an increased or decreased susceptibility toward a disease or condition of interest.

Portion: with regard to a nucleic acid or polynucleotide, refers to fragments of that nucleic acid or polynucleotide. Accordingly, portion and fragment are synonymous. The fragments can range in size from about 8 nucleotides to all but one nucleotide of the entire gene sequence. Preferably, the fragments are at least about 8 to about 10 nucleotides in length; at least about 12 nucleotides in length; at least about 15 to about 20 nucleotides in length; at least about 25 nucleotides in length; at least about 35 to about 55 nucleotides in length; at least about 75 to about 100 nucleotides in length; at least about 125 to about 150 nucleotides in length; at least about 175 to about 200 nucleotides in length; at least about 200 to about 300 nucleotides in length, or more.

Probe or primer: refers to a nucleic acid or oligonucleotide that forms a hybrid structure with a sequence in a target region of a nucleic acid due to complementarity of the probe or primer sequence to at least one portion of the target region sequence.

Protein: is synonymous with polypeptide. Peptides are defined as fragments or portions of polypeptides, preferably fragments or portions having at least one functional activity (e.g., proteolysis, adhesion, fusion, antigenic, or intracellular activity) of the complete polypeptide sequence.

Psoriasis: a chronic and recurring inflammatory skin disease recognized by its raised red silvery scaled eruptions and plaques of various sizes. Types of psoriasis include psoriasis vulgaris or plaque psoriasis (the most common type), nail psoriasis, scalp psoriasis, pustular psoriasis, guttate psoriasis, inverse or flexural psoriasis, erythrodermic psoriasis and psoriatic arthritis.

Recombinant nucleic acids: nucleic acids which have been produced by recombinant DNA methodology, including those nucleic acids that are generated by procedures which rely upon a method of artificial replication, such as the polymerase chain reaction (PCR) and/or cloning into a vector using restriction enzymes. Portions of recombinant nucleic acids which code for polypeptides can be identified and isolated by, for example, the method of M. Jasin et al., U.S. Pat. No. 4,952,501.

Regulatory sequence: refers to a nucleic acid sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Regulatory sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

Sample: as used herein refers to a biological sample, such as, for example, tissue or fluid isolated from an individual or animal (including, without limitation, plasma, serum, cerebrospinal fluid, lymph, tears, saliva, milk, pus, and tissue exudates and secretions) or from in vitro cell culture-constituents, as well as samples obtained from, for example, a laboratory procedure.

Single nucleotide polymorphism (SNP): variation of a single nucleotide. This includes the replacement of one nucleotide by another and deletion or insertion of a single nucleotide. Typically, SNPs are biallelic markers although tri- and tetra-allelic markers also exist. For example, SNP A\C may comprise allele C or allele A (Tables 2-9 and 13). Thus, a nucleic acid molecule comprising SNP A\C may include a C or A at the polymorphic position. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In some embodiments, the SNPs in a haplotype are in linkage disequilibrium with one another.

Sequence-conservative: refers to variants in which a change of one or more nucleotides in a given codon position results in no alteration in the amino acid encoded at that position (i.e., silent mutation).

Substantially homologous: a nucleic acid or fragment thereof is substantially homologous to another if, when optimally aligned (with appropriate nucleotide insertions and/or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90% (M. Kanehisa, 1984, NucL Acids Res. 11:203-213). The length of homology comparison, as described, may be over longer stretches, and in certain embodiments will often be over a stretch of at least about 14 nucleotides, usually at least about 20 nucleotides, more usually at least about 24 nucleotides, typically at least about 28 nucleotides, more typically at least about 32 nucleotides, and preferably at least about 36 or more nucleotides.

Wild-type gene from Tables 10-12: refers to the reference sequence. The wild-type gene sequences from Tables 10-12 are used to identify the variants (polymorphisms, alleles, and haplotypes) described in detail herein.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Standard reference works setting forth the general principles of recombinant DNA technology include J. Sambrook et a., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes I and 11; M. J. Gait (ed), 1984, Oligonucleotide Synthesis; B. D. Hames and S. J. Higgins (eds), 1984, Nucleic Acid Hybridization; Wu and Grossman (eds), Methods in Enzymology (Academic Press, Inc.), Vol. 154 and Vol. 155; Quirke and Taylor (eds), 1991, PCR-A Practical Approach; Harries and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning, J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology. Tools for Genomic and Molecular Research, American Society for Microbiology, Washington, D.C. Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Int'l Publishers Ltd., Publ., London; D. P. Stites and A. L Terr, 1991, Basic and Clinical Immunology, 7th Ed., Appleton & Lange, Publ., Norwalk, Conn.; and A. K. Abbas et al., 1991, Cellular and Molecular Immunology, W. B. Saunders Co., Publ., Philadelphia, Pa. Any suitable materials and/or methods known to those of skill can be utilized in carrying out the present invention; however, preferred materials and/or methods are described. Materials, reagents, and the like to which reference is made in the following description and examples are generally obtainable from commercial sources, and specific vendors are cited herein.

### DETAILED DESCRIPTION OF THE INVENTION

General Description of Psoriasis Disease

Psoriasis vulgaris is a chronic inflammatory skin disease, the prevalence rate of which is 2-5% in Caucasian populations; 1-2% in English and European populations; and 0.1-0.3% in Far Eastern and Chinese populations. Most cases of psoriasis vulgaris are sporadic. Sporadic cases are characterized by inflammatory skin lesions showing abnormal differentiation and hyperproliferation of keratinocytes, infiltration of activated helper T cells and monocytes, hypervascularization, and release of proinflammatory cytokines.

Psoriasis is a T-cell-mediated inflammatory disease in which the activation of the immune system in focal skin regions, mediated by CD8+ and CD4+ T lymphocytes, results in epidermal hyperplasia (Krueger, 2002). The cause of psoriatic lesions has been suggested to be related to antigens/superantigens or autoantigens provided by non-dermal inducing factors (Freedberg et al., 1998).

Three basic treatments are currently used for psoriasis: 1) applying a topical agent, 2) using phototherapy, and 3) applying systemic agents. Applying a topical agent is typically the first approach to treating psoriasis. Topical agents include corticosteroids, coal tar, anthralin, calcipotriene, and tazarotene. The treatment of choice for providing symptomatic relief entails the topical application of corticosteroids. All topical steroids have anti-inflammatory, anti-pruritic, and vasoconstrictive effects. However, their long-term use is often accompanied by loss of effectiveness.

Topical coal tar contains more than 10,000 different chemical substances. The exact mechanism of action thereof is unknown. The most common coal tar treatment protocol (the Goeckermann method, which also uses UV phototherapy) involves almost a month of messy topical treatments at a day treatment center. Although this method has a high rate of success in clearing skin, it is relatively expensive and time-consuming. Anthralin is a synthetic derivative of a tree bark extract and is a cellular antiproliferative agent that decreases the rate of epidermal cell growth. Although anthralin is considered one of the most effective agents available for treating psoriasis, it is not in widespread use because of its high potential to cause irritation and staining of the skin. Calcipotriene is a synthetic vitamin D-3 analog that regulates skin cell production. It is not in widespread use, however, because it is expensive and dosage is limited due to the risk of irritation and vitamin D toxicity. Tazarotene is a retinoid derivative that has been used to topically treat psoriasis. However, it often causes irritation and, thus, is typically used in conjunction with topical steroid treatments. For plaque psoriasis, retinoids are used in combination with ultraviolet phototherapy to minimize the dosage thereof that is required. The utility of such methods is limited by the side effects and precautions that are generally associated with retinoids, as would be appreciated by one skilled in the art.

Phototherapy is generally used only in the presence of extensive, widespread disease. Resistance to other topical treatments is another indication for phototherapy. There are two main forms of phototherapy, UVB and PUVA phototherapy. UVB, or Ultraviolet B, phototherapy uses light having a wavelength in the range of 290-320 nm. Such phototherapy is usually combined with one or more topical treatments including: topically applying coal tar, followed by using UVB (the aforementioned Goeckerman method); using a coal tar bath, followed by UVB, and then followed by topically applying anthralin (the Ingram method); or using UVB in combination with topically applying corticosteroids, calcipotriene, tazarotene, or simply bland emollients. A major drawback of such methods is the long duration thereof and accessibility to UVB equipment. PUVA uses the photosensitizing drug methoxsalen (8methoxypsoralens) in conjunction with UVA light (wavelengths in the 320-400 nm range). PUVA interferes with DNA synthesis (methoxsalen binds covalently to pyrimidine bases in DNA), decreases cellular proliferation, and induces apoptosis of cutaneous lymphocytes leading to localized immunosuppression. Adverse effects associated with both of these treatments include nausea, pruritus, burning, photo damage to the skin and increased risk of skin cancer. Systemic psoriasis treatment is usually initiated only after both topical treatment and phototherapy have failed, or for patients with very active psoriatic arthritis. The main agents available are the immunomodulators Methotrexate and Cyclosporine, and the oral retinoid Acitretin, as well as and new biological agents. For example, recent new proposed treatments involve recombinant human cytokines, growth factors, or monoclonal antibodies and fusion proteins thereof (Wons et al., 2002). Methotrexate is a folic acid antagonist that inhibits DNA synthesis in tissues with high rates of turnover, such as psoriatic plaques, and is immunosuppresive to mononuclear cells in the skin, blood, and lymphatic system. Methotrexate has toxic effects on hematologic, renal, GI, pulmonary, and neurologic systems. Cyclosporine inhibits production of interleukin-2, the cytokine responsible for inducing T-Cell proliferation. Psoriasis skin lesions can recur within days to weeks after this systemic treatment is stopped. Adverse effects include hypertension, impaired renal function, and an increased risk of cancer. Acitretin is a second generation oral retinoid. The use of oral retinoid therapy has shown limited efficacy for chronic stable plaque psoriasis.

Thus, while there are a number of treatments for psoriasis currently available, they all are accompanied by various side effects, high costs, and long complicated treatment protocols. Accordingly, there remains a need in the art for more effective and otherwise improved methods for treating dermatological conditions related to psoriasis. The present invention fulfills this need and provides further related advantages.

Previously Identified Genes and Loci

Familial aggregation, twin studies and consistent ethnic differences in disease frequency have strongly supported the important role of genetic factors in the cause of psoriasis Studies of twins have shown the heritability of psoriasis to be 70-90%. Such familial investigations clearly indicate that there are strong genetic factors associated with psoriatic pathogenesis. Several psoriasis-susceptibility loci have been reported on chromosomes: 6p21.3 (PSORS1) (Trembath et al., 1997; Zhang et al., 2002); 17q25 (PSORS2) (Tomfohrde et al., 1994), 4q34 (PSORS3) (Matthews et al. 1996), 1q (PSORS4) (Capon et al., 1999), and 3q (PSORS5) (Samuelsson et al., 1999), 19p13 (PSORS6) (Lee et al., 2000), 1p (PSORS7) (Vael et al., 2001), 4q31 (PSORS9) (Zhang et al., 2002), 4q13 (Samuelsson et al., 1999), 16q, 20p (Nair, 1997), 8q (Trembath et al., 1997), 2p (Veal et al., 2001), 2q (Trembath et al., 1997), 7 (Veal et al., 2001), 9q33 (Zhang et al., 2002), 14q (Veal et al., 2001), 15 (Samuelsson et al., 1999) and 18p11 (Asumalahti et al., 2003).

These 19 loci are located on 15 different chromosomes and of these regions, only one locus, 6p21, has been consistently implicated in psoriasis (reviewed in Sagoo et al., 2004). Thus, there is a continuing need in the medical arts for genetic markers of psoriasis and guidance for the use of such markers.

Genome Wide Association Study to Construct a GeneMap for Psoriasis Disease

The present invention is based on the discovery of genes associated with psoriasis. In the preferred embodiment, disease-associated loci (candidate regions; Table 1) are therefore identified by the statistically significant differences in haplotype frequencies between the cases and the controls. For the purpose of the present invention, novel candidate regions showing a difference with a −log 10 P value of 3.0 or higher are identified along with previously known regions that have been previously reported to be associated with psoriasis.

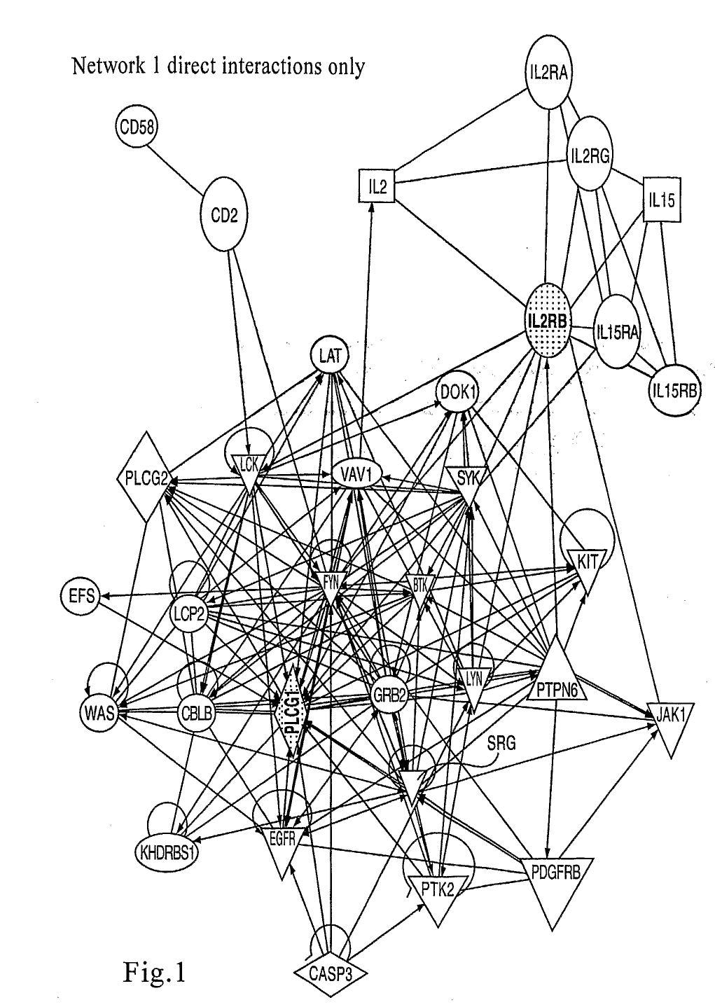

The invention provides a method for the discovery of genes associated with psoriasis and the construction of a GeneMap (see FIGS. 1-13 herein) for psoriasis disease in a human population, comprising the following steps (see also Example section herein):

Step 1: Recruit Patients (Cases) and Controls

In the preferred embodiment, 500 patients diagnosed for psoriasis along with two family members are recruited from the Quebec Founder Population (QFP). The preferred trios recruited are parent-parent-child (PPC) trios. Trios can also be recruited as parent-child-child (PCC) trios.

In another embodiment, the present invention is performed as a whole or partially with DNA samples from individuals of another founder population than the Quebec population or from the general population.

Step 2: DNA Extraction and Quantitation

Any sample comprising cells or nucleic acids from patients or controls may be used. Preferred samples are those easily obtained from the patient or control. Such samples include, but are not limited to blood, peripheral lymphocytes, buccal swabs, epithelial cell swabs, nails, hair, bronchoalveolar lavage fluid, sputum, or other body fluid or tissue obtained from an individual.

In the preferred embodiment, DNA is extracted from such samples in the quantity and quality necessary to perform the invention using conventional DNA extraction and quantitation techniques. The present invention is not linked to any DNA extraction or quantitation platform in particular.

Step 3: Genotype the Recruited Individuals

In the preferred embodiment, assay specific and/or locus-specific and/or allele-specific oligonucleotides for every SNP marker of the present invention (Tables 2-10 and 13) are organized onto one or more arrays. The genotype at each SNP locus is revealed by hybridizing short PCR fragments comprising each SNP locus onto these arrays. The arrays permit a high-throughput genome wide association study using DNA samples from individuals of the Quebec founder population. Such assay-specific and/or locus-specific and/or allele-specific oligonucleotides necessary for scoring each SNP of the present invention are preferably organized onto a solid support. Such supports can be arrayed on wafers, glass slides, beads or any other type of solid support.

In another embodiment, the assay-specific and/or locus-specific and/or allele-specific oligonucleotides are not organized onto a solid support but are still used as a whole, in panels or one by one. The present invention is therefore not linked to any genotyping platform in particular.

In another embodiment, one or more portions of the SNP maps (publicly available maps, proprietary maps from Perlegen Sciences, Inc. (Mountain View, Calif., USA), and our own proprietary QLDM map) are used to screen the whole genome, a subset of chromosomes, a chromosome, a subset of genomic regions or a single genomic region.

The 1,500 individuals composing the 500 trios are preferably individually genotyped with at least 80,000 markers, generating at least a few million genotypes; more preferable, at least a hundred million.

Step 4: Exclude the Markers that did not Pass the Quality Control of the Assay.

Preferably, the quality controls consist of, but are not limited to, the following criteria: SNPs that had a high rate of Mendelian errors (cut-off at 1% Mendelian error rate), that deviate from the Hardy-Weinberg equilibrium, that have too many missing data (cut-off at 1% missing values or higher), or simply because they are non-polymorphic in the Quebec founder population (cut-off at 10% MAF, or below).

Step 5: Perform the Genetic Analysis on the Results Obtained using Haplotype Information as Well as Single-Marker Association.

In the preferred embodiment, genetic analysis is performed on all the genotypes from step 3.

In another embodiment, genetic analysis is performed on a total of 80,654 SNPs.

In one embodiment, the genetic analysis consists of, but is not limited to the features corresponding to Phase information and haplotype structures. Phase information and haplotype structures are preferably deduced from trio genotypes using Phasefinder. Since chromosomal assignment (phase) can not be estimated when all trio members are heterozygous, an Expectation-Maximization (EM) algorithm may be used to resolve chromosomal assignment ambiguities after Phasefinder.

In yet another embodiment, the PL-EM algorithm (Partition-Ligation EM; Niu et al., Am. J. Hum. Genet. 70:157 (2002)) can be used to estimate haplotypes from the “genotype” data as a measured estimate of the reference allele frequency of a in 15-marker windows that advance in increments of one marker across the data set. The results from such algorithms are converted into 15-marker haplotype files. Subsequently, the individual 15-marker block files are assembled into one continuous block of haplotypes for the entire chromosome. These extended haplotypes can then be used for further analysis. Such haplotype assembly algorithms take the consensus estimate of the allele call at each marker over all separate estimations (most markers are estimated 15 different times as the 15 marker blocks pass over their position).

In the preferred embodiment, the haplotypes for both the controls and the patients are derived in this manner. The preferred control of a trio structure is the spouse if the patient is one of the parents or the non-transmitted chromosomes (chromosomes found in parents but not in affected child) if the patient is the child.

In another embodiment, the haplotype frequencies among patients are compared to those among the controls using LDSTATS, a program that assesses the association of haplotypes with the disease. Such program defines haplotypes using multi-marker windows that advance across the marker map in one-marker increments. Such windows can be 1, 3, 5, 7 or 9 markers wide, and all these window sizes are tested concurrently. At each position the frequency of haplotypes in cases is compared to the frequency of haplotypes in controls. Such allele frequency differences for single marker windows can be tested using Pearson's Chi-square with one degree of freedom. Multi-allelic haplotype association can be tested using Smith's normalization of the square root of Pearson's Chi-square. Such significance of association can be reported in two ways:

The significance of association within any one haplotype window is plotted against the marker that is central to that window.

P-values of association for each specific marker are calculated as a pooled P-value across all haplotype windows in which they occur. The pooled P-value is calculated using an expected value and variance calculated using a permutation test that considers covariance between individual windows. Such pooled P-values can yield narrower regions of gene location than the window data (see example 3 for details on analysis methods, such as LDSTATs V2.0 and V4.0).

In another embodiment, conditional haplotype analyses can be performed on subsets of the original set of cases and controls using the program LDSTAT. The selection of a subset of cases and their matched controls can be based on the carrier status of cases at a gene or locus of interest (see conditional analysis section in example 3 herein). Various conditional haplotypes can be derived, such as protective haplotypes and risk haplotypes.

Step 6: Fine Mapping

In this step, the candidate regions that were identified by step 4 are further mapped for the purpose of refinement and validation.

In the preferred embodiment, this fine mapping is performed with a density of genetic markers higher than in the genome wide scan (step 3) using any genotyping platform available in the art. Such fine mapping can be but is not limited to typing the allele via an allele-specific elongation assay that is then ligated to a locus-specific oligonucleotide. Such assays can be performed directly on the genomic DNA at a highly multiplex level and the products can be amplified using universal oligonucleotides. For each candidate region, the density of genetic markers, can be but is not limited to a set of SNP markers with an average inter-marker distance of 1 to 4 Kb distributed over about 400 Kb to 1 Mb roughly centered at the highest point of the GWS curves was selected. The preferred samples are those obtained from psoriasis disease PPC trios including the ones used for the GWS. Other preferred samples are trios or case control samples from another population

In the preferred embodiment, the genetic analysis of the results obtained using haplotype information as well as single-marker association (as performed as in step 3, described herein). The candidate regions validated and confirmed after this analysis are processed to a gene mining step described in example 5 to characterize it's marker and genetic content.

Step 7: SNP and DNA Polymorphism Discovery

In the preferred embodiment, all the candidate genes and regions identified in step 6 are sequenced for polymorphism identification.

In another embodiment, the entire region, including all introns, is sequenced to identify all polymorphisms.

In yet another embodiment, the candidate genes are prioritized for sequencing, and only functional gene elements (promoters, conserved non-coding exons and splice sites) are sequenced.

In yet another embodiment, previously identified polymorphisms in the candidate regions can also be used. For example, SNPs from dbSNP, Perlegen Sciences, Inc., or others can also be used rather than resequencing the candidate regions to identify polymorphisms.

The discovery of SNPs and DNA polymorphisms generally comprises a step consisting of determining the major haplotypes in the region to be sequenced. The preferred samples are selected according to which haplotypes contribute to the association signal observed in the region to be sequenced. The purpose is to select a set of samples that covers all the major haplotypes in the given region. Each major haplotype is preferably present in at least a few copies.

Any analytical procedure may be used to detect the presence or absence of variant nucleotides at one or more polymorphic positions of the invention. In general, the detection of allelic variation requires a mutation discrimination technique, optionally an amplification reaction and optionally a signal generation system. Any means of a mutation detection or discrimination may be used. For instance, DNA sequencing, scanning methods, hybridization, extension based methods, incorporation based methods, restriction enzyme-based methods and ligation-based methods may be used in the methods of the invention.

Sequencing methods include, but are not limited to, direct sequencing, and sequencing by hybridization. Scanning methods include, but are not limited to, protein truncation test (PTT), single-strand conformation polymorphism analysis (SSCP), denaturing gradient gel electrophoresis (DGGE), temperature gradient gel electrophoresis (TGGE), cleavage, heteroduplex analysis, chemical mismatch cleavage (CMC), and enzymatic mismatch cleavage. Hybridization-based methods of detection include, but are not limited to, solid phase hybridization such as dot blots, multiple allele specific diagnostic assay (MASDA), reverse dot blots, and oligonucleotides arrays (DNA Chips). Solution phase hybridization amplification methods may also be used, such as Taqman. Extension based methods include, but are not limited to, amplification refraction mutation systems (ARMS), amplification refractory mutation systems (ALEX), and competitive oligonucleotides priming systems (COPS). Incorporation based methods include, but are not limited to, mini-sequencing and arrayed primer extension (APEX). Restriction enzyme-based detection systems include, but are not limited to restriction site generating PCR. Lastly, ligation based detection methods include, but are not limited to, oligonucleotides ligation assay (OLA). Signal generation or detection systems that may be used in the methods of the invention include, but are not limited to, fluorescence methods such as fluorescence resonance energy transfer (FRET), fluorescence quenching, fluorescence polarization as well as other chemiluminescence, electrochemiluminescence, Raman, radioactivity, colometric methods, hybridization protection assays and mass spectrometry methods. Further amplification methods include, but are not limited to self sustained replication (SSR), nucleic acid sequence based amplification (NASBA), ligase chain reaction (LCR), strand displacement amplification (SDA) and branched DNA (B-DNA).

Step 8: Ultrafine Mapping

This step further maps the candidate regions and genes confirmed in the previous step to identify and validate the responsible polymorphisms associated with psoriasis in the human population.

In a preferred embodiment, the discovered SNPs and polymorphisms of step 7 are ultrafine mapped at a higher density of markers than the fine mapping described herein using the same technology described in step 6.

Step 9: GeneMap Construction

The confirmed variations in DNA (including both genic and non-genic regions) are used to build a GeneMap for psoriasis disease. The gene content of this GeneMap is described in more detail below. Such GeneMap can be used for other methods of the invention comprising the diagnostic methods described herein, the susceptibility to psoriasis, the response to a particular drug, the efficacy of a particular drug, the screening methods described herein and the treatment methods described herein.

As is evident to one of ordinary skill in the art, all of the above steps or the steps do not need to be performed, or performed in a given order to practice or use the SNPs, genomic regions, genes, proteins, etc. in the methods of the invention.

Genes from the GeneMap

In the preferred embodiment the GeneMap consists of genes and targets, in a variety of combinations, identified from the candidate regions listed in Table 1. Such genes's are briefly described in Tables 10-12 and 14. In the preferred embodiment, all genes from Tables 10-12 and 14 are present in the GeneMap.

The genes of the invention are arranged by candidate regions and by their chromosomal location. Such order is for the purpose of clarity and does not reflect any other criteria of selection in the association of the genes with psoriasis.

The genes of the invention were also evaluated using the Ingenuity Pathway Analysis application (IPA, Ingenuity systems) in order to identify direct biological interactions between these genes, and also to identify molecular regulators acting on those genes (indirect interactions) that could also be involved in psoriasis. The purpose of this effort was to decipher the molecules involved in contributing to psoriasis susceptibility. These gene interaction networks are very valuable tools in the sense that they facilitate extension of the map of gene products that could represent potential drug targets for psoriasis.

A selection of 15 genes from Tables 10-12 was performed based on the results of the genetic analyses; those genes were considered for the development of potential protein interaction networks involved in psoriasis. These genes were imported in the IPA software and are presented in Table A below.

TABLE A
Input list of genes

| Region ID | Entrez Gene ID | Gene Symbol |
|---|---|---|
| 78, 491, 492 | 8829 | NRP1 |
| 131 | 3560 | IL2RB |
| 99, 570, 571, 572, 573 | 949 | SCARB1 |
| 110, 619 | 2567 | GABRG3 |
| 32, 278, 279, 280, 281, 282, | 56034 | PDGFC |
| 283, 284, 285 | | |
| 73, 480, 481, 482 | 1539 | CYLC2 |
| 47, 343, 344, 345, 346, 300 | 51000 | SLC35B3 |
| 27, 255, 256 | 170712 | COX7B2 |
| 107, 613 | 79890 | RIN3 |
| 100, 582, 583, 584, 585 | 390386 | LOC390386 |
| 100, 582, 583, 584, 585 | 56163 | RNF17 |
| 42, 314, 315 | 353189 | SLCO4C1 |
| 42, 314, 315, 316 | 133482 | SLCO6A1 |
| 40, 306, 307, 308, 309, 310, | 2745 | GLRX |
| 311 | | |
| 125, 687, 688, 689, 690, 000 | 5335 | PLCG1 |

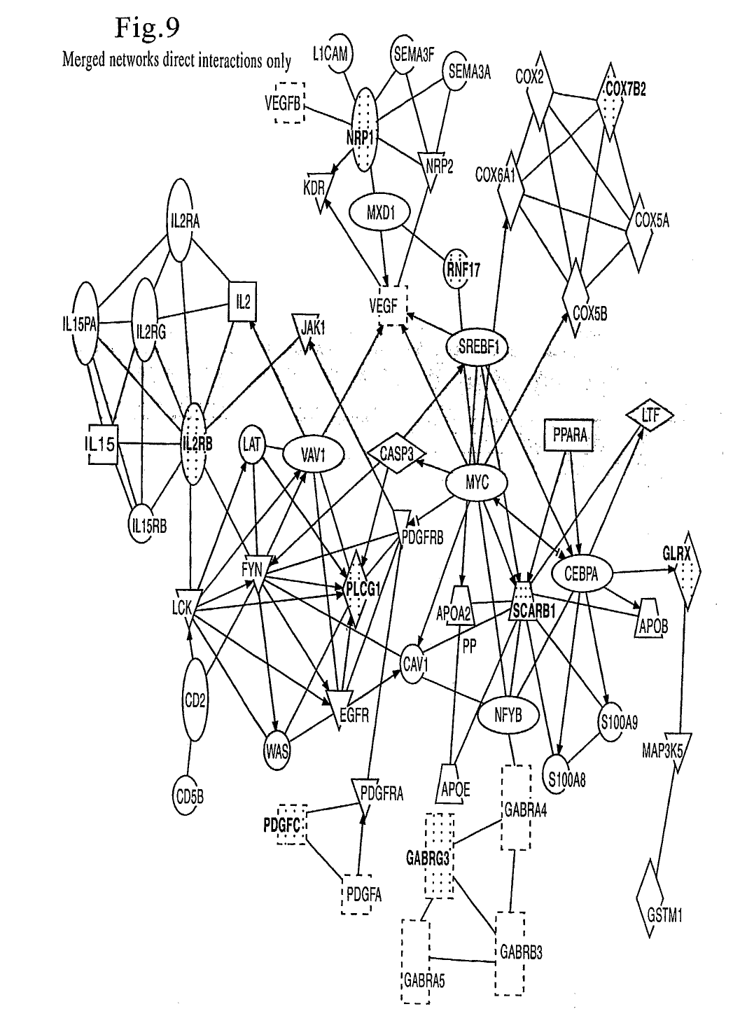

In a first step, the analysis was performed by looking for direct interactions only. From this analysis 97 genes were mapped to the Ingenuity database and assigned to 8 networks as defined by IPA (primary networks are displayed in FIGS. 1-8). These networks are based on functional relationships between gene products using known interactions in the literature. For each individual network, some nodes were manually extended to include good candidate genes that could play a role in the biochemical pathways of psoriasis. Table B below contains information about the gene content of each network, as well as the top functions assigned to those biochemical pathways. The fusion of those 8 networks resulted in a unique network which likely includes some of the critical pathways involved in the pathogenesis of psoriasis. After having been cleaned for sake of clarity, this network is presented in FIG. 9.

TABLE B
Genetic networks associated with genes identified by the WGAS on psoriasis.
Direct interactions only. Bold genes are those identified by the WGAS

| | | | Genes manually added |
|---|---|---|---|
| Networks | Genes in Ingenuity network* | Top functions | to the networks |
| 1 | BTK, CASP3, CBLB, DOK1, EFS, | Cellular growth and | CD2, CD58, IL2, |
| | EGFR, FYN, GRB2, IL2RB, | proliferation, cell siganling, | IL2RA, IL2RG, IL15, |
| | KHDRBS1, KIT, LAT, LCK, LCP2, | molecular transport | IL15RA, IL15RB, |
| | LYN, PDGFRB, PLCG1, PLCG2, | | JAK1 |
| | PTK2, PTPN6, SRC, SYK, VAV1, | | |
| | WAS | | |
| 2 | MXD1, RNF17 | Cancer, cell morphology, | N/A |
| | | connective tissue disorders | |
| 3 | CEBPA, GLRX, MAP3K5 | Cell cycle, connective tissue | GSTM1 |
| | | development and function, | |
| | | drug metabolism | |
| 4 | PDGFA, PDGFC, PDGFRA | Digestive system | N/A |
| | | development and function, | |
| | | embryonic development, | |
| | | tissue morphology | |
| 5 | GABRA1, GABRA2, GABRA3, | Cell signaling, molecular | N/A |
| | GABRA4, GABRA5, GABRA6, | transport, nervous system | |
| | GABRB1, GABRB2, GABRB3, | development and function | |
| | GABRG2, GABRG3 | | |
| 6 | HHEX, KDR, L1CAM, NRP1, NRP2, | Cell morphology, cellular | N/A |
| | PLXNA1, RGS19IP1, SEMA3A, | compromise, neurological | |
| | SEMA3C, SEMA3D, SEMA3E, | disease | |
| | SEMA3F, VEGF, VEGFB | | |
| 7 | COX1, COX2, COX3, COX4I1, | Neurological disease, genetic | N/A |
| | COX4I2, COX5A, COX5B, COX6A1, | disorder, metabolic disease | |
| | COX6A2, COX6B1, COX6B2, | | |
| | COX6C, COX7A1, COX7A2, | | |
| | COX7A2L, COX7B, COX7B2, | | |
| | COX7C, COX8A, COX8C | | |
| 8 | APOA1, APOA2, APOB, APOC2, | Lipid metabolism, molecular | N/A |
| | APOE, CAV1, HDL3, LTF, MYC, | transport, small molecule | |
| | NFYB, NR0B1, NR5A1, NR5A2, | biochemistry | |
| | PDZK1, PPARA, S100A8, S100A9, | | |
| | SAA1, SCARB1, SREBF1 | | |

Network 1 contains 33 nodes (24 original and 9 manual additions) and includes 2 genes from the fine mapped regions (FIG. 1). A short description of these 2 genes follows. By virtue of their role in immune response and/or inflammation, several genes from this network are very good candidates for involvement in the pathophysiology of psoriasis. One gene in this network, LYN, was shown to be upregulated in psoriatic skin compared to control skin in a gene expression profiling study (Zhou et al 2003).

IL2RB

IL2RB is part of the IL-2 receptor, which is produced by activated T cells. The interleukin 2 receptor, which is involved in T cell-mediated immune responses, is present in 3 forms with respect to its ability to bind interleukin 2. The low affinity form is a monomer of the alpha subunit and is not involved in signal transduction. The intermediate affinity form consists of an alpha/beta subunit heterodimer, while the high affinity form consists of an alpha/beta/gamma subunit heterotrimer. Both the intermediate and high affinity forms of the receptor are involved in receptor-mediated endocytosis and transduction of mitogenic signals from interleukin 2. The protein encoded by the IL2RB gene represents the beta subunit and is a type I membrane protein. Cytokines IL-2 (made by activated T cells) and IL-12 (made by mature Langerhans cells) bind to T-cells; this event regulates mitotic activation and differentiation of T cells into type 1 effectors. IL-2 binds to T-cells which promotes the differentiation of T cells into type 1 effectors (Th1). Also, the IL2R beta and IL2R gamma chains are shared by receptors of IL15 and IL2. This forms the basis of many overlapping biological activities of IL15 and IL2. The IL2 receptor requires an additional IL2-specific alpha subunit for high affinity IL2 binding. The IL15R alpha chain is structurally related to IL2R alpha, but is capable of binding IL15 with high affinity, independent of other subunits, which suggests distinct roles for IL15 and IL2.

PLCG1:

The protein encoded by the PLCG1 gene catalyzes the formation of inositol 1,4,5-trisphosphate and diacylglycerol from phosphatidylinositol 4,5-bisphosphate. This reaction uses calcium as a cofactor and plays an important role in the intracellular transduction of receptor-mediated tyrosine kinase activators. For example, when activated by SRC, the encoded protein causes the Ras guanine nucleotide exchange factor RasGRP1 to translocate to the Golgi, where it activates Ras. Also, this protein has been shown to be a major substrate for heparin-binding growth factor 1 (acidic fibroblast growth factor)-activated tyrosine kinase. Bivona et al (2003) demonstrated that, in response to Src-dependent activation of PLCG1, the Ras guanine nucleotide exchange factor RasGRP1 translocates to the Golgi, where it activates Ras. Whereas calcium positively regulated Ras on the Golgi apparatus through RasGRP1, the same second messenger negatively regulated Ras on the plasma membrane by means of the Ras GTPase-activating protein CAPRI. Ras activation after T-cell receptor stimulation in Jurkat cells, rich in RasGRP1, was limited to the Golgi apparatus through the action of CAPRI, unambiguously demonstrating a physiological role for Ras in Golgi apparatus function. Activation of Ras in the Golgi also induced differentiation of PC12 cells, transformed fibroblasts, and mediated radioresistance. Thus, Bivona et al (2003) concluded that activation of Ras in the Golgi apparatus has important biologic consequences and proceeds through a pathway distinct from the one that activates Ras on the plasma membrane. Interestingly, PLCG1 plays an important function in keratinocytes: it has been shown that intracellular PLCG1-mediated Ca2+ mobilization plays a critical role in regulating keratinocyte differentiation (Bourguignon et al 2004). Also, it has been reported that cells lacking PLCG1 fail to activate NF-kB in response to T cell co-stimulation (Dienz et al 2003). Finally, PLCG1 plays a critical role in the signaling pathway leading to Rap1 activation triggered by the TCR (Katagiri et al 2004). Lymphocyte-expressed Rap1 is a key modulator of T cell activation and trafficking.

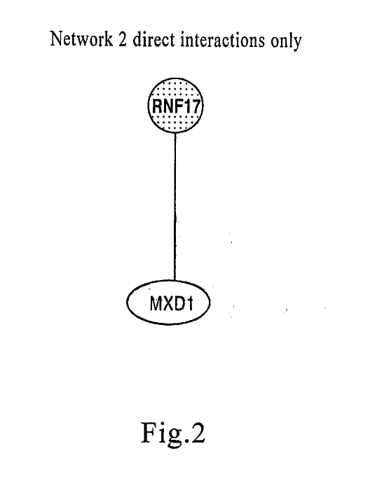

Network 2 contains 2 original nodes and includes 1 gene from the fine mapped regions (FIG. 2). A short description of this gene follows. In this network, MXD1 was shown to be upregulated in psoriatic skin compared to control skin in a gene expression profiling study (Zhou et al 2003).

RNF17

This gene is also known under the name Mmip-2 and encodes a ring finger protein that interacts with mad proteins. Members of the mad family are basic-helix-loop-helix-leucine zipper proteins which inhibit the transcriptional activity of c-Myc. The inhibition of mad proteins by Mmip-2/Rnf-17 modulates c-Myc function by increasing its ability to regulate a subset of its potential target genes (Yin et al 2001). Interestingly, after skin injury the expression of mad1 mRNA and protein, but not of other mad genes, increases (Werner et al 2001). Thus, by regulating mad protein function, and hence myc function, RNF17 is a very good candidate gene to play a role in the pathogenesis of psoriasis.

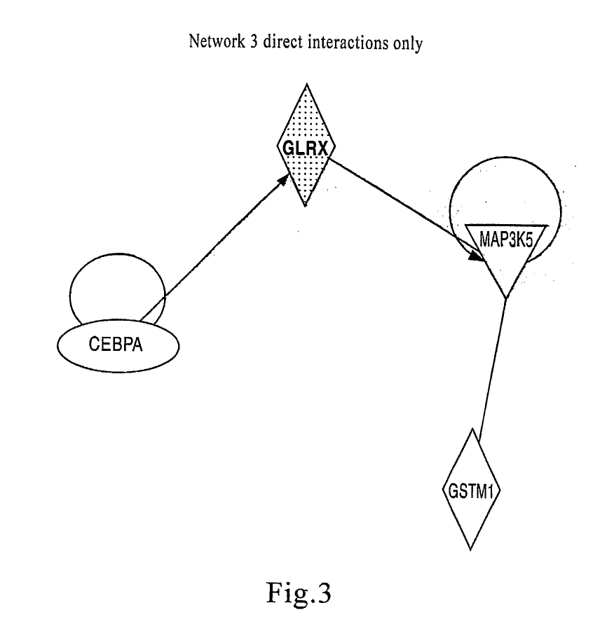

Network 3 contains 4 nodes (3 original and 1 manual addition) and includes the GLRX gene from the fine mapped regions (FIG. 3).

GLRX

This gene encodes glutaredoxin which is involved in protection against oxidative stress. It has been shown that dendritic cell presentation of antigens to T-cells causes an elevation in intracellular oxidation states in both cells. If this elevated oxidation state is inhibited artificially by modulating the thioredoxin and glutaredoxin pathways, then DC-induced proliferation and cytokine production by T cells as well as T cell-induced cytokine production by DCs is inhibited. Glutaredoxin has a prominent role in homeostasis of protein sulfhydryl groups, both in a protective mode under overt oxidative stress associated with aging and various disease states (including cardiovascular and neurodegenerative diseases, diabetes, AIDS, and cancer). It also has a regulatory mode whereby reversible glutathionylation represents a mechanism of redox-activated signal transduction. GLRX is also involved in the suppression of apoptosis through ASK1 (apoptosis signal-regulating kinase 1) (Raghavachari et al., 2001). ASK1 is also known as MAP3K5 (refer to FIG. 3), and has been shown to be an intracellular regulator of keratinocyte differentiation (Sayama et al 2001).

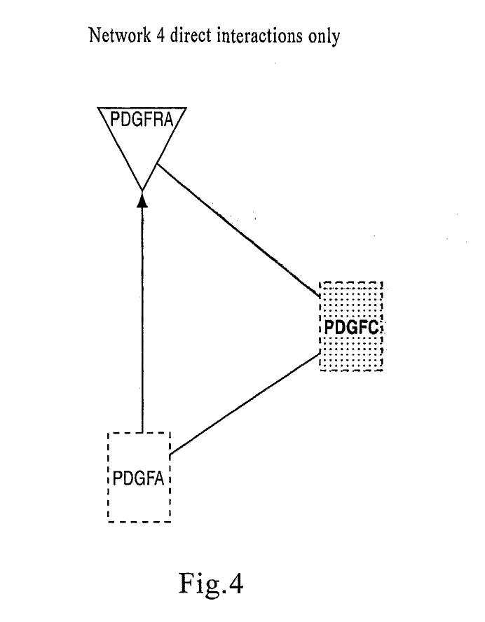

Network 4 contains 3 original nodes and includes the PDGFC gene from the fine mapped regions (FIG. 4).

PDGFC

The protein encoded by the PDGFC gene is a member of the platelet-derived growth factor family. It differs from the platelet-derived growth factor alpha and beta polypeptides in having an unusual N-terminal domain, the CUB domain. Indeed, in contrast to PDGF-A and -B, which are secreted as bioactive dimers after intracellular processing, the PDGFC precursor polypeptide is secreted intact from the cell and requires extracellular proteolytic cleavage of the receptor interacting domain (the CUB domain) to produce the active growth factor. The multidomain serine protease tissue plasminogen activator (tPA) has been shown to cleave and activate PDGFC precursors (Fredricksson et al 2005). In its C-terminal region, PDGFC contains the growth factor domain (GFD) which shows some homology to VEGF and PDGF-A chain. The GFD has a potent biological activity. PDGFC-expressing transgenic mice had enlarged livers associated with increased fibrosis, steatosis, cell dysplasia, and hepatocellular carcinomas. These studies indicate that hepatic expression of PDGFC induces a number of profibrotic pathways, suggesting that this growth factor may act as an initiator of fibrosis. Platelet-derived growth factor (PDGF) is a potent mitogenic and chemotactic factor for fibroblasts and other cell types. PDGF effects are mediated by binding of PDGF to dimeric PDGF receptors possessing intrinsic tyrosine kinase activity. The expression pattern of PDGF receptors was recently analyzed in cryostat sections of normal and growth-activated human skin. PDGF receptors are expressed at low levels in normal skin. In contrast, PDGF receptor expression is greatly elevated in the dermis of growth-activated skin from chronic wounds and psoriatic lesions, and is also increased in dermal fibroblasts and in dermal blood vessels in both conditions. Differential expression of PDGF receptors could regulate increased proliferation of vascular and connective tissue cells observed in psoriasis and chronic wounds (Krane et al., 1991). Platelet-derived growth factors, such as PDGFC, are major mitogens and chemotactic factors for fibroblasts and other cell types (Jinnin et al., 2005). Members of the PDGF ligand family are known to play important roles in wound healing and fibrotic disease. PDGFC is activated by proteolysis and induces proliferation of fibroblasts when overexpressed in transgenic mice (Campbell et al., 2005).

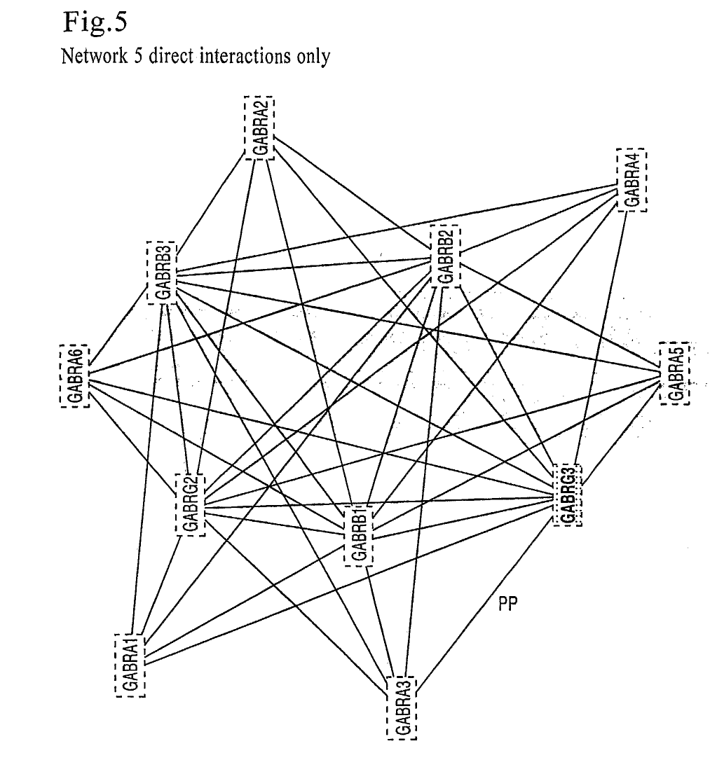

Network 5 contains 11 original nodes and includes the GABRG3 gene from the fine mapped regions (FIG. 5).

GABRG3

The gamma-aminobutyric acid (GABA) A receptor, gamma 3 is related to an ion channel. GABA is the major inhibitory neurotransmitter of the brain and acts through binding to GABA A receptors, where the ligand causes an influx of chloride ions.

One study reported the presence of a GABA A-like receptor in keratinocytes where it was shown to play an important role in barrier homeostasis. Activation of this receptor by GABA improved epidermal hyperplasia when skin was traumatized and thus accelerated skin barrier recovery (Denda et al 2002).

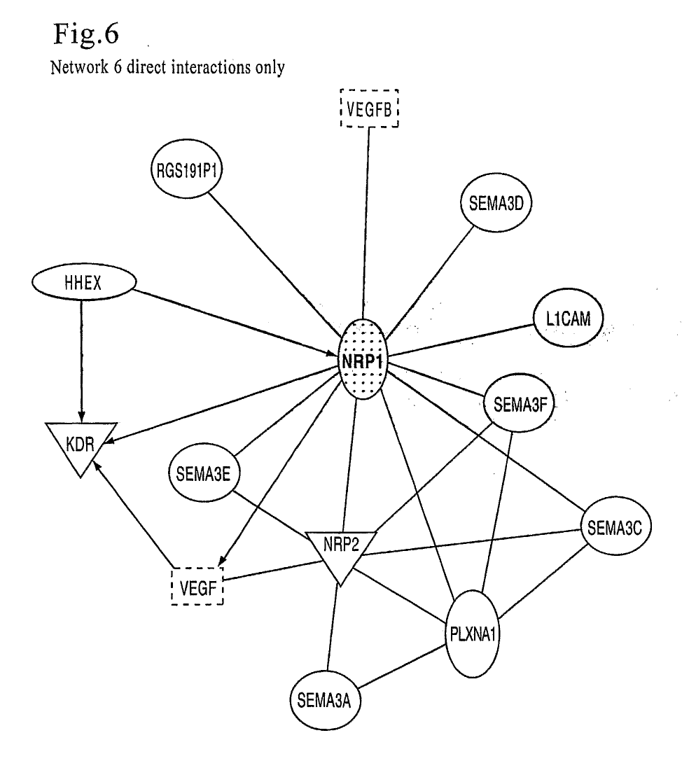

Network 6 contains 14 original nodes and includes the NRP1 gene from the fine mapped regions (FIG. 6).

NRP1

The neuropilin 1 gene encodes a membrane-bound coreceptor to a tyrosine kinase receptor for both vascular endothelial growth factor (VEGF), an angiogenesis factor, and semaphorin 3A (SEMA3A), a mediator of axonal guidance. NRP1 can play a role in angiogenesis, axon guidance, cell survival, migration, and invasion. NRP1 has been shown to be expressed in keratinocytes in vitro and in vivo. In HaCaT cells, a keratinocyte cell line, transcriptional repression of the NRP1 gene by the neuron restrictive silencer factor NRSF reduced the Sema3A mediated inhibition of HaCaT keratinocyte migration (Kurschat et al 2006). In addition, NRP1 has been involved in interactions between dendritic cells (DCs) and T cells that are essential for initiation of the primary immune response. Preincubation of DCs or T cells with blocking NRP1 antibodies inhibits DC-induced proliferation of resting T cells (Tordjman et al 2002).

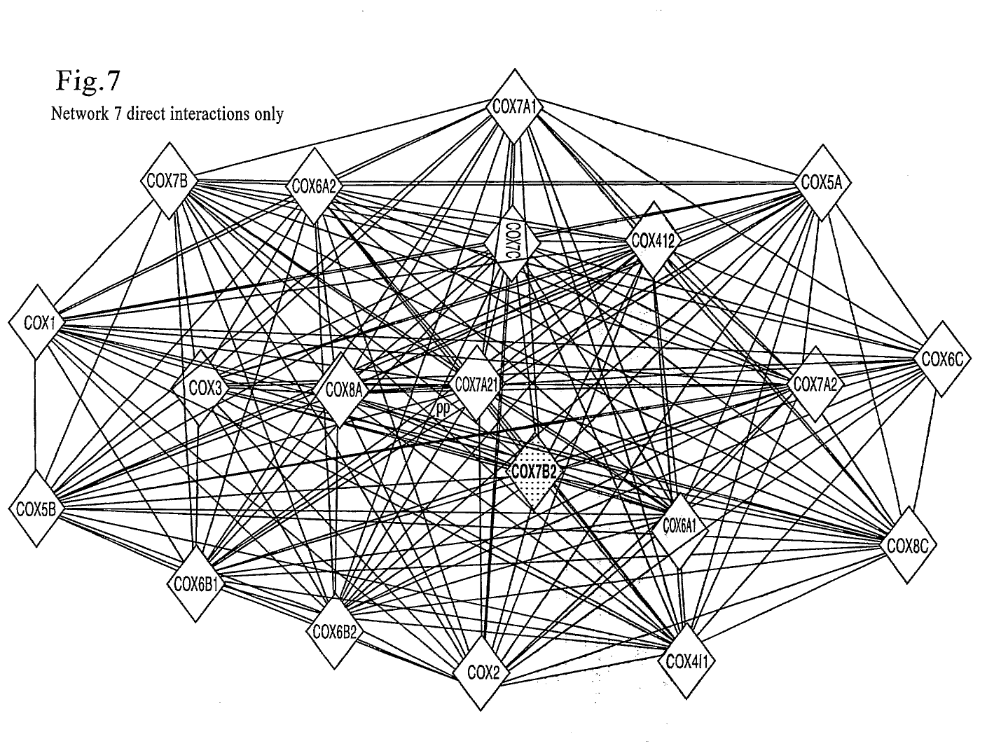

Network 7 contains 20 original nodes and includes the COX7B2 gene from the fine mapped regions (FIG. 7).

COX7B2

This gene encodes the cytochrome c oxidase subunit VIIb2, the terminal oxidase in mitochondrial electron transport, and is involved in oxidative phosphorylation.

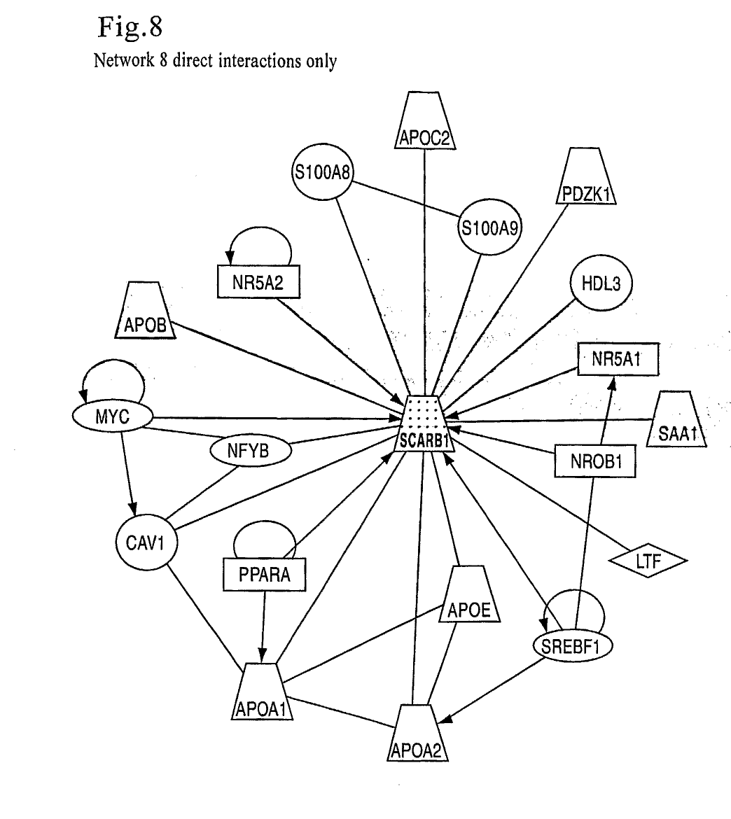

Network 8 contains 20 original nodes and includes the SCARB1 gene from the fine mapped regions (FIG. 8). The expression of 6 genes from this network has been shown to vary in one study of gene expression profiling for psoriasis. In psoriatic versus control skin, S100A8, S100A9, LTF, and MYC were upregulated, and PDZK1 and APOE were downregulated (Zhou et al 2003).

SCARB1

This gene encodes the scavenger receptor class B, member 1 protein. It is a cell surface receptor for high density lipoproteins (HDL) and mediates the selective uptake of cholesterol from circulating HDL. SCARB1 has been shown to be expressed in cultured human keratinocytes and epidermis and its expression is regulated in response to changes in cholesterol homeostasis and barrier requirements (Tsuruoka et al 2002). The epidermis is an active site for cholesterol synthesis which, with ceramides and fatty acids, helps the skin to maintain its function as a permeability barrier.

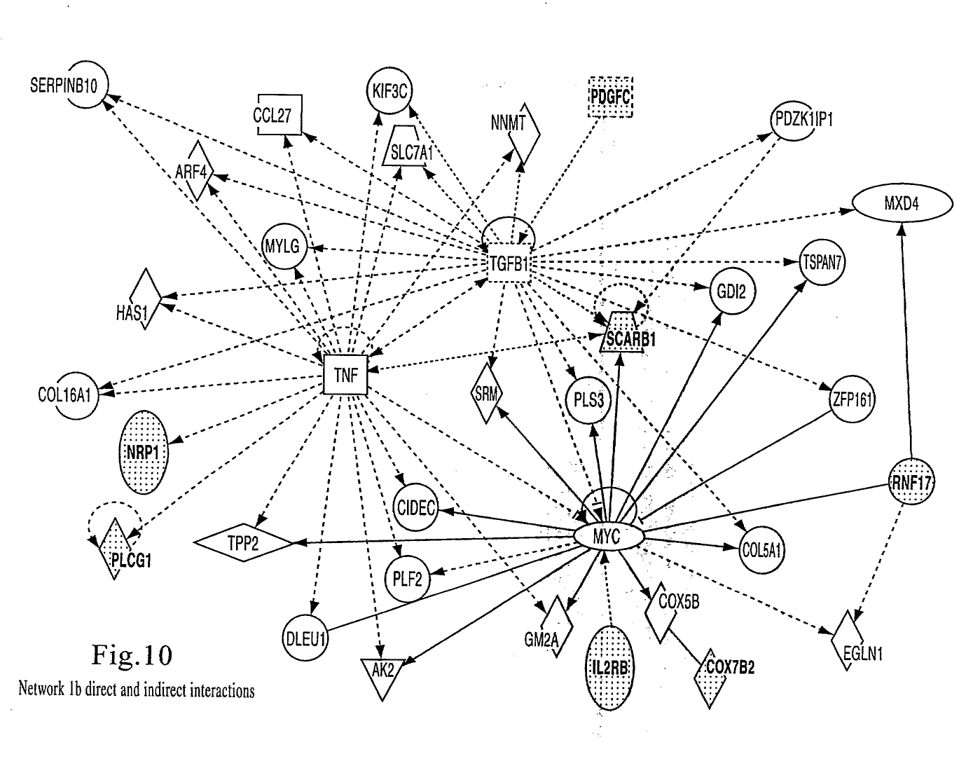

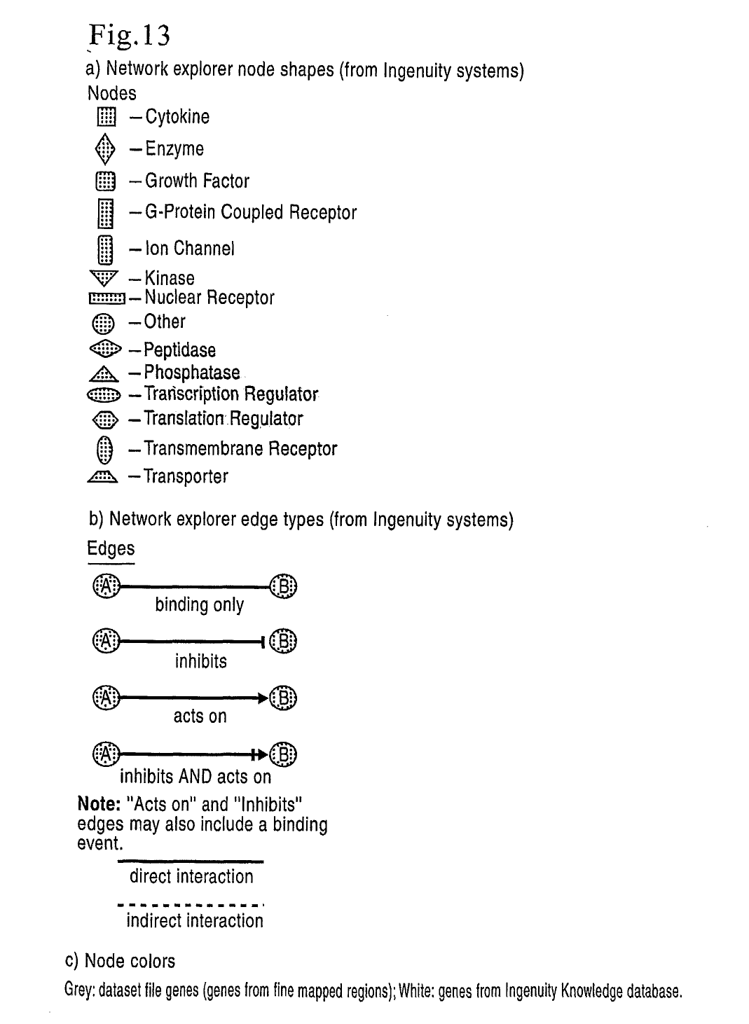

In a second step, the analysis was performed by looking for direct and indirect interactions. From this analysis 55 genes were mapped to the Ingenuity database and assigned to 3 genetic networks as defined by IPA (primary networks are shown in FIGS. 10-12). Table C contains information about the gene content of each network, as well as the top functions assigned to those biochemical pathways. FIG. 13 provides a description of the symbols used in the Ingenuity networks.

TABLE C
Genetic networks associated with genes identified by the WGAS on psoriasis. Direct
and indirect interactions. Bold genes are those identified by the WGAS.

| Networks | Genes in Ingenuity network* | Top functions |
|---|---|---|
| 1b | AK2, ARF4, CCL27, CIDEC, COL5A1, | Cell death, hepatic system disease, |
| | COL16A1, COX5B, COX7B2, DLEU1, | connective tissue development and |
| | EGLN1, GDI2, GM2A, HAS1, IL2RB, | function |
| | KIF3C, MXD4, MYC, MYL6, NNMT, NRP1, | |
| | PDGFC, PDZK1IP1, PLCG1, PLF2, PLS3, | |
| | RNF17, SCARB1, SERPINB10, SLC7A1, | |
| | SRM, TGFB1, TNF, TPP2, TSPAN7, ZFP161 | |
| 2b | CAT, CEBPA, GLRX, HOXA9, MAP3K5, | Cell death, cellular development, |
| | MAPK8, PTPN1, TH, TNFSF11 | hematological system development |
| | | and function |
| 3b | GABRA1, GABRA2, GABRA3, GABRA4, | Neurological disease, psychological |
| | GABRA5, GABRA6, GABRB1, GABRB2, | disorders, cell signaling |
| | GABRB3, GABRG2, GABRG3 | |

Network 1b contains 35 original nodes and includes 7 genes from the fine mapped regions (FIG. 10). The expression of 6 genes from this network has been shown to vary in one study of gene expression profiling for psoriasis. In psoriatic versus control skin, NNMT, PDZK1IP1, PLS3, MYC, EGLN1, and GM2A were upregulated (Zhou et al 2003). For descriptions of the COX7B2, IL2RB, NRP1, PDGFC, PLCG1, RNF17, and SCARB1 genes, please refer to text above about networks from direct analysis only.

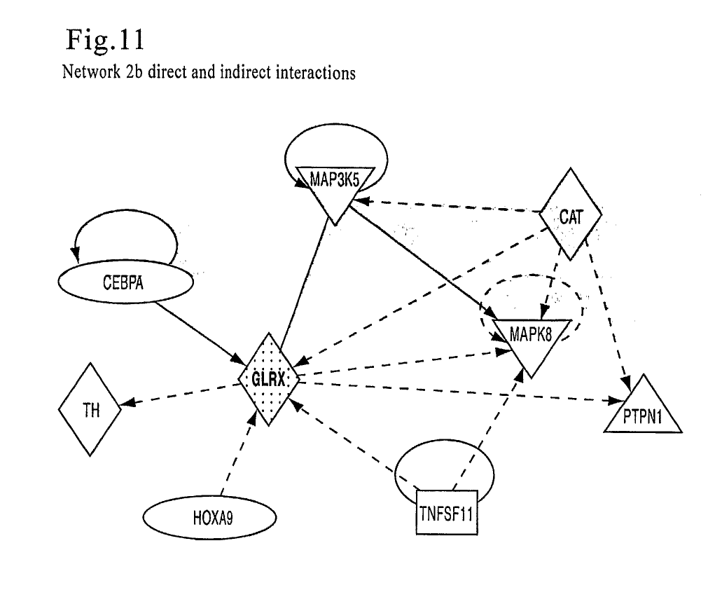

Network 2b contains 9 original nodes and includes 1 gene from the fine mapped regions (FIG. 11). For a description of the GLRX gene, please refer to the text above about network 3, direct interactions only.

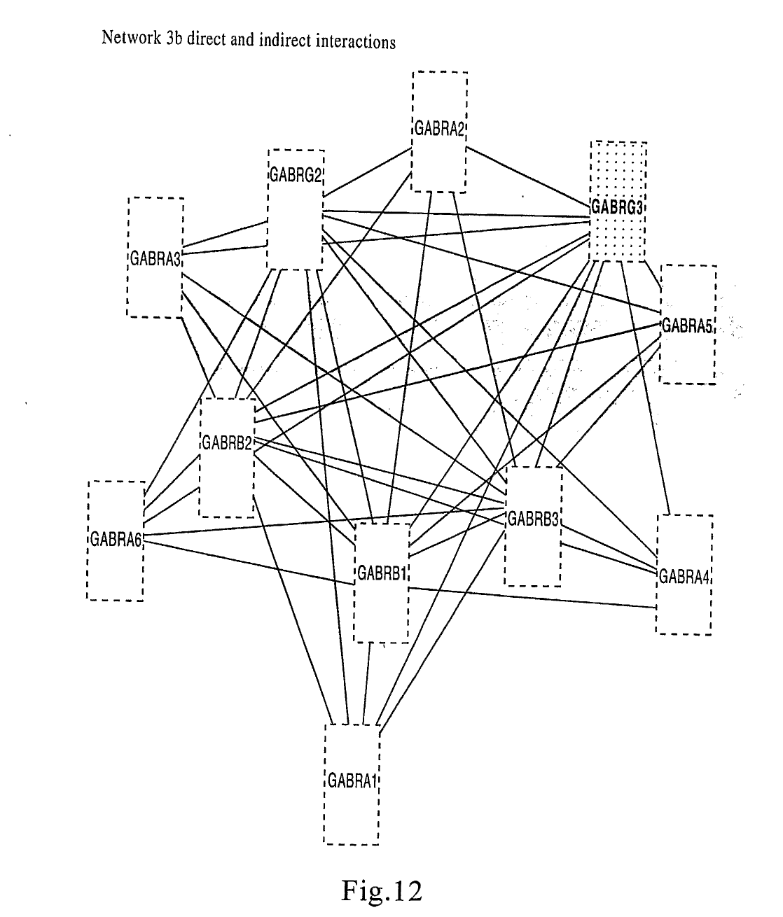

Network 3b contains 11 original nodes and includes 1 gene from the fine mapped regions (FIG. 12). For a description of the GABRG3 gene, please refer to the text above about network 5 direct interactions only.

Nucleic Acid Sequences

The nucleic acid sequences of the present invention may be derived from a variety of sources including DNA, cDNA, synthetic DNA, synthetic RNA, derivatives, mimetics or combinations thereof. Such sequences may comprise genomic DNA, which may or may not include naturally occurring introns, genic regions, nongenic regions, and regulatory regions. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means. The nucleic acids described herein are used certain embodiments of the methods of the present invention for production of RNA, proteins or polypeptides, through incorporation into cells, tissues, or organisms. In one embodiment, DNA containing all or part of the coding sequence for the genes described in Tables 10-12 and 14, or the SNP markers described in Tables 2-9 and 13, is incorporated into a vector for expression of the encoded polypeptide in suitable host cells. The invention also comprises the use of the nucleotide sequence of the nucleic acids of this invention to identify DNA probes for the genes described in Tables 10-12 and 14 or the SNP markers described in Tables 2-9 and 13, PCR primers to amplify the genes described in Tables 4-6, or the SNP markers described in Tables 2-9 and 13, nucleotide polymorphisms in the genes described in Tables 10-12 and 14, and regulatory elements of the genes described in Tables 10-12 and 14. The nucleic acids of the present invention find use as primers and templates for the recombinant production of nucleotide polymorphisms in the genes described in Tables 10-12 and 14, and regulatory elements of the genes described in Tables 10-12 and 14, or the SNP markers described in Tables 2-9 and 13. The nucleic acids of the present invention find use as primers and templates for the recombinant production of psoriasis-associated peptides or polypeptides, for chromosome and gene mapping, to provide antisense sequences, for tissue distribution studies, to locate and obtain full length genes, to identify and obtain homologous sequences (wild-type and mutants), and in diagnostic, theranostic and prognostic applications.

Antisense Oligonucleotides

In a particular embodiment of the invention, an antisense nucleic acid or oligonucleotide is wholly or partially complementary to, and can hybridize with, a target nucleic acid (either DNA or RNA) having the sequence of SEQ ID NO:1, NO:3 or any SEQ ID from Tables 2-14. For example, an antisense nucleic acid or oligonucleotide comprising 16 nucleotides can be sufficient to inhibit expression of the at least one gene from Tables 10-12 and 14. Alternatively, an antisense nucleic acid or oligonucleotide can be complementary to 5′ or 3′ untranslated regions, or can overlap the translation initiation codon (5′ untranslated and translated regions) of at least one gene from Tables 10-12 and 14, or its functional equivalent. In another embodiment, the antisense nucleic acid is wholly or partially complementary to, and can hybridize with, a target nucleic acid that encodes a polypeptide from a gene described in Tables 10-12 and 14.

In addition, oligonucleotides can be constructed which will bind to duplex nucleic acid (i.e., DNA:DNA or DNA:RNA), to form a stable triple helixcontaining or triplex nucleic acid. Such triplex oligonucleotides can inhibit transcription and/or expression of a gene from Tables 10-12 and 14, or its functional equivalent (M. D. Frank-Kamenetskii et al., 1995). Triplex oligonucleotides are constructed using the basepairing rules of triple helix formation and the nucleotide sequence of the genes described in Tables 10-12 and 14.

The present invention encompasses methods of using oligonucleotides in antisense inhibition of the function of the genes from Tables 10-12 and 14. In the context of this invention, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention. Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH3, F, OCH3, OCN, O(CH2), NH2 and O(CH2)n CH3, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with at least one gene from Tables 10-12 and 14 (DNA or RNA) to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds. Antisense nucleic acids or oligonucleotides can be produced by standard techniques (see, e.g., Shewmaker et al., U.S. Pat. No. 6,107,065. The oligonucleotides used in accordance with this invention may be conveniently and routinely made through the well-known technique of solid phase synthesis.

Any other means for such synthesis may also be employed; however, the actual synthesis of the oligonucleotides is well within the abilities of the practitioner. It is also well known to prepare other oligonucleotides such as phosphorothioates and alkylated derivatives.

The oligonucleotides of this invention are designed to be hybridizable with RNA (e.g., mRNA) or DNA from genes described in Tables 10-12 and 14. For example, an oligonucleotide (e.g., DNA oligonucleotide) that hybridizes to mRNA from a gene described in Tables 10-12 and 14 can be used to target the mRNA for RnaseH digestion. Alternatively; an oligonucleotide that can hybridize to the translation initiation site of the mRNA of a gene described in Tables 10-12 and 14 can be used to prevent translation of the mRNA. In another approach, oligonucleotides that bind to the double-stranded DNA of a gene from Tables 10-12 and 14 can be administered. Such oligonucleotides can form a triplex construct and inhibit the transcription of the DNA encoding polypeptides of the genes described in Tables 10-12 and 14. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may he targeted to hybridize to the following regions: mRNA cap region; translation initiation site; translational termination site; transcription initiation site; transcription termination site; polyadenylation signal; 3′ untranslated region; 5′ untranslated region; 5′coding region; mid coding region; and 3′coding region. Preferably, the complementary oligonucleotide is designed to hybridize to the most unique 5′ sequence of a gene described in Tables 10-12 and 14, including any of about 15-35 nucleotides spanning the 5′ coding sequence. In accordance with the present invention, the antisense oligonucleotide can be synthesized, formulated as a pharmaceutical composition, and administered to a subject. The synthesis and utilization of antisense and triplex oligonucleotides have been previously described (e.g., Simon et al., 1999; Bane et al., 2000; Elez et al., 2000; Sauter et al., 2000).

Alternatively, expression vectors derived from retroviruses, adenovirus, herpes or vaccinia viruses, or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a polypeptide from the genes described in Tables 10-12 and 14. These techniques are described both in Sambrook et al., 1989 and in Ausubel et al., 1992. For example, expression of at least one gene from Tables 10-12 can be inhibited by transforming a cell or tissue with an expression vector that expresses high levels of untranslatable sense or antisense sequences. Even in the absence of integration into the DNA, such vectors may continue to transcribe RNA molecules until they are disabled by endogenous nucleases. Transient expression may last for a month or more with a nonreplicating vector, and even longer if appropriate replication elements are included in the vector system. Various assays may be used to test the ability of gene-specific antisense oligonucleotides to inhibit the expression of at least one gene from Tables 10-12 and 14. For example, mRNA levels of the genes described in Tables 10-12 and 14 can be assessed by Northern blot analysis (Sambrook et al., 1989; Ausubel et al., 1992; J. C. Alwine et al. 1977; I. M. Bird, 1998), quantitative or semi-quantitative RT-PCR analysis (see, e.g., W. M. Freeman et al., 1999; Ren et al., 1998; J. M. Cale et al., 1998), or in situ hybridization (reviewed by A. K. Raap, 1998). Alternatively, antisense oligonucleotides may be assessed by measuring levels of the polypeptide from the genes described in Tables 10-12, and 14, e.g., by western blot analysis, indirect immunofluorescence and immunoprecipitation techniques (see, e.g., J. M. Walker, 1998, Protein Protocols on CD-ROM, Humana Press, Totowa, N.J.). Any other means for such detection may also be employed, and are well within the abilities of the practitioner.

Mapping Technologies

The present invention includes various methods which employ mapping technologies to map SNPs and polymorphisms. For purpose of clarity, this section comprises, but is not limited to, the description of mapping technologies that can be utilized to achieve the embodiments described herein. Mapping technologies may be based on amplification methods, restriction enzyme cleavage methods, hybridization methods, sequencing methods, and cleavage methods using agents.

Amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002; and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

Restriction enzyme cleavage methods include: isolating sample and control DNA, amplification (optional), digestion with one or more restriction endonucleases, determination of fragment length sizes by gel electrophoresis and comparing samples and controls. Differences in fragment length sizes between sample and control DNA indicates mutations in the sample DNA. Moreover, sequence specific ribozymes (see, e.g., U.S. Pat. No. 5,498,531) or DNAzyme (e.g. U.S. Pat. No. 5,807,718) can be used to score for the presence of specific mutations by development or loss of a ribozyme or DNAzyme cleavage site.

SNPs and SNP maps of the invention can be identified or generated by hybridizing sample nucleic acids, e.g., DNA or RNA, to high density arrays or bead arrays containing oligonucleotide probes corresponding to the polymorphisms of Tables 2-9 and 13 (see the Affymetrix arrays and Illumina bead sets at www.affymetrix.com and www.illumina.com and see Cronin et al., 1996; or Kozal et al., 1996).

A variety of sequencing reactions known in the art can be used to directly sequence nucleic acids for the presence or the absence of one or more polymorphisms of Tables 2-9 and 13. Examples of sequencing reactions include those based on techniques developed by Maxam and Gilbert (1977) or Sanger (1977). It is also contemplated that any of a variety of automated sequencing procedures can be utilized, including sequencing by mass spectrometry (see, e.g. PCT International Publication No. WO 94/16101; Cohen et al., 1996; and Griffin et al., 1993), real-time pyrophosphate sequencing method (Ronaghi et al., 1998; and Permutt et al., 2001) and sequencing by hybridization (see e.g. Drmanac et al., 2002).

Other methods of detecting polymorphisms include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing a wild-type sequence with potentially mutant RNA or DNA obtained from a sample. The double-stranded duplexes are treated with an agent who cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of a mutation or SNP (see, for example, Cotton et al., 1988; and Saleeba et al., 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping polymorphism. For example, the mutY enzyme of E. coli cleaves A at G/A mismatches (Hsu et al., 1994). Other examples include, but are not limited to, the MutHLS enzyme complex of E. coli (Smith and Modrich Proc. 1996) and Cel 1 from the celery (Kulinski et al., 2000) both cleave the DNA at various mismatches. According to an exemplary embodiment, a probe based on a polymorphic site corresponding to a polymorphism of Tables 10-12 and 14 is hybridized to a cDNA or other DNA product from a test cell or cells. The duplex is treated with a DNA mismatch repair enzyme, and the cleavage products, if any, can be detected from electrophoresis protocols or the like (see, for example, U.S. Pat. No. 5,459,039). Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify polymorphisms in a sample. For example, single strand conformation polymorphism (SSCP) analysis can be used to detect differences in electrophoretic mobility between mutant and wild type nucleic acids (Orita et al., 1989; Cotton et al., 1993; and Hayashi 1992). Single-stranded DNA fragments of case and control nucleic acids will be denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting polymorphism include, but are not limited to, selective oligonucleotide hybridization, selective amplification, selective primer extension, selective ligation, single-base extension, selective termination of extension or invasive cleavage assay. For example, oligonucleotide primers may be prepared in which the polymorphism is placed centrally and then hybridized to target DNA under conditions which permit hybridization only if a perfect match is found (Saiki et al., 1986); Saiki et al., 1989). Such allele specific oligonucleotides are hybridized to PCR amplified target DNA or a number of different mutations when the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

Hybridization assays may also be carried out with a temperature gradient following the principle of dynamic allele-specific hybridization or like e.g. Jobs et al., (2003); and Bourgeois and Labuda, (2004). For example, the hybridization is done using one of the two allele-specific oligonucleotides labeled with a fluorescent dye, an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of an associated allele, a particular allele of a polymorphic locus, or the like (e.g. Jobs et al., 2003). Alternatively, the hybridization is done under a gradually decreasing temperature. In this case, both allele-specific oligonucleotides are hybridized to the template competitively. At high temperature none of the two probes is hybridized. Once the optimal temperature of the full-matched probe is reached, it hybridizes and leaves no target for the mismatched probe (e.g. Bourgeois and Labuda, 2004). In the latter case, if the allele-specific probes are differently labeled, then they are hybridized to a single PCR-amplified target. If the probes are labeled with the same dye, then the probe cocktail is hybridized twice to identical templates with only one labeled probes, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, a particular allele of a polymorphic locus, or the like, also referred to as “mutation” of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the mutation to create cleavage-based detection (Gasparini et al., 1992)). It is anticipated that in certain embodiments amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., (1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNPs and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78 (2003)).

Another alternative includes the single-base extension/ligation assay using a molecular inversion probe, consisting of a single, long oligonucleotide (see e.g. Hardenbol et al., 2003). In such an embodiment, the oligonucleotide hybridizes on both side of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, and bead array or like. This reaction can be performed at a high multiplexed level.

In another alternative, the associated allele, a particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, and bead array or like. In the latter case, the extension oligonucleotide will contain a SNP-specific zip code tag.

In yet another alternative, a SNP is scored by selective termination of extension. The template is first amplified by PCR and the extension oligonucleotide hybridizes in vicinity to the SNP locus, close to but not necessarily adjacent to it. The extension reaction is carried out using a thermostable polymerase such as Thermo Sequenase (GE Healthcare) in the presence of a mix of dNTPs and at least one ddNTP. The latter has to terminate the extension at one of the allele of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, SNPs are detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Methods to Identify Agents that Modulate the Expression of a Nucleic Acid Encoding a Gene Involved in Psoriasis.

The present invention provides methods for identifying agents that modulate the expression of a nucleic acid encoding a gene from Tables 10-12 and 14. Such methods may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Such cells can be obtained from any parts of the body such as the scalp, blood, dermis, epidermis and other skin cells, cutaneous surfaces, intertrigious areas, genitalia, vessels and endothelium. Some non-limiting examples of cells that can be used are keratinocytes, monocytes, neutrophils, langerhans cells, CD4+ and CD8+ T cells and lymphocytes. Cytokines and lymphokines can also be used.

In one assay format, the expression of a nucleic acid encoding a gene of the invention (see Tables 10-12 and 14) in a cell or tissue sample is monitored directly by hybridization to the nucleic acids of the invention. Cell lines or tissues are exposed to the agent to be tested under appropriate conditions and time and total RNA or mRNA is isolated by standard procedures such as those disclosed in Sambrook et al., (1989) Molecular Cloning: A Laboratory Manual, Cold Spring Harbor Laboratory Press).

Probes to detect differences in RNA expression levels between cells exposed to the agent and control cells may be prepared as described above. Hybridization conditions are modified using known methods, such as those described by Sambrook et al., and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA RNA from a sample under conditions in which the affixed sequences will specifically hybridize to the RNA. By examining for the ability of a given probe to specifically hybridize to an RNA sample from an untreated cell population and from a cell population exposed to the agent, agents which up or down regulate expression are identified.

Methods to Identify Agents that Modulate the Activity of a Protein Encoded by a Gene Involved in Psoriasis.

The present invention provides methods for identifying agents that modulate at least one activity of the proteins described in Tables 10-12 and 14. Such methods may utilize any means of monitoring or detecting the desired activity. As used herein, an agent is said to modulate the expression of a protein of the invention if it is capable of up- or down-regulating expression of the protein in a cell. Such cells can be obtained from any parts of the body such as the scalp, blood, dermis, epidermis and other skin cells, cutaneous surfaces, intertrigious areas, genitalia, vessels and endothelium. Some non-limiting examples of cells that can be used are keratinocytes, monocytes, neutrophils, langerhans cells, CD4+ and CD8+ T cells and lymphocytes. Cytokines and lymphokines can also be used.

In one format, the specific activity of a protein of the invention, normalized to a standard unit, may be assayed in a cell population that has been exposed to the agent to be tested and compared to an unexposed control cell population may be assayed. Cell lines or populations are exposed to the agent to be tested under appropriate conditions and times. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) or modifications which affect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies may be recovered from the culture supernatant or from the ascites supernatant. Fragments of the monoclonal antibodies or the polyclonal antisera which contain the immunologically significant portion(s) can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin. The antibodies or fragments may also be produced, using current technology, by recombinant means. Antibody regions that bind specifically to the desired regions of the protein can also be produced in the context of chimeras derived from multiple species. Antibody regions that bind specifically to the desired regions of the protein can also be produced in the context of chimeras from multiple species, for instance, humanized antibodies. The antibody can therefore be a humanized antibody or a human antibody, as described in U.S. Pat. No. 5,585,089 or Riechmann et al. (1988).

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use of a chemical library or a peptide combinatorial library, or a growth broth of an organism. As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. The agents of the present invention can be, as examples, oligonucleotides, antisense polynucleotides, interfering RNA, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention includes antibodies or fragments thereof that bind to a protein encoded by a gene in Tables 10-12, and 14. Antibody agents can be obtained by immunization of suitable mammalian subjects with peptides, containing as antigenic regions, those portions of the protein intended to be targeted by the antibodies (see section above of antibodies as probes for standard antibody preparation methodologies).

In yet another class of agents, the present invention includes peptide mimetics that mimic the three-dimensional structure of the protein encoded by a gene from Tables 10-12, and 14. Such peptide mimetics may have significant advantages over naturally-occurring naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly used in the pharmaceutical industry as non-peptide drugs with properties analogous to those of the template peptide. These types of non-peptide compounds are also referred to as peptide mimetics or peptidomimetics (Fauchere, 1986; Veber & Freidinger, 1985; Evans et al., 1987) which are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage using methods known in the art. Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) to which the peptide mimetic binds to produce the therapeutic effect. Derivitization (e.g., labeling) of peptide mimetics should not substantially interfere with the desired biological or pharmacological activity of the peptide mimetic. The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., 1991) and the phage display method. The two hybrid method detects protein-protein interactions in yeast (Fields et al., 1989). The phage display method detects the interaction between an immobilized protein and a protein that is expressed on the surface of phages such as lambda and M13 (Amberg et al., 1993; Hogrefe et al., 1993). These methods allow positive and negative selection for protein-protein interactions and the identification of the sequences that determine these interactions.

Method to Diagnose Psoriasis

The present invention also relates to methods for diagnosing inflammatory disease or a related disease, preferably psoriasis, a disposition to such disease, predisposition to such a disease and/or disease progression. In some methods, the steps comprise contacting a target sample with (a) nucleic acid molecule(s) or fragments thereof and comparing the concentration of individual mRNA(s) with the concentration of the corresponding mRNA(s) from at least one healthy donor. An aberrant (increased or decreased) mRNA level of at least one gene from Tables 10-12 and 14, at least 5 or 10 genes from Tables 10-12 and 14, at least 50 genes from Tables 10-12 and 14, at least 100 genes from Tables 10-12 and 14 or at least 200 genes from Tables 10-12 and 14 determined in the sample in comparison to the control sample is an indication of psoriasis or a related disease or a disposition to such kinds of diseases. For diagnosis, samples are, preferably, obtained from inflamed tissues. Samples can also be obtained from any parts of the body such as the scalp, blood, dermis, epidermis and other skin cells, cutaneous surfaces, intertrigious areas, genitalia, vessels and endothelium. Some non-limiting examples of cells that can be used are keratinocytes, monocytes, neutrophils, langerhans cells, CD4+ and CD8+ T cells and lymphocytes. Cytokines and lymphokines can also be used.

For analysis of gene expression, total RNA is obtained from cells according to standard procedures and, preferably, reverse-transcribed. Preferably, a DNAse treatment (in order to get rid of contaminating genomic DNA) is performed. Some non-limiting examples of cells that can be used are: keratinocytes, monocytes, neutrophils, langerhans cells, CD4+ and CD8+ T cells and lymphocytes. Cytokines and lymphokines can also be used.

The nucleic acid molecule or fragment is typically a nucleic acid probe for hybridization or a primer for PCR. The person skilled in the art is in a position to design suitable nucleic acids probes based on the information provided in the Tables of the present invention. The target cellular component, i.e. mRNA, e.g., in skin, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP-B10237362) and other detection assays that are known to those skilled in the art. Products obtained by in vitro amplification can be detected according to established methods, e.g. by separating the products on agarose or polyacrylamide gels and by subsequent staining with ethidium bromide. Alternatively, the amplified products can be detected by using labeled primers for amplification or labeled dNTPs. Preferably, detection is based on a microarray.

The probes (or primers) (or, alternatively, the reverse-transcribed sample mRNAs) can be detectably labeled, for example, with a radioisotope, a bioluminescent compound, a chemiluminescent compound, a fluorescent compound, a metal chelate, or an enzyme.

The present invention also relates to the use of the nucleic acid molecules or fragments described above for the preparation of a diagnostic composition for the diagnosis of psoriasis or a disposition to such a disease.

The present invention also relates to the use of the nucleic acid molecules of the present invention for the isolation or development of a compound which is useful for therapy of psoriasis. For example, the nucleic acid molecules of the invention and the data obtained using said nucleic acid molecules for diagnosis of psoriasis might allow for the identification of further genes which are specifically dysregulated, and thus may be considered as potential targets for therapeutic interventions.

The invention further provides prognostic assays that can be used to identify subjects having or at risk of developing psoriasis. In such method, a test sample is obtained from a subject and the amount and/or concentration of the nucleic acid described in Tables 10-12 and 14 is determined; wherein the presence of an associated allele, a particular allele of a polymorphic locus, or the likes in the nucleic acids sequences of this invention (see SEQ ID from Tables 2-14) can be diagnostic for a subject having or at risk of developing psoriasis. As used herein, a “test sample” refers to a biological sample obtained from a subject of interest. For example, a test sample can be a biological fluid, a cell sample, or tissue. A biological fluid can be, but is not limited to saliva, serum, mucus, urine, stools spermatozoids, vaginal secretions, lymph, amiotic liquid, pleural liquid and tears. Cells can be, but are not limited keratinocytes, monocytes, neutrophils, langerhans cells, CD4+ and CD8+ T cells and lymphocytes. Cytokines and lymphokines can also be used.

Furthermore, the prognostic assays described herein can be used to determine whether a subject can be administered an agent (e.g., an agonist, antagonist, peptidomimetic, polypeptide, nucleic acid such as antisense DNA or interfering RNA (RNAi), small molecule or other drug candidate) to treat psoriasis. Specifically, these assays can be used to predict whether an individual will have an efficacious response or will experience adverse events in response to such an agent. For example, such methods can be used to determine whether a subject can be effectively treated with an agent that modulates the expression and/or activity of a gene from Tables 10-12 and 14, polymorphisms from Tables 2-9 and 13, or the nucleic acids described herein. In another example, an association study may be performed to identify polymorphisms from Tables 2-14 that are associated with a given response to the agent, e.g., an efficacious response or the likelihood of one or more adverse events. Thus, the present invention provides methods for determining whether a subject can be effectively treated with an agent for a disorder associated with aberrant expression or activity of a gene from Tables 10-12 and 14 or polymorphisms from Tables 2-9 and 13, in which a test sample is obtained and nucleic acids or polypeptides from Tables 10-12 and 14 are detected (e.g., wherein the presence of a particular level of expression of a gene from Tables 10-12 and 14 or a particular allelic variant of such gene, such as polymorphism from Tables 2-9 and 13, is diagnostic for a subject that can be administered an agent to treat a disorder such as psoriasis). In the one embodiment, the method includes obtaining a sample from a subject suspected of having psoriasis or an affected individual and exposing such sample to an agent. The expression and/or activity of the nucleic acids and or genes of the invention is monitored before and after treatment with such agent to assess the effect of such agent. After analysis of the expression values, one skilled in the art can determine whether such agent can effectively treat such subject. In another embodiment, the method includes obtaining a sample from a subject having or susceptible to developing psoriasis and determining the allelic constitution of one or more polymorphisms from Tables 2-14, which are associated with a particular response to an agent. After analysis of the allelic constitution of the individual at the associated polymorphisms (e.g., genotyping), one skilled in the art can determine whether such agent can effectively treat such subject.

The methods of the invention can also be used to detect genetic alterations in a gene from Tables 10-12 and 14, thereby determining if a subject with the lesioned gene is at risk for a disorder associated with psoriasis. In preferred embodiments, the methods include detecting, in a sample of cells from the subject, the presence or absence of a genetic alteration characterized by at least one alteration linked to or affecting the integrity of a gene from Tables 10-12 and 14 encoding a polypeptide or the misexpression of such gene. For example, such genetic alterations can be detected by ascertaining the existence of at least one of: (1) a deletion of one or more nucleotides from a gene from Tables 10-12 and 14; (2) an addition of one or more nucleotides to a gene from Tables 10-12 and 14; (3) a substitution of one or more nucleotides of a gene from Tables 10-12 and 14; (4) a chromosomal rearrangement of a gene from Tables 10-12 and 14; (5) an alteration in the level of a messenger RNA transcript of a gene from Tables 10-12 and 14; (6) aberrant modification of a gene from Tables 10-12 and 14, such as of the methylation pattern of the genomic DNA, (7) the presence of a non-wild type splicing pattern of a messenger RNA transcript of a gene from Tables 10-12 and 14; (8) inappropriate post-translational modification of a polypeptide encoded by a gene from Tables 10-12 and 14; and (9) alternative promoter use. As described herein, there are a large number of assay techniques known in the art which can be used for detecting alterations in a gene from Tables 10-12 and 14. A preferred biological sample is a peripheral blood sample obtained by conventional means from a subject. Another preferred biological sample is a buccal swab. Other biological samples can be, but are not limited to, urine, stools spermatozoids, vaginal secretions, lymph, amiotic liquid, pleural liquid and tears.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or alternatively, in a ligation chain reaction (LCR) (see, e.g., Landegran et al., 1988; and Nakazawa et al., 1994), the latter of which can be particularly useful for detecting point mutations in a gene from Tables 10-12 and 14 (see Abavaya et al., 1995). This method can include the steps of collecting a sample of cells from a patient, isolating nucleic acid (e.g., genomic DNA, mRNA, or both) from the cells of the sample, contacting the nucleic acid sample with one or more primers which specifically hybridize to a gene from Tables 10-12 and 14 under conditions such that hybridization and amplification of the nucleic acid from Tables 10-12 and 14 (if present) occurs, and detecting the presence or absence of an amplification product, or detecting the size of the amplification product and comparing the length to a control sample. PCR and/or LCR may be desirable to use as a preliminary amplification step in conjunction with some of the techniques used for detecting a mutation, an associated allele, a particular allele of a polymorphic locus, or the like, described herein.

Alternative amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002); and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

In an alternative embodiment, alterations in a gene from Tables 10-12 and 14, from a sample cell can be identified by identifying changes in a restriction enzyme cleavage pattern. For example, sample and control DNA is isolated, amplified (optionally), digested with one or more restriction endonucleases, and fragment length sizes are determined by gel electrophoresis and compared. Differences in fragment length sizes between sample and control DNA indicate a mutation(s), an associated allele, a particular allele of a polymorphic locus, or the like, in the sample DNA. Moreover, sequence specific ribozymes (see, e.g., U.S. Pat. No. 5,498,531 or DNAzyme e.g. U.S. Pat. No. 5,807,718) can be used to score for the presence of specific associated allele, a particular allele of a polymorphic locus, or the likes by development or loss of a ribozyme or DNAzyme cleavage site.

The present invention also relates to further methods for diagnosing inflammatory disease or a related disease, preferably psoriasis, a disposition to such disease, predisposition to such a disease and/or disease progression. In some methods, the steps comprise contacting a target sample with (a) nucleic molecule(s) or fragments thereof and determining the presence or absence of a particular allele of a polymorphism that confers a disease-related phenotype (e.g., predisposition to such a disease and/or disease progression). The presence of at least one allele from Tables 2-9 and 13 that is associated with psoriasis disease (“associated allele”), at least 5 or 10 associated alleles from Tables 2-9 and 13, at least 50 associated alleles from Tables 2-9 and 13, at least 100 associated alleles from Tables 2-9 and 13, or at least 200 associated alleles from Tables 2-9 and 13 determined in the sample is an indication of psoriasis or a related disease, a disposition or predisposition to such kinds of diseases, or a prognosis for such disease progression. Samples may be obtained from any parts of the body such as the scalp, blood, dermis, epidermis and other skin cells, cutaneous surfaces, intertrigious areas, genitalia, vessels and endothelium. Some non-limiting examples of cells that can be used are keratinocytes, cytokines, neutrophils, langerhans cells, CD4+ and CD8+ T cells and lymphocytes. Lymphokines and monocytes can also be used.

In other embodiments, alterations in a gene from Tables 10-12 and 14 or a locus from Table 1 or different alleles of the polymorphisms in Tables 2-9 and 13 can be identified by hybridizing a sample and control nucleic acids, e.g., DNA or RNA, to high density arrays or bead arrays containing tens to thousands of oligonucleotide probes (Cronin et al., 1996; Kozal et al., 1996). For example, alterations in a gene from Tables 10-12 and 14 or a locus from Table 1, or different alleles of the polymorphisms from Tables 2-9 and 13 can be identified in two dimensional arrays containing light-generated DNA probes as described in Cronin et al., (1996). Briefly, a first hybridization array of probes can be used to scan through long stretches of DNA in a sample and control to identify base changes between the sequences by making linear arrays of sequential overlapping probes. This step allows the identification of point mutations or different alleles of polymorphisms. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene or associated alleles or particular allele of a polymorphic locus.

In yet another embodiment, any of a variety of sequencing reactions known in the art can be used to directly sequence a gene from Tables 10-12 and 14 and detect an associated allele, a particular allele of a polymorphic locus, or the like by comparing the sequence of the sample gene from Tables 10-12 and 14 with the corresponding wild-type (control) sequence. Examples of sequencing reactions include those based on techniques developed by Maxam and Gilbert (1977) or Sanger (1977). It is also contemplated that any of a variety of automated sequencing procedures can be utilized when performing the diagnostic assays {Bio/Techniques 19:448 (1995) including sequencing by mass spectrometry (see, e.g. PCT International Publication No. WO 94/16101; Cohen et al., 1996; and Griffin et al. 1993), real-time pyrophosphate sequencing method (Ronaghi et al., 1998; and Permutt et al., 2001) and sequencing by hybridization (see e.g. Drmanac et al., 2002)}.

Other methods of detecting an associated allele, a particular allele of a polymorphic locus, or the likes in a gene from Tables 10-12 and 14 include methods in which protection from cleavage agents is used to detect mismatched bases in RNA/RNA, DNA/DNA or RNA/DNA heteroduplexes (Myers et al., 1985). In general, the art technique of “mismatch cleavage” starts by providing heteroduplexes formed by hybridizing (labeled) RNA or DNA containing the wild-type gene from Tables 10-12 and 14 sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of an associated allele, a particular allele of a polymorphic locus, or the like (see, for example, Cotton et al., 1988; Saleeba et al., 1992). In a preferred embodiment, the control DNA or RNA can be labeled for detection, as described herein.

In still another embodiment, the mismatch cleavage reaction employs one or more proteins that recognize mismatched base pairs in double-stranded DNA (so called “DNA mismatch repair” enzymes) in defined systems for detecting and mapping point an associated allele, a particular allele of a polymorphic locus, or the likes in a gene from Tables 10-12 and 14 cDNAs obtained from samples of cells. For example, the mutY enzyme of E. coli cleaves A at G/A mismatches (Hsu et al., 1994). Other examples include, but are not limited to, the MutHLS enzyme complex of E. coli (Smith and Modrich., 1996) and Cel 1 from the celery (Kulinski et al., 2000) both cleave the DNA at various mismatches. According to an exemplary embodiment, a probe based on a gene sequence from Tables 10-12 and 14 is hybridized to a cDNA or other DNA product from a test cell or cells. The duplex is treated with a DNA mismatch repair enzyme, and the cleavage products, if any, can be detected using electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify an associated allele, a particular allele of a polymorphic locus, or the likes in genes from Tables 10-12 and 14. For example, single strand conformation polymorphism (SSCP) can be used to detect differences in electrophoretic mobility between mutant and wild type nucleic acids (Orita et al., 1993; see also Cotton, 1993; and Hayashi et al., 1992). Single-stranded DNA fragments of sample and control nucleic acids from Tables 10-12 and 14 will be denatured and allowed to renature. The secondary structure of single-stranded nucleic acids varies according to sequence; the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting point mutations an associated allele, a particular allele of a polymorphic locus, or the like include, but are not limited to, selective oligonucleotide hybridization, selective amplification, selective primer extension, selective ligation, single-base extension, selective termination of extension or invasive cleavage assay. For example, oligonucleotide primers may be prepared in which the known associated allele, particular allele of a polymorphic locus, or the like is placed centrally and then hybridized to target DNA under conditions which permit hybridization only if a perfect match is found (Saiki et al., 1986; Saiki et al., 1989). Such allele specific oligonucleotides are hybridized to PCR amplified target DNA or a number of different associated alleles, a particular allele of a polymorphic locus, or the likes where the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like locus is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

The hybridization may also be carried out with a temperature gradient following the principle of dynamic allele-specific hybridization or like (e.g. Jobs et al., 2003); and Bourgeois and Labuda, 2004). For example, the hybridization is done using one of the two allele-specific oligonucleotides labeled with a fluorescent dye, an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of the associated allele, particular allele of a polymorphic locus, or the like (e.g. Jobs et al. supra). Alternatively, the hybridization is done under a gradually decreasing temperature. In this case, both allele-specific oligonucleotides are hybridized to the template competitively. At high temperature none of the two probes is hybridized. Once the optimal temperature of the full-matched probe is reached, it hybridizes and leaves no target for the mismatched probe. In the latter case, if the allele-specific probes are differently labeled, then they are hybridized to a single PCR-amplified target. If the probes are labeled with the same dye, then the probe cocktail is hybridized twice to identical templates with only one labeled probes, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, particular allele of a polymorphic locus, or the like of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the associated allele, particular allele of a polymorphic locus, or the like to create cleavage-based detection (Gasparini et al., 1992). It is anticipated that in certain embodiments amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNP and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78 (2003)).

Another alternative includes the single-base extension/ligation assay using a molecular inversion probe, consisting of a single, long oligonucleotide (see e.g. Hardenbol et al., 2003). In such an embodiment, the oligonucleotide hybridizes on both sides of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, bead array or like. This reaction can be performed at a highly multiplexed level.

In another alternative, the associated allele, particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, bead array or the like. In the latter case, the extension oligonucleotide will contain a SNP-specific zip code tag.

In yet another alternative, the variant is scored by selective termination of extension. The template is first amplified by PCR and the extension oligonucleotide hybridizes in vicinity to the SNP locus, close to but not necessarily adjacent to it. The extension reaction is carried out using a thermostable polymerase such as Thermo Sequenase (GE Healthcare) in the presence of a mix of dNTPs and at least one ddNTP. The latter has to terminate the extension at one of the alleles of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, the associated allele, particular allele of a polymorphic locus, or the like is detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Other types of markers can also be used for diagnostic purposes. For example, microsatellites can also be useful to detect the genetic predisposition of an individual to a given disease. Microsatellites consist of short sequence motifs of one or a few nucleotides repeated in tandem. The most common motifs are polynucleotide runs, dinucleotide repeats (particularly the CA repeats) and trinucleotide repeats. However, other types of repeats can also be used. The microsatellites are very useful for genetic mapping because they are highly polymorphic in their length. Microsatellite markers can be typed by various means, including but not limited to DNA PCR fragment sizing, oligonucleotide ligation assay and mass spectrometry. For example, the locus of the microsatellite is amplified by PCR and the size of the PCR fragment will be directly correlated to the length of the microsatellite repeat. The size of the PCR fragment can be detected by regular means of gel electrophoresis. The fragment can be labeled internally during PCR or by using end-labeled oligonucleotides in the PCR reaction (e.g. Mansfield et al., 1996). Alternatively, the size of the PCR fragment is determined by mass spectrometry. In such a case, however, the flanking sequences need to be eliminated. This can be achieved by ribozyme cleavage of an RNA transcript of the microsatellite repeat (Krebs et al., 2001). For example, the microsatellite locus is amplified using oligonucleotides that include a T7 promoter on one end and a ribozyme motif on the other end. Transcription of the amplified fragments will yield an RNA substrate for the ribozyme, releasing small RNA fragments that contain the repeated region. The size of the latter is determined by mass spectrometry. Alternatively, the flanking sequences are specifically degraded. This is achieved by replacing the dTTP in the PCR reaction by dUTP. The dUTP nucleosides are then removed by uracyl DNA glycosylases and the resulting abasic sites are cleaved by either abasic endonucleases such as human AP endonuclease or chemical agents such as piperidine. Bases can also be modified post-PCR by chemical agents such as dimethyl sulfate and then cleaved by other chemical agents such as piperidine (see e.g. Maxam and Gilbert, 1977; U.S. Pat. No. 5,869,242; and U.S. Patent pending Ser. No. 60/335,068).

In another alternative, an oligonucleotide ligation assay can be performed. The microsatellite locus is first amplified by PCR. Then, different oligonucleotides can be submitted to ligation at the center of the repeat with a set of oligonucleotides covering all the possible lengths of the marker at a given locus (Zirvi et al., 1999). Another example of design of an oligonucleotide assay comprises the ligation of three oligonucleotides; a 5′ oligonucleotide hybridizing to the 5′ flanking sequence, a repeat oligonucleotide of the length of the shortest allele of the marker hybridizing to the repeated region and a set of 3′ oligonucleotides covering all the existing alleles hybridizing to the 3′ flanking sequence and a portion of the repeated region for all the alleles longer than the shortest one. For the shortest allele, the 3′ oligonucleotide exclusively hybridizes to the 3′ flanking sequence (U.S. Pat. No. 6,479,244).

The methods described herein may be performed, for example, by utilizing pre-packaged diagnostic kits comprising at least one probe nucleic acid selected from the SEQ ID of Tables 2-14, or antibody reagent described herein, which may be conveniently used, for example, in a clinical setting to diagnose patient exhibiting symptoms or a family history of a disease or disorder involving abnormal activity of genes from Tables 10-12 and 14.

Method to Treat an Animal Suspected of Having Psoriasis

The present invention provides methods of treating a disease associated with psoriasis by expressing in vivo the nucleic acids of at least one gene from Tables 10-12 and 14. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids encoding a gene from Tables 10-12 and 14, under the control of a promoter, then expresses the encoded protein, thereby mitigating the effects of absent, partial inactivation, or abnormal expression of a gene from Tables 10-12 and 14.

Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see Anderson, 1992; Nabel & Feigner, 1993; Mitani & Caskey, 1993; Mulligan, 1993; Dillon, 1993; Miller, 1992; Van Brunt, 1998; Vigne, 1995; Kremer & Perricaudet 1995; Doerfler & Bohm 1995; and Yu et al., 1994).

Delivery of the gene or genetic material into the cell is the first critical step in gene therapy treatment of disease. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see the references included in the above section.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992; Johann et al., 1992; Sommerfelt et al., 1990; Wilson et al., 1989; Miller et al., 1999; and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987; U.S. Pat. No. 4,797,368; WO 93/24641; Kotin, 1994; Muzyczka, 1994). Construction of recombinant AAV vectors are described in a number of publications, including U.S. Pat. No. 5,173,414; Tratschin et al., 1985; Tratschin, et al., 1984; Hermonat & Muzyczka, 1984; and Samulski et al., 1989.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors (Ellem et al., 1997; and Dranoff et al., 1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 by inverted terminal repeats flanking the transgene expression cassette. Efficient gene transfer and stable transgene delivery due to integration into the genomes of the transduced cell are key features for this vector system. (Wagner et al., 1998, Kearns et al 1996).

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., 1998). Additional examples of the use of adenovirus vectors for gene transfer in clinical trials include Rosenecker et al., 1996; Sterman et al., 1998; Welsh et al., 1995; Alvarez et al., 1997; Topf et al., 1998; Sterman et al., 1998.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., 1994; and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells, as described above. The nucleic acids from Tables 10-12 and 14 are administered in any suitable manner, preferably with the pharmaceutically acceptable carriers described above. Suitable methods of administering such nucleic acids are available and well known to those of skill in the art, and, although more than one route can be used to administer a particular composition, a particular route can often provide a more immediate and more effective reaction than another route (see Samulski et al., 1989). The present invention is not limited to any method of administering such nucleic acids, but preferentially uses the methods described herein.

The present invention further provides other methods of treating psoriasis such as administering to an individual having psoriasis an effective amount of an agent that regulates the expression, activity or physical state of at least one gene from Tables 10-12 and 14. An “effective amount” of an agent is an amount that modulates a level of expression or activity of a gene from Tables 10-12 and 14, in a cell in the individual at least about 10%, at least about 20%, at least about 30%, at least about 40%, at least about 50%, at least about 60%, at least about 70%, at least about 80% or more, compared to a level of the respective gene from Tables 10-12 and 14 in a cell in the individual in the absence of the compound. The preventive or therapeutic agents of the present invention may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen. The therapeutic efficacy of the treatment may be monitored by observing various parts of the body, such as the skin, by any monitoring method known in the art. Others ways of monitoring efficacy can be, but are not limited to monitoring inflammatory condition of the skin.

The present invention further provides a method of treating an individual clinically diagnosed with psoriasis. The methods generally comprises analyzing a biological sample that includes a cell, in some cases, a skin cell, from an individual clinically diagnosed with psoriasis for the presence of modified levels of expression of at least 1 gene, at least 10 genes, at least 50 genes, at least 100 genes, or at least 200 genes from Tables 10-12 and 14. A treatment plan that is most effective for individuals clinically diagnosed as having a condition associated with psoriasis is then selected on the basis of the detected expression of such genes in a cell. Treatment may include administering a composition that includes an agent that modulates the expression or activity of a protein from Tables 10-12 and 14 in the cell. Information obtained as described in the methods above can also be used to predict the response of the individual to a particular agent. Thus, the invention further provides a method for predicting a patient's likelihood to respond to a drug treatment for a condition associated with psoriasis, comprising determining whether modified levels of a gene from Tables 10-12 and 14 is present in a cell, wherein the presence of protein is predictive of the patient's likelihood to respond to a drug treatment for the condition. Examples of the prevention or improvement of symptoms accompanied by psoriasis that can monitored for effectiveness include prevention or improvement of inflammation, hyperproliferation of the epidermis, altered maturation of the epidermis (resulting in scaling), vascular alterations (which add to redness) and loss of the granular layer.

The invention also provides a method of predicting a response to therapy in a subject having psoriasis by determining the presence or absence in the subject of one or more markers associated with psoriasis described in Tables 2-14, diagnosing the subject in which the one or more markers are present as having psoriasis, and predicting a response to a therapy based on the diagnosis e.g., response to therapy may include an efficacious response and/or one or more adverse events. The invention also provides a method of optimizing therapy in a subject having psoriasis by determining the presence or absence in the subject of one or more markers associated with a clinical subtype of psoriasis, diagnosing the subject in which the one or more markers are present as having a particular clinical subtype of psoriasis, and treating the subject having a particular clinical subtype of psoriasis based on the diagnosis. As an example, treatment for the guttate psoriasis currently includes presumptive antistreptococcal antibiotic therapy. Although widespread and usually explosive in onset, guttate psoriasis often rapidly responds to sunlight or ultraviolet light therapy. To give optimum treatment, the clinician must find out: which aspects and to what extent the disease worries the patient and what type of improvement would substantially reduce this worry, In addition to the cost and risk of treatment. Current pharmacological treatments are either modestly effective or have substantial risk. Psoriasis is a dynamic disease and treatment risks usually increase with cumulative doses of a specific therapy. Optimum treatment requires periodic re-evaluation often leading to changes in treatment. Because of its chronic nature, psoriasis is a great burden to many patients and a constant challenge to the clinician.

### Examples

### Example 1

### Identification of Cases and Controls

All individuals were sampled from the Quebec founder population (QFP). Membership in the founder population was defined as having four grandparents with French Canadian family names who were born in the Province of Quebec, Canada or in adjacent areas of the Provinces of New Brunswick and Ontario or in New England or New York State. The Quebec founder population has two distinct advantages over general populations for LD mapping. Because it is relatively young, about 12 to 15 generations from the mid 17th century to the present, and because it has a limited but sufficient number of founders, approximately 2600 effective founders (Charbonneau et al. 1987), the Quebec population is characterized both by extended LD and by decreased genetic heterogeneity. The increased extent of LD allows the detection of disease genes using a reasonable marker density, while still allowing the increased meiotic resolution of population-based mapping. The number of founders is small enough to result in increased LD and reduced allelic heterogeneity, yet large enough to insure that all of the major disease genes involved in general populations are present in Quebec. Reduced allelic heterogeneity will act to increase relative risk imparted by the remaining alleles and so increase the power of case/control studies to detect gene an associated allele, a particular allele of a polymorphic locus, or the likes involved in complex diseases within the Quebec population. The specific combination of age in generations, optimal number of founders and large present population size makes the QFP optimal for LD-based gene mapping.

Patient inclusion criteria for the study included diagnosis for plaque forming psoriasis vulgaris with the onset of the condition occurred between age 18 and 40.

Patients that were diagnosed with inverse, palmo-plantar, pustular, erythrodermic, guttate and isolated dermatological lesions were excluded from the study. All human sampling was subject to ethical review procedures.

All enrolled QFP subjects (patients and family members) provided a 30 ml blood sample (3 barcoded tubes of 10 ml). Samples were processed immediately upon arrival at Genizon's laboratory. All samples were scanned and logged into a LabVantage Laboratory Information Management System (LIMS), which served as a hub between the clinical data management system and the genetic analysis system. Following centrifugation, the buffy coat containing the white blood cells was isolated from each tube. Genomic DNA was extracted from the buffy coat from one of the tubes, and stored at 4° C. until required for genotyping. DNA extraction was performed with a commercial kit using a guanidine hydrochloride based method (FlexiGene, Qiagen) according to the manufacturer's instructions. The extraction method yielded high molecular weight DNA, and the quality of every DNA sample was verified by agarose gel electrophoresis. Genomic DNA appeared on the gel as a large band of very high molecular weight. The remaining two buffy coats were stored at −80° C. as backups.

The samples were collected as family trios from psoriasis subjects and two close relatives. All the 500 trios were Parent, Parent, Child (PPC) trios. Therefore, only PPC trios were used for the analysis reported here. One member of each trio was affected with psoriasis. When a child was the affected member of the trio, the two non-transmitted parental chromosomes (one from each parent) were used as controls, when one of the parents was affected, that person's spouse provided the control chromosomes. The recruitment of trios allowed the precise determination of haplotypes.

### Example 2

### Genome Wide Association

Genotyping was performed using Perlegen's ultra-high-throughput platform. Loci of interest were amplified and hybridized to wafers containing arrays of oligonucleotides. Allele discrimination was performed through allele-specific hybridization. In total, 80,654 SNPs, with a variable density adjusted to the extent of local LD, were genotyped on the 500 trios for a total of 97,994,610 genotypes. This set of markers constitutes the QLDM (Quebec LD Map), a map created specifically for the Quebec founder population. Briefly, it possesses a base density of one marker per 40 kb and up to one marker per 10 kb in low-LD regions, the lower the LD is in a given area, the higher the marker density will be. The markers were selected from various databases including the ˜1.6 million SNP database of Perlegen Life Sciences (Patil, 2001), the hapmap consortium database and dbSNP at NCBI. The SNPs were chosen to maximize uniformity of genetic coverage and as much as possible, with a minor allele frequency of 10% or higher. All SNPs that did not pass the quality controls for the assay, that had a high rate of Mendelian error (cut-off at 1% Mendelian error rate), that deviated from the Hardy-Weinberg equilibrium (calculated with control chromosomes only), that had too many missing data (cut-off at 5% missing values or higher), or that were not sufficiently polymorphic in the Quebec founder population (cut-off at 10% MAF or below) were removed from the analysis. Genetic analysis was performed on a total of 61,171 SNPs (58,461 autosomal, 2660 X chromosome 18 Y chromosome and 32 SNPs from the X-Y pseudoautosomal region).

The genotyping information was entered into a Unified Genotype Database (a proprietary database under development) from which it was accessed using custom-built programs for export to the genetic analysis pipeline via a custom-built system known as GeneSys. GeneSys is a proprietary system that was built to automate data analysis. It significantly speeds up the data analysis by automating most of the statistical genetics processes and serves as a warehouse for storage of the large amount of information collected. Analyses of these genotypes were performed with the statistical tools described in Example 3. The GWS permitted the identification of 80 candidate regions that are further analyzed by the Fine Mapping approach described below.

### Example 3

### Genetic Analysis

1. Dataset Quality Assessment

Prior to performing any analysis, the dataset from the GWS was verified for completeness of the trios. The program GGFileMod removed any trios with abnormal structure or missing individuals (e.g. trios without a proband, duos, singletons, etc.), and calculated the total number of complete trios in the dataset. The trios were also tested to make sure that no subjects within the cohort were related more closely than second cousins (6 meiotic steps).

Subsequently, the program DataCheck2.1 was used to calculate the following statistics per marker and per family:
Minor allele frequency (MAF) for each marker;
Missing values for each marker and family;
Hardy Weinberg Equilibrium for each marker; and
Mendelian segregation error rate.

The following acceptance criteria were applied for internal analysis purposes:

MAF>10%; Missing values<5%; Observed non-Mendelian segregation<1%; and Allele frequencies for controls in Hardy Weinberg Equilibrium.

Analyses of variance were performed using the algorithm GenAnova, to assess whether families or markers had the greater effect on missing values and non-Mendelian segregation. This was used to determine the smallest number of data points to remove from the dataset to meet the requirements for missing values and non-Mendelian segregation. Markers or families not meeting the criteria were removed from the dataset in the following step. The families and/or markers were removed from the dataset using the program DataPull, which generates an output file that is used for subsequent analysis of the genotype data.

2. Phase Determination

The Program PhaseFinderSNP2.0 was used to determine phase from trio data on a marker-by-marker, trio-by-trio basis. The output file contains haplotype data for all trio members, containing ambiguities where all trio members are heterozygous or where data is missing. The program FileWriterTemp was then used to determine case and control haplotypes and to prepare the data in the proper input format for the next stage of analysis, using the expectation maximization algorithm, PL-EM, to call phase on the remaining ambiguities. This stage consists of several modules for resolution of the remaining phase ambiguities. PLEMInOut1 was first used to recode the haplotypes for input into the PL-EM algorithm in 15-marker blocks. The haplotype information was encoded as genotypes, allowing for the entry of known phase into the algorithm, which limits the possible number of estimated haplotypes. The PL-EM algorithm was used to estimate haplotypes from the “genotype” data in 15-marker windows, advancing in increments of one marker across the chromosome. The results were then converted into multiple 15-marker haplotype files using the program PLEMInOut2. Subsequently PLEMBlockGroup was used to convert the individual 15-marker block files into one continuous block of haplotypes for the entire chromosome, and to generate files for further analysis by LDSTATS, Hapfreq and HapColor. PLEMBlockGroup takes the consensus estimation of the allele call at each marker over all separate estimations (most markers are estimated 15 different times as the 15 marker blocks pass over their position).

3. Haplotype Association Analysis

Haplotype association analysis was performed using the program LDSTATS. LDSTATS tests for association of haplotypes with the disease phenotype. The algorithms LDSTATS (v2.0) and LDSTATS (v4.0) define haplotypes using multi-marker windows that advance across the marker map in one-marker increments. Windows can contain any odd number of markers specified as a parameter of the algorithm. Other marker windows can also be used. At each position the frequency of haplotypes in cases and controls was calculated and a chi-square statistic was calculated from case control frequency tables. For LDSTATS v2.0, the significance of the chi-square for single marker and 3-marker windows was calculated as Pearson's chi-square with degrees of freedom. Larger windows of multi-allelic haplotype association were tested using Smith's normalization of the square root of Pearson's Chi-square. In addition, LDSTATS v2.0 calculates Chi-square values for the transmission disequilibrium test (TDT) for single markers in situations where the trios consisted of parents and an affected child.

LDSTATS v4.0 calculates significance of chi-square values using a permutation test in which case-control status is randomly permuted until 350 permuted chi-square values are observed that are greater than or equal to chi-square value of the actual data. The P value is then calculated as 350/the number of permutations required.

Table 2 lists the results for association analysis using LDSTATs (v2.0 and v4.0) for the candidate regions described above based on the genome wide scan genotype data. For each region that was associated with Psoriasis disease in the genome wide scan, we report in Table 3 the allele frequencies and the relative risk (RR) for the haplotypes contributing to the best signal at each SNP in the region. The best signal at a given location was determined by comparing the significance (p-value) of the association with Psoriasis disease for window sizes of 1, 3, 5, 7, and 9 SNPs, and selecting the most significant window. For a given window size at a given location, the association with Psoriasis disease was evaluated by comparing the overall distribution of haplotypes in the cases with the overall distribution of haplotypes in the controls. Haplotypes with a relative risk greater than one increase the risk of developing Psoriasis disease while haplotypes with a relative risk less than one are protective and decrease the risk.

5. Conditional Haplotype Analyses

Conditional haplotype analyses were performed on subsets of the original set of 500 cases and 500 controls using the program LDSTATS (v2.0). The selection of a subset of cases and their matched controls was based on the carrier status of cases at a gene or locus of interest. We selected seven loci based on our association findings using LDSTAT (v2.0) with 500 trios (see below). The first conditional analysis was performed using a locus in region 32 on chromosome 4. The most significant association was obtained in the gene PDGFC with a SNPs corresponding to SEQ IDs 33380. Allele T was the risk allele. We partitioned the cases into two groups; the first group consisting of those cases that were carrier of the risk allele and the second group consisting of the remaining cases, the non-carriers. The resulting sample sizes were respectively 271 and 206. LDSTAT (v2.0) was run in each group and regions showing association with Psoriasis are reported in Table 4. Ten regions (143, 272, 315, 374, 389, 484, 490, 534, 609, 691) were associated with Psoriasis in the group of carriers (has_{—}32_caserisk), indicating the presence of an epistatic interaction between risk factors in that region and risk factors in region 32 (Table 4). Ninety one regions (147, 149, 150, 161, 163, 165, 171, 172, 174, 177, 178, 180, 187, 188, 193, 199, 211, 214, 219, 220, 235, 236, 248, 257, 260, 265, 267, 277, 293, 301, 319, 325, 338, 347, 349, 351, 362, 370, 380, 396, 407, 418, 433, 444, 446, 449, 461, 468, 473, 478, 482, 497, 509, 512, 514, 530, 539, 548, 561, 566, 580, 595, 596, 599, 607, 608, 614, 621, 622, 623, 625, 628, 630, 631, 632, 634, 645, 649, 658, 660, 672, 673, 678, 679, 682, 683, 703, 716, 718, 724, 728) were associated with Psoriasis in the group of non-carriers (not_{—}32_caserisk), indicating the existence of risk factors acting independently of risk factors in region 32. We repeated the process of partitioning the cases into two groups using allele C, the protective allele. The resulting sample sizes were 397 and 80 for the group of carriers and non-carriers respectively. Thirty seven regions (152, 170, 179, 226, 229, 234, 245, 247, 249, 305, 321, 333, 340, 348, 378, 383, 424, 435, 438, 454, 455, 462, 475, 489, 529, 550, 569, 587, 598, 642, 650, 657, 667, 670, 681, 704, 715) were associated with Psoriasis in the group of carriers (has_{—}32_caseprotective), indicating the existence of risk factors acting independently of risk factors in region 32.

The second conditional analysis was performed using a locus in region 50 (PSORS1) on chromosome 6. The most significant association was obtained with a haplotype window of size 5 containing SNPs corresponding to SEQ IDs 31191, 31192, 31194, 31195 and 31199. A reduced haplotype diversity was observed and we selected a risk haplotype, CACCT, and a protective haplotype, TATCT, for conditional analyses. Using the risk haplotype, we partitioned the cases into two groups; the first group consisting of those cases that were carrier of the risk haplotype and the second group consisting of the remaining cases, the non-carriers. The resulting sample sizes were respectively 168 and 294. LDSTATS (v2.0) was run in each group and regions showing association with Psoriasis are reported in Table 4. Fourteen regions (146, 157, 312, 354, 355, 376, 406, 503, 504, 602, 603, 615, 685, 692) were associated with Psoriasis in the group of carriers (has_{—}50_caserisk), indicating the presence of an epistatic interaction between risk factors in those regions and risk factors in region 50 (Table 4). Two regions (278, 528) were associated with Psoriasis in the group of non-carriers (not_{—}50_caserisk), indicating the existence of risk factors acting independently of risk factors in region 50. Using the protective haplotype, we partitioned the cases into two groups; the first group consisting of those cases that were carrier of the protective haplotype and the second group consisting of the remaining cases, the non-carriers. The resulting sample sizes were respectively 110 and 352. LDSTATS (v2.0) was run in each group and regions showing association with Psoriasis are reported in Table 4. Three regions (289, 439, 451) were associated with Psoriasis in the group of carriers (has_{—}50_caseprotective), indicating the existence of risk factors acting independently of risk factors in region 50. Forty nine regions (153, 158, 197, 206, 212, 232, 255, 273, 275, 281, 287, 303, 309, 313, 322, 328, 335, 345, 352, 358, 382, 385, 397, 400, 405, 412, 417, 422, 431, 456, 480, 515, 521, 526, 541, 552, 559, 564, 568, 572, 577, 593, 624, 647, 664, 665, 688, 706, 711) were associated with Psoriasis in the group of non-carriers (not_{—}50_caseprotective), indicating the presence of an epistatic interaction between risk factors in those regions and risk factors in region 50 (Table 4). Due to the dominance effect of the risk haplotype, we also considered the protective haplotype while excluding heterozygotes involving the risk haplotype. The sample size for the carriers was 84 and for the non-carriers 378. LDSTATS (v2.0) was run in each group and regions showing association with Psoriasis are reported in Table 4. Twelve regions (176, 290, 291, 375, 441, 474, 553, 590, 597, 669, 684, 721) were associated with Psoriasis in the group of non-carriers (has_{—}50_caseprotective_not_risk), indicating the existence of risk factors acting independently of risk factors in region 50. One hundred and eighteen regions (142, 154, 159, 160, 162, 166, 173, 183, 191, 194, 195, 198, 203, 204, 207, 209, 213, 217, 222, 228, 231, 239, 242, 251, 253, 256, 261, 264, 268, 269, 274, 276, 282, 288, 304, 310, 316, 323, 324, 329, 336, 346, 353, 357, 359, 379, 384, 386, 387, 388, 391, 395, 398, 401, 402, 403, 404, 409, 413, 423, 432, 440, 450, 453, 457, 466, 471, 476, 479, 481, 483, 485, 492, 494, 496, 500, 510, 516, 519, 520, 527, 538, 542, 545, 560, 562, 563, 567, 573, 576, 585, 589, 592, 594, 604, 611, 613, 618, 619, 620, 626, 635, 648, 661, 662, 663, 666, 668, 675, 680, 689, 698, 702, 705, 707, 712, 720, 725) were associated with Psoriasis in the group of non-carriers (not_{—}50_caseprotective_not_risk), indicating the presence of an epistatic interaction between risk factors in these regions and risk factors in region 50 (Table 4).

The third conditional analysis was performed using a locus in region 78 on chromosome 10. The most significant association was obtained with a SNPs corresponding to SEQ IDs 34384. Allele G was the risk allele. We partitioned the cases into two groups; the first group consisting of those cases that were carrier of the risk allele and the second group consisting of the remaining cases, the non-carriers. The resulting sample sizes were respectively 284 and 192. LDSTAT (v2.0) was run in each group and regions showing association with Psoriasis are reported in Table 4. Nine regions (190, 252, 298, 445, 448, 467, 488, 629, 671) were associated with Psoriasis in the group of carriers (has_{—}78_caserisk) indicating the presence of an epistatic interaction between risk factors in those regions and risk factors in region 78 (Table 4). We repeated the process of partitioning the cases into two groups using allele C, the protective allele. The resulting sample sizes were 402 and 74 for the group of carriers and non-carriers respectively. One region (499) was associated with Psoriasis in the group of non-carriers (not_{—}78_caseprotective), indicating the presence of an epistatic interaction between risk factors in those regions and risk factors in region 78 (Table 4).

The fourth conditional analysis was performed using a locus in region 99 on chromosome 12. The most significant association was obtained in gene SCARB1 with a SNPs corresponding to SEQ IDs 34811. Allele A was the protective allele. We partitioned the cases into two groups; the first group consisting of those cases that were carrier of a protective haplotype and the second group consisting of the remaining cases, the non-carriers. The resulting sample sizes were respectively 195 and 278. LDSTAT (v2.0) was run in each group and regions showing association with Psoriasis are reported in Table 4. Eleven regions (306, 342, 373, 427, 487, 493, 544, 639, 651, 676, 690) were associated with Psoriasis in the group of carriers (has_{—}99_caseprotective), indicating the existence of risk factors acting independently of risk factors in region 99. Eleven regions (280, 294, 297, 308, 334, 360, 366, 540, 557, 686, 701) was associated with Psoriasis in the group of non-carriers (not_{—}99_protective), indicating the presence of an epistatic interaction between risk factors in that region and risk factors in region 99 (Table 4).

The fifth conditional analysis was performed using a locus in region 110 on chromosome 15. The most significant association was obtained in gene GABRG3 with a haplotype window of size 7 containing SNPs corresponding to SEQ IDs 35144, 35145, 32221, 35146, 35148, 35149 and 35150. A reduced haplotype diversity was observed and we selected a set of risk haplotypes for conditional analyses. The set consisted of haplotypes GCATCCG, ACATGTA and the haplotype GTGTCCG but only in homozygote form, GTGTCCG/GTGTCCG. We partitioned the cases into two groups; the first group consisting of those cases that were carrier of a risk haplotype and the second group consisting of the remaining cases, the non-carriers. The resulting sample sizes were respectively 129 and 353. LDSTAT (v2.0) was run in each group and regions showing association with Psoriasis are reported in Table 4. Twenty seven regions (144, 151, 164, 182, 205, 218, 221, 223, 230, 244, 258, 286, 369, 470, 502, 505, 518, 546, 549, 565, 574, 575, 578, 633, 656, 677, 708) were associated with Psoriasis in the group of carriers (has_{—}110_caserisk), indicating the presence of an epistatic interaction between risk factors in that region and risk factors in region 110 (Table 4). Twenty two regions (155, 175, 202, 227, 292, 295, 356, 410, 425, 465, 495, 506, 508, 532, 547, 554, 586, 601, 641, 643, 694, 709) was associated with Psoriasis in the group of non-carriers (not_{—}110_caserisk), indicating the existence of risk factors acting independently of risk factors in region 110.

The sixth conditional analysis was performed using a locus in region 125 on chromosome 20. The most significant association was obtained in gene TOP1 with a haplotype window of size 5 containing SNPs corresponding to SEQ IDs 32488, 35437, 35438, 35439 and 32490 (see Table below for conversion to the specific DNA alleles used). A reduced haplotype diversity was observed and we selected one risk and one protective haplotype for conditional analyses. The risk haplotype was CATTC and the protective haplotype was CGCCG. Using the risk haplotype, we partitioned the cases into two groups; the first group consisting of those cases that were carrier of the risk haplotype and the second group consisting of the remaining cases, the non-carriers. The resulting sample sizes were respectively 375 and 95. LDSTATS (v2.0) was run in each group and regions showing association with Psoriasis are reported in Table 4. Eleven regions (283, 332, 343, 365, 414, 428, 523, 535, 583, 591, 652) were associated with Psoriasis in the group of carriers (has_{—}125_caserisk) indicating the presence of an epistatic interaction between risk factors in those regions and risk factors in region 125 (Table 4). Thirty two regions (148, 167, 186, 200, 210, 215, 224, 250, 262, 263, 270, 296, 317, 318, 320, 337, 377, 399, 411, 419, 421, 436, 459, 531, 543, 581, 606, 627, 636, 659, 693, 713) were associated with Psoriasis in the group of non-carriers (not_{—}125_caserisk), indicating the existence of risk factors acting independently of risk factors in region 125. Because the protective haplotype appears to be dominant, we also considered separately individuals that were homozygote for the risk haplotype. We partitioned the cases into two groups. The resulting sample sizes were 166 and 304 for the group of homozygote carriers and for the remainder, respectively. Three regions (331, 371, 524) were associated with Psoriasis in the group of carriers (has_{—}125_caserisk_homozygote) indicating the presence of an epistatic interaction between risk factors in those regions and risk factors in region 125 (Table 4). Additionally, using the risk haplotype while excluding heterozygotes with the protective haplotype, we partitioned the cases into two groups. The resulting sample sizes were 243 and 227 for the group of carriers and non-carriers respectively. Twenty two regions (192, 238, 284, 307, 314, 330, 372, 393, 429, 477, 486, 491, 525, 536, 582, 612, 654, 674, 700, 717, 726, 727) were associated with Psoriasis in the group of carriers (has_{—}125_caserisk_not_protective), indicating the presence of an epistatic interaction between risk factors in those regions and risk factors in region 125 (Table 4). Three regions (184, 350, 517) were associated with Psoriasis in the group of non-carriers (not_{—}125_caserisk_not_protective), indicating the existence of risk factors acting independently of risk factors in region 125. We repeated the process of partitioning the cases into two groups using the protective haplotype. Due to the dominance effect of the risk haplotype, the first group consisted of those cases that were carrier of the protective haplotype but not the risk haplotype and the second group consisting of the remaining cases, the non-carriers. The resulting sample sizes were 72 and 398 for the group of carriers and non-carriers respectively. Eight regions (168, 185, 243, 420, 498, 501, 579, 637) were associated with Psoriasis in the group of non carriers (has_{—}125_caseprotective_not_risk) indicating the existence of risk factors acting independently of risk factors in region 125 (Table 4). Twenty regions (254, 285, 302, 311, 327, 344, 361, 367, 394, 415, 430, 537, 558, 571, 584, 610, 616, 653, 655, 697) were associated with Psoriasis in the group of non-carriers (not_{—}125_caseprotective_not_risk) indicating the presence of an epistatic interaction between risk factors in those regions and risk factors in region 125 (Table 4).

A seventh conditional analysis was performed using a locus in region 131 on chromosome 22. The most significant association was obtained in the gene IL2RB with a SNPs corresponding to SEQ IDs 32576. Allele G was the risk allele. We partitioned the cases into two groups; the first group consisting of those cases that were carrier of the risk allele and the second group consisting of the remaining cases, the non-carriers. The resulting sample sizes were respectively 442 and 23. LDSTATS (v2.0) was run in each group and regions showing association with Psoriasis are reported in Table 4. Twelve regions (196, 279, 326, 364, 392, 426, 522, 533, 556, 570, 687, 696) were associated with Psoriasis in the group of carriers (has_{—}131_caserisk), indicating the presence of an epistatic interaction between risk factors in those regions and risk factors in region 131 (Table 4). Two regions (408, 617) were associated with Psoriasis in the group of non-carriers (not_{—}131_caserisk), indicating the presence of risk factors acting independently of risk factors in region 131. We repeated the process of partitioning the cases into two groups using allele A, the protective allele. The resulting sample sizes were 192 and 273 for the group of carriers and non-carriers respectively. One region (363) was associated with Psoriasis in the group of carriers (has_{—}131_caseprotective), indicating the presence of risk factors acting independently of risk factors in region 131. Fifty seven regions (141, 145, 156, 169, 181, 189, 201, 208, 216, 225, 233, 237, 240, 241, 246, 259, 266, 271, 299, 300, 339, 341, 368, 381, 390, 416, 434, 437, 442, 443, 447, 452, 458, 460, 463, 464, 469, 472, 507, 511, 513, 551, 555, 588, 600, 605, 638, 640, 644, 646, 695, 699, 710, 714, 719, 722, 723) were associated with Psoriasis in the group of non-carriers (not_{—}131_caseprotective), indicating the presence of an epistatic interaction between risk factors in these regions and risk factors in region 131 (Table 4).

For each region that was associated with Psoriasis in the conditional analyses, we report in Table 5 the allele frequencies and the relative risk (RR) for the haplotypes contributing to the best signal centered at each SNP in the region. The best signal at a given location was determined by comparing the significance (p-value) of the association with Psoriasis for window sizes of 1, 3, 5, 7, and 9 SNPs, and selecting the most significant window. For a given window size at a given location, the association with Psoriasis was evaluated by comparing the overall distribution of haplotypes in the cases with the overall distribution of haplotypes in the controls. Haplotypes with a relative risk greater than one increase the risk of developing Psoriasis while haplotypes with a relative risk less than one are protective and decrease the risk.

In Table 8, we report the allele frequencies and the relative risk (RR) for the haplotypes contributing to the best signal centered at each SNP in the fine mapping regions for every conditional analysis described above, and Table 9 reports the allele frequencies and the relative risk (RR) for the haplotypes contributing to the best signal centered at each SNP in the regions reported in Table 8.

DNA alleles used in haplotypes (Region 32)

| SeqID | 33380 |
|---|---|
| Risk | |
| 1 | T |
| Protective | |
| 2 | C |

DNA alleles used in haplotypes (Region 50)

| SeqID | 31191 | 31192 | 31194 | 31195 | 31199 |
|---|---|---|---|---|---|
| Risk | | | | | |
| 12112 | C | A | C | C | T |
| Protective | | | | | |
| 22212 | T | A | T | C | T |

DNA alleles used in haplotypes (Region 78)

| SeqID | 34384 |
|---|---|
| Risk | |
| 2 | G |
| Protective | |
| 1 | C |

DNA alleles used in haplotypes (Region 99)

| SeqID | 34811 |
|---|---|
| Protective | |
| 1 | A |

DNA alleles used in haplotypes (Region 110)

| SeqID | 35144 | 35145 | 32221 | 35146 | 35148 | 35149 | 35150 |
|---|---|---|---|---|---|---|---|
| Risk | | | | | | | |
| 2221122 | G | C | A | T | C | C | G |
| 1221211 | A | C | A | T | G | T | A |
| Risk/ | | | | | | | |
| Homozygote | | | | | | | |
| form | | | | | | | |
| 2111122/ | G/G | T/T | G/G | T/T | C/C | C/C | G/G |
| 2111122 | | | | | | | |

DNA alleles used in haplotypes (Region 125)

| SeqID | 32488 | 35437 | 35438 | 35439 | 32490 |
|---|---|---|---|---|---|
| Risk | | | | | |
| 11111 | C | A | T | T | C |
| Protective | | | | | |
| 12222 | C | G | C | C | G |

DNA alleles used in haplotypes (Region 131)

| SeqID | 32576 |
|---|---|
| Risk | |
| 1 | G |
| Protective | |
| 2 | A |

5. Singletype Analysis

The SINGLETYPE algorithm assesses the significance of case-control association for single markers using the genotype data from the laboratory as input in contrast to LDSTATS single marker window analyses, in which case-control alleles for single markers from estimated haplotypes in file, hapatctr.txt, as input. SINGLETYPE calculates P values for association for both alleles, 1 and 2, as well as for genotypes, 11, 12, and 22, and plots these as −log₁₀ P values for significance of association against marker position.

### Example 4

### Fine Mapping

The top regions identified as being associated with psoriasis by the GWS are further analyzed by fine mapping using a denser set of markers, in order to validate and/or refine the signal. The fine mapping is carried out using the Illumina BeadStation 500GX SNP genotyping platform. Alleles are genotyped using an allele-specific elongation assay that involves ligation to a locus-specific oligonucleotide. The assay is performed directly on genomic DNA at a highly multiplex level and the products are amplified using universal oligonucleotides. For each candidate region, a set of SNP markers was selected with an average inter-marker distance of 1-4 Kb distributed over about 400 Kb to 1 Mb and were roughly centered at the highest point of the GWS curves. The cohort used for the fine mapping consisted of 500 Psoriasis disease trios (as for the GWS). The algorithms used for genetic analyses were the same as used in the GWS and are described in Example 3. Table 6 lists the fine mapping SNPs for the confirmed regions and their respective p values using 500 trios and two analysis methods: LDSTATS (v2.0) and LDSTATS (v4.0). For each region that was associated with Psoriasis disease in the fine mapping analyses, we report in Table 7 the allele frequencies and the relative risk (RR) for the haplotypes contributing to the best signal at each SNP in the region. The best signal at a given location was determined by comparing the significance (p-value) of the association with Psoriasis disease for multiple window sizes, and selecting the most significant window. For a given window size at a given location, the association with Psoriasis disease was evaluated by comparing the overall distribution of haplotypes in the cases with the overall distribution of haplotypes in the controls. Haplotypes with a relative risk greater than one increase the risk of developing Psoriasis disease while haplotypes with a relative risk less than one are protective and decrease the risk.

### Example 5

### Gene Identification and Characterization

A series of gene characterization was performed for each candidate region described in Table 1. Any gene or EST mapping to the interval based on public map data or proprietary map data was considered as a candidate Psoriasis disease gene. The approach used to identify all genes located in the critical regions is described below.

Public Gene Mining

Once regions were identified using the analyses described above, a series of public data mining efforts were undertaken, with the aim of identifying all genes located within the critical intervals as well as their respective structural elements (i.e., promoters and other regulatory elements, UTRs, exons and splice sites). The initial analysis relied on annotation information stored in public databases (e.g. NCBI, UCSC Genome Bioinformatics, Entrez Human Genome Browser, OMIM—see below for database URL information). Tables 10-12 and 14 lists the genes that have been mapped to the candidate regions.

Database URLs

| Name | URL |
|---|---|
| Biocarta | http://www.biocarta.com/ |
| BioCyc | http://www.biocyc.org/ |
| Bimolecular Interaction Network | http://bind.ca/ |
| Database (BIND) | |
| Database of Interacting Proteins | http://dip.doe-mbi.ucla.edu/ |
| ECgene EST clustering | http://genome.ewha.ac.kr/ECgene/, also available at |
| | http://genome.ucsc.edu/index.html?org=Human |
| Gene Expression Omnibus | http://www.ncbi.nlm.nih.gov/geo/ |
| Human Genome Browser | http://www.ensembl.org/Homo_sapiens/ |
| Interdom | http://interdom.lit.org.sg/help/term.php |
| Kyoto Encyclopedia of Genes and | http://www.genome.jp/kegg/ |
| Genomes (KEGG) | |
| Molecular Interactions Database | http://mint.bio.uniroma2.it/mint/ |
| (MINT) | |
| National Center for Biotechnology | http://www.ncbi.nlm.nih.gov/ |
| Information (NCBI) | |
| Online Mendelian Inheritance in | http://www.ncbi.nlm.nih.gov/entrez/query.fcgi?db=OMIM |
| Man (OMIM) | |
| OmniViz | http://www.omniviz.com/applications/omni_viz.htm |
| Pathway Enterprise | http://www.omniviz.com/applications/pathways.htm |
| Reactome | http://www.reactome.org/ |
| Transpath | http://www.biobase.de/pages/products/transpath.html |
| UCSC Genome Bioinformatics | http://genome.ucsc.edu/index.html?org=Human |
| UniGene | http://www.ncbi.nlm.nih.gov/entrez/query.fcgi?db=unigene |

For some genes the available public annotation was extensive, whereas for others very little was known about a gene's function. A more detailed analysis was therefore performed to characterize genes that corresponded to this latter class. Importantly, the presence of rare splice variants and artifactual ESTs was carefully evaluated. The public data derived from the EST clustering software ECgene (Kim et al., 2005) were also examined for additional gene models. This tool combines genome-based EST clustering and transcript assembly to generate a group of alternatively spliced consensi. This analysis of novel ESTs provided an indication of additional gene content in some regions. The resulting clusters were graphically displayed against the genomic sequence, providing indications of separate clusters that may contribute to the same gene, thereby facilitating development of confirmatory experiments in the laboratory.

A unique consensus sequence was constructed for each splice variant and a trained reviewer assessed each alignment. This assessment included examination of all putative splice junctions for consensus splice donor/acceptor sequences, putative start codons, consensus Kozak sequences and upstream in-frame stops, and the location of polyadenylation signals. In addition, conserved noncoding sequences (CNSs) that could potentially be involved in regulatory functions were included as important information for each gene. The genomic reference and exon sequences were then archived for future reference. A master assembly that included all splice variants, exons and the genomic structure was used in subsequent analyses (i.e., analysis of polymorphisms).

An important component of these efforts was the ability to visualize and store the results of the data mining efforts. A customized version of the highly versatile genome browser GBrowse (http://www.gmod.org/) was implemented in order to permit the visualization of several types of information against the corresponding genomic sequence. In addition, the results of the statistical analyses were plotted against the genomic interval, thereby greatly facilitating focused analysis of gene content.

2. Computational Analysis of Genes and GeneMaps

In order to assist in the prioritization of candidate genes for which minimal annotation existed, a series of computational analyses were performed that included basic BLAST searches and alignments to identify related genes. In some cases this provided an indication of potential function. In addition, protein domains and motifs were identified that further assisted in the understanding of potential function, as well as predicted cellular localization.

A comprehensive review of the public literature was also performed in order to facilitate identification of information regarding the potential role of candidate genes in the pathophysiology of psoriasis. In addition to the standard review of the literature, public resources (Medline and other online databases) were also mined for information regarding the involvement of candidate genes in specific signaling pathways. A variety of pathway and yeast two hybrid databases were mined for information regarding protein-protein interactions.

These included BIND, MINT, DIP, Interdom, and Reactome, among others. By identifying homologues of genes in the psoriasis candidate regions and exploring whether interacting proteins had been identified already, knowledge regarding the GeneMaps for psoriasis was advanced. The pathway information gained from the use of these resources was also integrated with the literature review efforts, as described above.

3. Expression Studies

In order to determine the expression patterns for genes, relevant information was first extracted from public databases. The UniGene database, for example, contains information regarding the tissue source for ESTs and cDNAs contributing to individual clusters. This information was extracted and summarized to provide an indication in which tissues the gene was expressed. Particular emphasis was placed on annotating the tissue source for bona fide ESTs, since many ESTs mapped to Unigene clusters are artifactual. In addition, SAGE and microarray data, also curated at NCBI (Gene Expression Omnibus), provided information on expression profiles for individual genes. Particular emphasis was placed on identifying genes that were expressed in tissues known to be involved in the pathophysiology of psoriasis disorder.

Total human RNA samples from 24 different tissues as well as one Quantitative PCR (QPCR) Reference Total RNA sample were purchased from commercial sources (Clontech, Stratagene) and used as templates for first-strand cDNA synthesis with the High-Capacity cDNA Archive kit (Applied Biosystems) according to the manufacturer's instructions. A standard PCR protocol was used to amplify genes of interest from the original sample (50 ng cDNA); three serial dilutions of the cDNA samples corresponding to 5, 0.5 and 0.05 ng of cDNA were also tested. PCR products were separated by electrophoresis on a 96-well agarose gel containing ethidium bromide followed by UV imaging. The serial dilutions of the cDNA provided semi-quantitative determination of relative mRNA abundance. Tissue expression profiles were analyzed using standard gel imaging software (AlphaImager 2200); mRNA abundance was interpreted according to the presence of a PCR product in one or more of the cDNA sample dilutions used for amplification. For example, a PCR product present in all the cDNA dilutions (i.e. from 50 to 0.05 ng cDNA) was designated ++++ while a PCR product only detectable in the original undiluted cDNA sample (i.e., 50 ng cDNA) was designated as + (see Table 14). For each target gene, one or more gene-specific primer pairs were designed to span at least one intron when possible. Multiple primer-pairs targeting the same gene allowed comparison of the tissue expression profiles and controlled for cases of poor amplification.

4. Polymorphism Analysis

Polymorphisms identified in candidate genes, including those from the public domain as well as those identified by sequencing candidate genes and regions, are evaluated for potential function. Initially, polymorphisms are examined for potential impact upon encoded proteins. If the protein is a member of a gene family with reported 3-dimensional structural information, this information is used to predict the location of the polymorphism with respect to protein structure. This information provided insight into the potential role of polymorphisms in altering protein or ligand interactions, as well as suitability as a drug target. In a second phase of analysis we evaluate the potential role of polymorphisms in other biological phenomena, including regulation of transcription, splicing and mRNA stability, etc. There are many examples of the functional involvement of naturally occurring polymorphisms in these processes. As part of this analysis, polymorphisms located in promoter or other regulatory elements, canonical splice sites, exonic and intronic splice enhancers and repressors, conserved noncoding sequences and UTRs are localized.

### Example 6

### SNP and Polymorphisms Discovery (SNPD)

Candidate genes and regions were selected for sequencing in order to identify all polymorphisms. In cases where the critical interval, identified by fine mapping, was relatively small (˜50 kb), the entire region, including all introns, was sequenced to identify polymorphisms. In situations where the region is large (>50 kb), candidate genes were prioritized for sequencing, and/or only functional gene elements (promoters, exons and splice sites) were sequenced (see Table 13 for the SNPD of the invention).

The samples to be sequenced were selected according to which haplotypes contribute to the association signal observed in the region. The purpose is to select a set of samples that covered all the major haplotypes in the given region. Each major haplotype must be present in a few copies. The first step therefore consisted of determining the major haplotypes in the region to be sequenced.

Once a region was defined with the two boundary markers, all the markers used in fine mapping that are located within the region were used to determine the major haplotypes. Long haplotypes covering the whole region were thus inferred using the middle marker as an anchor. The results included two series of haplotype themes that define the major haplotypes, comparing the cases and the controls. This exercise was repeated using an anchor in the peripheral regions to ensure that major haplotype subsets that were not anchored at the original middle marker were not missed.

Once the major haplotypes were determined as described above, appropriate genomic DNA samples are selected such that each major haplotype and haplotype subset were represented in at least two to four copies.

The protocol includes the following steps, once a region is delimited:

1. Primer Design

The design of the primers is performed using a proprietary primer design tool. A primer quality control is included in the primer design process. Primers that successfully passed the control quality process were synthesized by Integrated DNA Technologies (IDT). The sense and anti-sense oligos are separated such that the sense oligos are placed on one plate in the same position as their anti-sense counterparts are on another plate. Two additional plates are created from each storage plate, one for use in PCR and the other for sequencing. For PCR, the sense and anti-sense oligos of the same pair are combined in the same well to achieve a final concentration of 1.5 μM for each oligonucleotide.

2. PCR Optimization

PCR conditions are optimized by testing a variety of conditions that included varying salt concentrations and temperatures, as well as including various additives. PCR products are checked for robust amplification and minimal background by agarose gel electrophoresis.

3. PCR on Selected Samples

PCR products to be used for sequencing are amplified using the conditions chosen during optimisation. The PCR products are purified free of salts, dNTPs and unincorporated primers by use of a MultiScreen PCR384 filter plate manufactured by Millipore. Following PCR, the amplicons are quantified by use of a lambda/Hind III standard curve. This is done to ensure that the quantity of PCR product required for sequencing had been generated. The raw data was measured against the standard curve data in Excel by use of a macro.

4. Sequencing

Sequencing of PCR products is performed by DNA Landmarks using ABI 3730 capillary sequencing instruments.

5. Sequence Analysis

The ABI Prism SeqScape software (Applied Biosystems) is used for SNP identification. The chromatogram trace files were imported into a SeqScape sequencing project and the base calling is automatically performed. Sequences are then aligned and compared to each other using the SeqScape program. The base calling is checked manually, base by base; editing was performed if needed.

### Example 7

### Ultra Fine Mapping (UFM)

Once polymorphisms are identified by sequencing efforts as described in Example 6, additional genotyping of all newly found polymorphisms is performed on the 500 PPC trios used in the fine mapping studies. Various types of genotyping assays may need to be utilized based on the type of polymorphism identified (i.e., SNP, indel, microsatellite). The assay type can be, but is not restricted to, Sentrix Assay Matrix on Illumina BeadStations, microsatellite on MegaBACE, SNP on ABI or Orchid. The frequencies of genotypes and haplotypes in cases and controls are analyzed in a similar manner as the GWS and fine mapping data. By examining all SNPs in a region, polymorphisms are identified that increase an individual's susceptibility to psoriasis. The goal of ultra-fine mapping is to identify the polymorphism that is most associated with disease phenotype as part of the search for the actual DNA polymorphism that confers susceptibility to disease. This statistical identification may need to be corroborated by functional studies.

### Example 8

### Confirmation of Candidate Regions and Genes in a General Population

The confirmation of any putative associations described in Example 7 is performed in an independent general population patient sample. These DNA samples consist of at least 400 trios or 750 patients with psoriasis and at least 750 controls.

All publications, patents and patent applications mentioned in the specification and reference list are herein incorporated by reference in their entirety for all purposes. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular CloningA LaboratoryManual, 2nd Ed., ed. by Sambrook, Fritsch and Maniatis (Cold Spring Harbor Laboratory Press: 1989); DNA Cloning, Volumes I and H (D. N. Glover ed., 4); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Haines & S. J. Higgins eds. 1984); Culture OfAnimal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### REFERENCES

1 Abbas, A. K., Lichtman, A. H. and Pober, J. S. (1994). Cellular and Molecular Immunology. Philadelphia, W B Saunders.
2 Abe, T., Oue, N., Yasui, W. and Ryoji, M. (2003). “Rapid and Preferential Induction of Atf3 Transcription in Response to Low Doses of Uva Light.” Biochem Biophys Res Commun 310(4): 1168-74.
3 Ackermann, L., Harvima, I. T., Pelkonen, J., Ritamaki-Salo, V., Naukkarinen, A., Harvima, R. J. and Horsmanheimo, M. (1999). “Mast Cells in Psoriatic Skin Are Strongly Positive for Interferon-Gamma.” Br J Dermatol 140(4): 624-33.
4 Allegra, M., Gagnoux-Palacios, L., Gache, Y., Rogues, S., Lestringant, G., Ortonne, J. P. and Meneguzzi, G. (2003). “Rapid Decay of Alpha6 Integrin Caused by a Mis-Sense Mutation in the Propeller Domain Results in Severe Junctional Epidermolysis Bullosa with Pyloric Atresia.” J Invest Dermatol 121(6): 1336-43.
5 Alwine, J. C., Kemp, D. J. and Stark, G. R. (1977). “Method for Detection of Specific Rnas in Agarose Gels by Transfer to Diazobenzyloxymethyl-Paper and Hybridization with DNA Probes.” Proc Natl Acad Sci USA 74(12): 5350-4.
6 Alwine, J. C., Reed, S. I. and Stark, G. R. (1977). “Characterization of the Autoregulation of Simian Virus 40 Gene A.” J Virol 24(1): 22-7.
7 Anderson, K. E., Coadwell, J., Stephens, L. R. and Hawkins, P. T. (1998). “Translocation of Pdk-1 to the Plasma Membrane Is Important in Allowing Pdk-1 to Activate Protein Kinase B.” Curr Biol 8(12): 684-91.
8 Anderson, W. F. (1992). “Human Gene Therapy.” Science 256(5058): 808-13.
9 Anton, I. M., de la Fuente, M. A., Sims, T. N., Freeman, S., Ramesh, N., Hartwig, J. H., Dustin, M. L. and Geha, R. S. (2002). “Wip Deficiency Reveals a Differential Role for Wip and the Actin Cytoskeleton in T and B Cell Activation.” Immunity 16(2): 193-204.
10 Athar, M., Kim, A. L., Ahmad, N., Mukhtar, H., Gautier, J. and Bickers, D. R. (2000). “Mechanism of Ultraviolet B-Induced Cell Cycle Arrest in G2/M Phase in Immortalized Skin Keratinocytes with Defective P53.” Biochem Biophys Res Commun 277(1): 107-11.
11 Austen, B. M. and Westwood, O. M. R. (1991). Protein Targeting and Secretion. New York, Oxford University Press.
12 Barany, F. (1991). “Genetic Disease Detection and DNA Amplification Using Cloned Thermostable Ligase.” Proc Natl Acad Sci USA 88(1): 189-93.
13 Barre, F. X., Ait-Si-Ali, S., Giovannangeli, C., Luis, R., Robin, P., Pritchard, L. L., Helene, C. and Harel-Bellan, A. (2000). “Unambiguous Demonstration of Triple-Helix-Directed Gene Modification.” Proc Natl Acad Sci USA 97(7): 3084-8.
14 Bartholin, L., Maguer-Satta, V., Hayette, S., Martel, S., Gadoux, M., Corbo, L., Magaud, J. P. and Rimokh, R. (2002). “Transcription Activation of Flrg and Follistatin by Activin a, through Smad Proteins, Participates in a Negative Feedback Loop to Modulate Activin a Function.” Oncogene 21(14): 2227-35.
15 Bishop, M. J., Ed. (1998). Guide to Human Genome Computing. Toronto, Academic Press.
16 Bivona, T. G., Perez De Castro, I., Ahearn, I. M., Grana, T. M., Chiu, V. K., Lockyer, P. J., Cullen, P. J., Pellicer, A., Cox, A. D. and Philips, M. R. (2003). “Phospholipase Cgamma Activates Ras on the Golgi Apparatus by Means of Rasgrp1.” Nature 424(6949): 694-8. Epub 2003 Jun. 29.
17 Bivona, T. G. and Philips, M. R. (2003). “Ras Pathway Signaling on Endomembranes.” Curr Opin Cell Biol 15(2): 136-42.
18 Bjerke, J. R., Tigalonova, M., Jensen, T. S. and Matre, R. (1994). “Fc Gamma-Receptors in Skin and Serum from Patients with Psoriasis, before and after Therapy.” Acta Derm Venereol Suppl 186: 141-2.
19 Blobe, G. C., Schiemann, W. P. and Lodish, H. F. (2000). “Role of Transforming Growth Factor Beta in Human Disease.” N Engl J Med 342(18): 1350-8.
20 Bourgeois, S. and Labuda, D. (2004). “Dynamic Allele-Specific Oligonucleotide Hybridization on Solid Support.” Anal Biochem 324(2): 309-11.
21 Bourguignon, L. Y., Singleton, P. A. and Diedrich, F. (2004). “Hyaluronan-Cd44 Interaction with Rac1-Dependent Protein Kinase N-Gamma Promotes Phospholipase Cgamma1 Activation, Ca(2+) Signaling, and Cortactin-Cytoskeleton Function Leading to Keratinocyte Adhesion and Differentiation.” J Biol Chem 279(28): 29654-69.
22 Bower, K. E., Zeller, R. W., Wachsman, W., Martinez, T. and McGuire, K. L. (2002). “Correlation of Transcriptional Repression by P21(Snft) with Changes in DNA.Nf-at Complex Interactions.” J Biol Chem 277(38): 34967-77. Epub 2002 Jun. 26.
23 Boyle, J. M., Grzeschik, K. H., Heath, P. R., Morten, J. E. and Stern, P. L. (1990). “Trophoblast Glycoprotein Recognised by Monoclonal Antibody 5t4 Maps to Human Chromosome 6q14-Q15.” Hum Genet 84(5): 455-8.
24 Browning, J. L., Dougas, I., Ngam-ek, A., Bourdon, P. R., Ehrenfels, B. N., Miatkowski, K., Zafari, M., Yampaglia, A. M., Lawton, P., Meier, W. and et al. (1995). “Characterization of Surface Lymphotoxin Forms. Use of Specific Monoclonal Antibodies and Soluble Receptors.” J Immunol 154(1): 33-46.
25 Cale, J. M., Shaw, C. E. and Bird, I. M. (1998). “Optimization of a Reverse Transcription-Polymerase Chain Reaction (Rt-Pcr) Mass Assay for Low-Abundance Mrna.” Methods Mol Biol 105: 351-71.
26 Campbell, J. S., Hughes, S. D., Gilbertson, D. G., Palmer, T. E., Holdren, M. S., Haran, A. C., Odell, M. M., Bauer, R. L., Ren, H. P., Haugen, H. S., Yeh, M. M. and Fausto, N. (2005). “Platelet-Derived Growth Factor C Induces Liver Fibrosis, Steatosis, and Hepatocellular Carcinoma.” Proc Natl Acad Sci USA 102(9): 3389-94. Epub 2005 Feb. 22.
27 Canessa, M. (1994). “Erythrocyte Sodium-Lithium Countertransport: Another Link between Essential Hypertension and Diabetes.” Curr Opin Nephrol Hypertens 3(5): 511-7.
28 Capon, F., Allen, M. H., Ameen, M., Burden, A. D., Tillman, D., Barker, J. N. and Trembath, R. C. (2004). “A Synonymous Snp of the Corneodesmosin Gene Leads to Increased Mrna Stability and Demonstrates Association with Psoriasis across Diverse Ethnic Groups.” Hum Mol Genet 13(20): 2361-8. Epub 2004 Aug. 27.
29 Caponi, L., Petit-Teixeira, E., Sebbag, M., Bongiorni, F., Moscato, S., Pratesi, F., Pierlot, C., Osorio, J., Chapuy-Regaud, S., Guerrin, M., Cornelis, F., Serre, G. and Migliorini, P. (2005). “A Family Based Study Shows No Association between Rheumatoid Arthritis and the Padi4 Gene in a White French Population.” Ann Rheum Dis 64(4): 587-93. Epub 2004 Oct. 14.
30 Cataisson, C., Joseloff, E., Murillas, R., Wang, A., Atwell, C., Torgerson, S., Gerdes, M., Subleski, J., Gao, J. L., Murphy, P. M., Wiltrout, R. H., Vinson, C. and Yuspa, S. H. (2003). “Activation of Cutaneous Protein Kinase C Alpha Induces Keratinocyte Apoptosis and Intraepidermal Inflammation by Independent Signaling Pathways.” J Immunol 171(5): 2703-13.
31 Chen, X., Levine, L. and Kwok, P. Y. (1999). “Fluorescence Polarization in Homogeneous Nucleic Acid Analysis.” Genome Res 9(5): 492-8.
32 Chien, C. T., Bartel, P. L., Sternglanz, R. and Fields, S. (1991). “The Two-Hybrid System: A Method to Identify and Clone Genes for Proteins That Interact with a Protein of Interest.” Proc Natl Acad Sci USA 88(21): 9578-82.
33 Clough, R. L., Sud, R., Davis-Silberman, N., Hertzano, R., Avraham, K. B., Holley, M. and Dawson, S. J. (2004). “Brn-3c (Pou4f3) Regulates Bdnf and Nt-3 Promoter Activity.” Biochem Biophys Res Commun 324(1): 372-81.
34 Cohen, A. S., Smisek, D. L. and Wang, B. H. (1996). “Emerging Technologies for Sequencing Antisense Oligonucleotides: Capillary Electrophoresis and Mass Spectrometry.” Adv Chromatogr 36: 127-62.
35 Cotton, R. G., Rodrigues, N. R. and Campbell, R. D. (1988). “Reactivity of Cytosine and Thymine in Single-Base-Pair Mismatches with Hydroxylamine and Osmium Tetroxide and Its Application to the Study of Mutations.” Proc Natl Acad Sci USA 85(12): 4397-401.
36 Cowell, I. G. and Austin, C. A., Eds. (1997). Cdna Library Protocols. Methods in Molecular Biology. Totowa, N.J., Humana Press.
37 Cronin, M. T., Fucini, R. V., Kim, S. M., Masino, R. S., Wespi, R. M. and Miyada, C. G. (1996). “Cystic Fibrosis Mutation Detection by Hybridization to Light-Generated DNA Probe Arrays.” Hum Mutat 7(3): 244-55.
38 Dalbeth, N., Gundle, R., Davies, R. J., Lee, Y. C., McMichael, A. J. and Callan, M. F. (2004). “Cd56bright Nk Cells Are Enriched at Inflammatory Sites and Can Engage with Monocytes in a Reciprocal Program of Activation.” J Immunol 173(10): 6418-26.
39 Dean, F. B., Hosono, S., Fang, L., Wu, X., Faruqi, A. F., Bray-Ward, P., Sun, Z., Zong, Q., Du, Y., Du, J., Driscoll, M., Song, W., Kingsmore, S. F., Egholm, M. and Lasken, R. S. (2002). “Comprehensive Human Genome Amplification Using Multiple Displacement Amplification.” Proc Natl Acad Sci USA 99(8): 5261-6.
40 Denda, M., Inoue, K., Inomata, S. and Denda, S. (2002). “Gamma-Aminobutyric Acid (a) Receptor Agonists Accelerate Cutaneous Barrier Recovery and Prevent Epidermal Hyperplasia Induced by Barrier Disruption.” J Invest Dermatol 119(5): 1041-7.
41 Dienz, O., Moller, A., Strecker, A., Stephan, N., Krammer, P. H., Droge, W. and Schmitz, M. L. (2003). “Src Homology 2 Domain-Containing Leukocyte Phosphoprotein of 76 Kda and Phospholipase C Gamma 1 Are Required for Nf-Kappa B Activation and Lipid Raft Recruitment of Protein Kinase C Theta Induced by T Cell Costimulation.” J Immunol 170(1): 365-72.
42 Dillon, N. (1993). “Regulating Gene Expression in Gene Therapy.” Trends Biotechnol 11(5): 167-73.
43 Drechsler, Y., Chavan, S., Catalano, D., Mandrekar, P. and Szabo, G. (2002). “Fcgammar Cross-Linking Mediates Nf-Kappab Activation, Reduced Antigen Presentation Capacity, and Decreased Il-12 Production in Monocytes without Modulation of Myeloid Dendritic Cell Development.” J Leukoc Biol 72(4): 657-67.
44 Drmanac, R., Drmanac, S., Chui, G., Diaz, R., Hou, A., Jin, H., Jin, P., Kwon, S., Lacy, S., Moeur, B., Shafto, J., Swanson, D., Ukrainczyk, T., Xu, C. and Little, D. (2002). “Sequencing by Hybridization (Sbh): Advantages, Achievements, and Opportunities.” Adv Biochem Eng Biotechnol 77: 75-101.
45 Elez, R., Piiper, A., Giannini, C. D., Brendel, M. and Zeuzem, S. (2000). “Polo-Like Kinase1, a New Target for Antisense Tumor Therapy.” Biochem Biophys Res Commun 269(2): 352-6.
46 Evans, B. E., Rittle, K. E., Bock, M. G., DiPardo, R. M., Freidinger, R. M., Whitter, W. L., Gould, N. P., Lundell, G. F., Homnick, C. F., Veber, D. F. and et al. (1987). “Design of Nonpeptidal Ligands for a Peptide Receptor: Cholecystokinin Antagonists.” J Med Chem 30(7): 1229-39.
47 Fackler, O. T. and Peterlin, B. M. (2000). “Endocytic Entry of Hiv-1.” Curr Biol 10(16): 1005-8.
48 Fakhrai-Rad, H., Zheng, J., Willis, T. D., Wong, K., Suyenaga, K, Moorhead, M., Eberle, J., Thorstenson, Y. R., Jones, T., Davis, R. W., Namsaraev, E. and Faham, M. (2004). “Snp Discovery in Pooled Samples with Mismatch Repair Detection.” Genome Res 14(7): 1404-12.
49 Fields, S. and Song, O. (1989). “A Novel Genetic System to Detect Protein-Protein Interactions.” Nature 340(6230): 245-6.
50 Fisher, G. J., Tavakkol, A., Leach, K., Burns, D., Basta, P., Loomis, C., Griffiths, C. E., Cooper, K. D., Reynolds, N. J., Elder, J. T. and et al. (1993). “Differential Expression of Protein Kinase C Isoenzymes in Normal and Psoriatic Adult Human Skin: Reduced Expression of Protein Kinase C-Beta Ii in Psoriasis.” J Invest Dermatol 101(4): 553-9.
51 Frank-Kamenetskii, M. D. and Mirkin, S. M. (1995). “Triplex DNA Structures.” Annu Rev Biochem 64: 65-95.
52 Fredriksson, L., Ehnman, M., Fieber, C. and Eriksson, U. (2005). “Structural Requirements for Activation of Latent Platelet-Derived Growth Factor Cc by Tissue Plasminogen Activator.” J Biol Chem 280(29): 26856-62.
53 Freeman, W. M., Walker, S. J. and Vrana, K. E. (1999). “Quantitative Rt-Pcr: Pitfalls and Potential.” Biotechniques 26(1): 112-22, 124-5.
54 Freshney, R. I., Ed. (1986). Animal Cell Culture: A Practical Approach. Oxford, IRL Press.
55 Freshney, R. I. (1987). Culture of Animal Cells: A Manual of Basic Technique. New York, A. R. Liss.
56 Fukuda, T., Chen, K., Shi, X. and Wu, C. (2003). “Pinch-1 Is an Obligate Partner of Integrin-Linked Kinase (Ilk) Functioning in Cell Shape Modulation, Motility, and Survival.” J Biol Chem 278(51): 51324-33. Epub 2003 Oct. 8.
57 Garoia, F., Grifoni, D., Trotta, V., Guerra, D., Pezzoli, M. C. and Cavicchi, S. (2005). “The Tumor Suppressor Gene Fat Modulates the Egfr-Mediated Proliferation Control in the Imaginal Tissues of Drosophila Melanogaster.” Mech Dev 122(2): 175-87.
58 Gasparini, P., Bonizzato, A., Dognini, M. and Pignatti, P. F. (1992). “Restriction Site Generating-Polymerase Chain Reaction (Rg-Pcr) for the Probeless Detection of Hidden Genetic Variation: Application to the Study of Some Common Cystic Fibrosis Mutations.” Mol Cell Probes 6(1): 1-7.
59 Gery, S. and Koeffler, H. P. (2003). “Repression of the Tmeff2 Promoter by C-Myc.” J Mol Biol 328(5): 977-83.
60 Gibbs, R. A., Nguyen, P. N. and Caskey, C. T. (1989). “Detection of Single DNA Base Differences by Competitive Oligonucleotide Priming.” Nucleic Acids Res 17(7): 2437-48.
61 Glover, D. M., Ed. (1985). DNA Cloning: A Practical Approach. Practical Approach Series. Oxford, Washington, D.C., IRL Press.
62 Gribskov, M. and Devereux, J., Eds. (1994). Sequence Analysis Primer. Uwbc Biotechnical Resource Series. New York, Stockton Press.
63 Griffin, A. M. and Griffin, H. G., Eds. (1994). Computer Analysis of Sequence Data. Part 1. Methods in Molecular Biology. Totowa, N.J., Humana Press.
64 Griffin, H. G. and Griffin, A. M. (1993). “DNA Sequencing. Recent Innovations and Future Trends.” Appl Biochem Biotechnol 38(1-2): 147-59.
65 Grossman, P. D., Bloch, W., Brinson, E., Chang, C. C., Eggerding, F. A., Fung, S., Iovannisci, D. M., Woo, S. and Winn-Deen, E. S. (1994). “High-Density Multiplex Detection of Nucleic Acid Sequences: Oligonucleotide Ligation Assay and Sequence-Coded Separation.” Nucleic Acids Res 22(21): 4527-34.
66 Guatelli, J. C., Whitfield, K. M., Kwoh, D. Y., Barringer, K. J., Richman, D. D. and Gingeras, T. R. (1990). “Isothermal, in vitro Amplification of Nucleic Acids by a Multienzyme Reaction Modeled after Retroviral Replication.” Proc Natl Acad Sci USA 87(5): 1874-8.
67 Hafner, G. J., Yang, I. C., Wolter, L. C., Stafford, M. R. and Giffard, P. M. (2001). “Isothermal Amplification and Multimerization of DNA by Bst DNA Polymerase.” Biotechniques 30(4): 852-6, 858, 860 passim.
68 Hamann, L., Kumpf, O., Muller, M., Visintin, A., Eckert, J., Schlag, P. M. and Schumann, R. R. (2004). “A Coding Mutation within the First Exon of the Human Md-2 Gene Results in Decreased Lipopolysaccharide-Induced Signaling.” Genes Immun 5(4): 283-8.
69 Haines, B. D. and Higgins, S. J., Eds. (1984). Transcription and Translation: A Practical Approach. Practical Approach Series. Oxford, IRL Press.
70 Hames, B. D. and Higgins, S. J., Eds. (1985). Nucleic Acid Hybridization: A Practical Approach. Practical Approach Series. Oxford, IRL Press.
71 Hanada, K., Kumagai, K., Yasuda, S., Miura, Y., Kawano, M., Fukasawa, M. and Nishijima, M. (2003). “Molecular Machinery for Non-Vesicular Trafficking of Ceramide.” Nature 426(6968): 803-9.
72 Hardenbol, P., Baner, J., Jain, M., Nilsson, M., Namsaraev, E. A., Karlin-Neumann, G. A., Fakhrai-Rad, H., Ronaghi, M., Willis, T. D., Landegren, U. and Davis, R. W. (2003). “Multiplexed Genotyping with Sequence-Tagged Molecular Inversion Probes.” Nat Biotechnol 21(6): 673-8.
73 Harlow, E. and Lane, D., Eds. (1988). Antibodies: A Laboratory Manual. New York, Cold Spring Harbor Laboratory.
74 Hayashi, K. (1992). “Pcr-Sscp: A Method for Detection of Mutations.” Genet Anal Tech Appl 9(3): 73-9.
75 Hayette, S., Gadoux, M., Martel, S., Bertrand, S., Tigaud, I., Magaud, J. P. and Rimokh, R. (1998). “Flrg (Follistatin-Related Gene), a New Target of Chromosomal Rearrangement in Malignant Blood Disorders.” Oncogene 16(22): 2949-54.
76 Heijne, G. V. (1987). Sequence Analysis in Molecular Biology: Treasure Trove or Trivial Pursuit. San Diego, Academic Press.
77 Hentges, S. T., Nishiyama, M., Overstreet, L. S., Stenzel-Poore, M., Williams, J. T. and Low, M. J. (2004). “Gaba Release from Proopiomelanocortin Neurons.” J Neurosci 24(7): 1578-83.
78 Hermonat, P. L. and Muzyczka, N. (1984). “Use of Adeno-Associated Virus as a Mammalian DNA Cloning Vector: Transduction of Neomycin Resistance into Mammalian Tissue Culture Cells.” Proc Natl Acad Sci USA 81(20): 6466-70.
79 Hiran, T. S., Mazurkiewicz, J. E., Kreienberg, P., Rice, F. L. and LaFlamme, S. E. (2003). “Endothelial Expression of the Alpha6beta4 Integrin Is Negatively Regulated During Angiogenesis.” J Cell Sci 116(Pt 18): 3771-81. Epub 2003 Aug. 5.
80 Hogan, B., Costantini, F. and Lacy, E., Eds. (1986). Manipulating the Mouse Embryo: A Laboratory Manual. New York, Cold Spring Harbor Laboratory.
81 Hogrefe, H. H., Mullinax, R. L., Lovejoy, A. E., Hay, B. N. and Sorge, J. A. (1993). “A Bacteriophage Lambda Vector for the Cloning and Expression of Immunoglobulin Fab Fragments on the Surface of Filamentous Phage.” Gene 128(1): 119-26.
82 Holm, S. J., Carlen, L. M., Mallbris, L., Stahle-Backdahl, M. and O'Brien, K. P. (2003). “Polymorphisms in the Seek1 and Spr1 Genes on 6p21.3 Associate with Psoriasis in the Swedish Population.” Exp Dermatol 12(4): 435-44.
83 Hoogendoorn, B., Norton, N., Kirov, G., Williams, N., Hamshere, M. L., Spurlock, G., Austin, J., Stephens, M. K., Buckland, P. R., Owen, M. J. and O'Donovan, M. C. (2000). “Cheap, Accurate and Rapid Allele Frequency Estimation of Single Nucleotide Polymorphisms by Primer Extension and Dhplc in DNA Pools.” Hum Genet 107(5): 488-93.
84 Hsu, I. C., Yang, Q., Kahng, M. W. and Xu, J. F. (1994). “Detection of DNA Point Mutations with DNA Mismatch Repair Enzymes.” Carcinogenesis 15(8): 1657-62.
85 Huber, B. E. and Carr, B. I., Eds. (1994). Molecular and Immunologic Approaches. Cancer Therapy in the Twenty-First Century. Mt. Kisco, N.Y., Futura Publishing Co.
86 Ishiguro, T., Nagawa, H., Naito, M. and Tsuruo, T. (2000). “Inhibitory Effect of Atf3 Antisense Oligonucleotide on Ectopic Growth of Ht29 Human Colon Cancer Cells.” Jpn J Cancer Res 91(8): 833-6.
87 Jinnin, M., Ihn, H., Mimura, Y., Asano, Y., Yamane, K. and Tamaki, K. (2005). “Regulation of Fibrogenic/Fibrolytic Genes by Platelet-Derived Growth Factor C, a Novel Growth Factor, in Human Dermal Fibroblasts.” J Cell Physiol. 202(2): 510-7.
88 Jobs, M., Howell, W. M., Stromqvist, L., Mayr, T. and Brookes, A. J. (2003). “Dash-2: Flexible, Low-Cost, and High-Throughput Snp Genotyping by Dynamic Allele-Specific Hybridization on Membrane Arrays.” Genome Res 13(5): 916-24.
89 Jones, D. A., Yawalkar, N., Suh, K. Y., Sadat, S., Rich, B. and Kupper, T. S. (2004). “Identification of Autoantigens in Psoriatic Plaques Using Expression Cloning.” J Invest Dermatol 123(1): 93-100.
90 Jones, J. (1992). Amino Acid and Peptide Synthesis. New York, Oxford University Press.
91 Jones, J. M. and Gellert, M. (2003). “Autoubiquitylation of the V(D)J Recombinase Protein Rag1.” Proc Natl Acad Sci USA 100(26): 15446-51. Epub 2003 Dec. 11.
92 Kanazawa, N., Kurosaki, M., Sakamoto, N., Enomoto, N., Itsui, Y., Yamashiro, T., Tanabe, Y., Maekawa, S., Nakagawa, M., Chen, C. H., Kakinuma, S., Oshima, S., Nakamura, T., Kato, T., Wakita, T. and Watanabe, M. (2004). “Regulation of Hepatitis C Virus Replication by Interferon Regulatory Factor 1.” J Virol 78(18): 9713-20.
93 Kanehisa, M. (1984). “Use of Statistical Criteria for Screening Potential Homologies in Nucleic Acid Sequences.” Nucleic Acids Res 12(1 Pt 1): 203-13.
94 Katagiri, K., Shimonaka, M. and Kinashi, T. (2004). “Rap1-Mediated Lymphocyte Function-Associated Antigen-1 Activation by the T Cell Antigen Receptor Is Dependent on Phospholipase C-Gamma1.” J Biol Chem 279(12): 11875-81.
95 Kataoka, T., Holler, N., Micheau, O., Martinon, F., Tinel, A., Hofmann, K. and Tschopp, J. (2001). “Bcl-Rambo, a Novel Bcl-2 Homologue That Induces Apoptosis Via Its Unique C-Terminal Extension.” J Biol Chem 276(22): 19548-54. Epub 2001 Mar. 21.
96 Kaufman, P. B., William, W. and Donghern, K., Eds. (1995). Handbook of Molecular and Cellular Methods in Biology and Medicine. Boca Raton, CRC Press.
97 Kettner, A., Kumar, L., Anton, I. M., Sasahara, Y., de la Fuente, M., Pivniouk, V. I., Falet, H., Hartwig, J. H. and Geha, R. S. (2004). “Wip Regulates Signaling Via the High Affinity Receptor for Immunoglobulin E in Mast Cells.” J Exp Med 199(3): 357-68.
98 Kim, H. J. and Kim, T. Y. (2005). “Regulation of Vascular Endothelial Growth Factor Expression by Insulin-Like Growth Factor-Ii in Human Keratinocytes, Differential Involvement of Mitogen-Activated Protein Kinases and Feedback Inhibition of Protein Kinase C.” Br J Dermatol 152(3): 418-25.
99 Kohler, G. and Milstein, C. (1992). “Continuous Cultures of Fused Cells Secreting Antibody of Predefined Specificity. 1975.” Biotechnology 24: 524-6.
100 Kotin, R. M. (1994). “Prospects for the Use of Adeno-Associated Virus as a Vector for Human Gene Therapy.” Hum Gene Ther 5(7): 793-801.
101 Kozal, M. J., Shah, N., Shen, N., Yang, R., Fucini, R., Merigan, T. C., Richman, D. D., Morris, D., Hubbell, E., Chee, M. and Gingeras, T. R. (1996). “Extensive Polymorphisms Observed in Hiv-1 Clade B Protease Gene Using High-Density Oligonucleotide Arrays.” Nat Med 2(7): 753-9.
102 Krane, J. F., Murphy, D. P., Gottlieb, A. B., Carter, D. M., Hart, C. E. and Krueger, J. G. (1991). “Increased Dermal Expression of Platelet-Derived Growth Factor Receptors in Growth-Activated Skin Wounds and Psoriasis.” J Invest Dermatol 96(6): 983-6.
103 Krebs, S., Seichter, D. and Forster, M. (2001). “Genotyping of Dinucleotide Tandem Repeats by Maldi Mass Spectrometry of Ribozyme-Cleaved Rna Transcripts.” Nat Biotechnol 19(9): 877-80.
104 Kremer, E. J. and Perricaudet, M. (1995). “Adenovirus and Adeno-Associated Virus Mediated Gene Transfer.” Br Med Bull 51(1): 31-44.
105 Kulinski, J., Besack, D., Oleykowski, C. A., Godwin, A. K. and Yeung, A. T. (2000). “Cel I Enzymatic Mutation Detection Assay.” Biotechniques 29(1): 44-6, 48.
106 Kurschat, P., Bielenberg, D., Rossignol-Tallandier, M., Stahl, A. and Klagsbrun, M. (2006). “Neuron Restrictive Silencer Factor Nrsf/Rest Is a Transcriptional Repressor of Neuropilin-1 and Diminishes the Ability of Semaphorin 3a to Inhibit Keratinocyte Migration.” J Biol Chem 281(5): 2721-9.
107 La Vecchia, C., Negri, E., Parazzini, F., Boyle, P., D'Avanzo, B., Levi, F., Gentile, A. and Franceschi, S. (1990). “Height and Cancer Risk in a Network of Case-Control Studies from Northern Italy.” Int J Cancer 45(2): 275-9.
108 Leitges, M., Mayr, M., Braun, U., Mayr, U., Li, C., Pfister, G., Ghaffari-Tabrizi, N., Baier, G., Hu, Y. and Xu, Q. (2001). “Exacerbated Vein Graft Arteriosclerosis in Protein Kinase Cdelta-Null Mice.” J Clin Invest 108(10): 1505-12.
109 Lesk, A. M., Ed. (1988). Computational Molecular Biology: Sources and Methods for Sequence Analysis. New York, Oxford University Press.
110 Livak, K. J., Marmaro, J. and Todd, J. A. (1995). “Towards Fully Automated Genome-Wide Polymorphism Screening.” Nat Genet 9(4): 341-2.
111 Lizardi, P. M., Huang, X., Zhu, Z., Bray-Ward, P., Thomas, D. C. and Ward, D. C. (1998). “Mutation Detection and Single-Molecule Counting Using Isothermal Rolling-Circle Amplification.” Nat Genet 19(3): 225-32.
112 Male, D., Cooke, A., Owen, M., Trowsdale, J. and Champion, B., Eds. (1996). Advanced Immunology. London, Baltimore, Mosby.
113 Mansfield, E. S., Vainer, M., Enad, S., Barker, D. L., Harris, D., Rappaport, E. and Fortina, P. (1996). “Sensitivity, Reproducibility, and Accuracy in Short Tandem Repeat Genotyping Using Capillary Array Electrophoresis.” Genome Res 6(9): 893-903.
114 Mao-Qiang, M., Fowler, A. J., Schmuth, M., Lau, P., Chang, S., Brown, B. E., Moser, A. H., Michalik, L., Desvergne, B., Wahli, W., Li, M., Metzger, D., Chambon, P. H., Elias, P. M. and Feingold, K. R. (2004). “Peroxisome-Proliferator-Activated Receptor (Ppar)-Gamma Activation Stimulates Keratinocyte Differentiation.” J Invest Dermatol 123(2): 305-12.
115 Marshall, D., Hardman, M. J., Nield, K. M. and Byrne, C. (2001). “Differentially Expressed Late Constituents of the Epidermal Cornified Envelope.” Proc Natl Acad Sci USA 98(23): 13031-6.
116 Martin-Vandelet, N., Paris, S., Bourguignon, J., Sesboue, R., Martin, J. P. and Diarra-Mehrpour, M. (1999). “Assembly and Secretion of Recombinant Chains of Human Inter-Alpha-Trypsin Inhibitor in Cos-7 Cells.” Eur J Biochem 259(1-2): 476-84.
117 Martin, F. C., Yeo, A. L. and Sonksen, P. H. (1997). “Growth Hormone Secretion in the Elderly: Aging and the Somatopause.” Baillieres Clin Endocrinol Metab 11(2): 223-50.
118 Martin, I., Muraglia, A., Campanile, G., Cancedda, R. and Quarto, R. (1997). “Fibroblast Growth Factor-2 Supports Ex vivo Expansion and Maintenance of Osteogenic Precursors from Human Bone Marrow.” Endocrinology 138(10): 4456-62.
119 Matera, L., Mori, M. and Galetto, A. (2001). “Effect of Prolactin on the Antigen Presenting Function of Monocyte-Derived Dendritic Cells.” Lupus 10(10): 728-34.
120 Matsuda, A., Suzuki, Y., Honda, G., Muramatsu, S., Matsuzaki, O., Nagano, Y., Doi, T., Shimotohno, K., Harada, T., Nishida, E., Hayashi, H. and Sugano, S. (2003). “Large-Scale Identification and Characterization of Human Genes That Activate Nf-Kappab and Mapk Signaling Pathways.” Oncogene 22(21): 3307-18.
121 Maxam, A. M. and Gilbert, W. (1977). “A New Method for Sequencing DNA.” Proc Natl Acad Sci USA 74(2): 560-4.
122 McPherson, M. J., Ed. (1991). Directed Mutagenesis: A Practical Approach. Practical Approach Series. New York, IRL Press.
123 McPherson, M. J., Quirke, P. and Taylor, G. R., Eds. (1991). Per, a Practical Approach. Practical Approach Series. New York, IRL Press.
124 Miccoli, L., Biard, D. S., Frouin, I., Harper, F., Maga, G. and Angulo, J. F. (2003). “Selective Interactions of Human Kin17 and Rpa Proteins with Chromatin and the Nuclear Matrix in a DNA Damage- and Cell Cycle-Regulated Manner.” Nucleic Acids Res 31(14): 4162-75.
125 Miccoli, L., Frouin, I., Novae, O., Di Paola, D., Harper, F., Zannis-Hadjopoulos, M., Maga, G., Biard, D. S. and Angulo, J. F. (2005). “The Human Stress-Activated Protein Kin17 Belongs to the Multiprotein DNA Replication Complex and Associates in vivo with Mammalian Replication Origins.” Mol Cell Biol 25(9): 3814-30.
126 Miller, A. D. (1992). “Human Gene Therapy Comes of Age.” Nature 357(6378): 455-60.
127 Miller, A. D., Garcia, J. V., von Suhr, N., Lynch, C. M., Wilson, C. and Eiden, M. V. (1991). “Construction and Properties of Retrovirus Packaging Cells Based on Gibbon Ape Leukemia Virus.” J Virol 65(5): 2220-4.
128 Miller, J. H. and Calos, M. P., Eds. (1987). Gene Transfer Vectors for Mammalian Cells. Current Communications in Molecular Biology. New York, Cold Spring Harbor Laboratory.
129 Mitani, K. and Caskey, C. T. (1993). “Delivering Therapeutic Genes—Matching Approach and Application.” Trends Biotechnol 11(5): 162-6.
130 Montanari, M., Boninsegna, A., Faraglia, B., Coco, C., Giordano, A., Cittadini, A. and Sgambato, A. (2005). “Increased Expression of Geminin Stimulates the Growth of Mammary Epithelial Cells and Is a Frequent Event in Human Tumors.” J Cell Physiol 202(1): 215-22.
131 Muramatsu, T. and Miyauchi, T. (2003). “Basigin (Cd147): A Multifunctional Transmembrane Protein Involved in Reproduction, Neural Function, Inflammation and Tumor Invasion.” Histol Histopathol 18(3): 981-7.
132 Muzyczka, N. (1994). “Adeno-Associated Virus (Aav) Vectors: Will They Work?” J Clin Invest 94(4): 1351.
133 Myers, R. M., Larin, Z. and Maniatis, T. (1985). “Detection of Single Base Substitutions by Ribonuclease Cleavage at Mismatches in Rna:DNA Duplexes.” Science 230(4731): 1242-6.
134 Myers, R. M., Lumelsky, N., Lerman, L. S. and Maniatis, T. (1985). “Detection of Single Base Substitutions in Total Genomic DNA.” Nature 313(6002): 495-8.
135 Nabel, G. J. and Felgner, P. L. (1993). “Direct Gene Transfer for Immunotherapy and Immunization.” Trends Biotechnol 11(5): 211-5.
136 Nagel, J. E., Smith, R. J., Shaw, L., Bertak, D., Dixit, V. D., Schaffer, E. M. and Taub, D. D. (2004). “Identification of Genes Differentially Expressed in T Cells Following Stimulation with the Chemokines Cxcl12 and Cxcl10.” BMC Immunol 5(1): 17.
137 Nagley, P. and Wei, Y. H. (1998). “Aging and Mammalian Mitochondrial Genetics.” Trends Genet 14(12): 513-7.
138 Nathan, C. and Shiloh, M. U. (2000). “Reactive Oxygen and Nitrogen Intermediates in the Relationship between Mammalian Hosts and Microbial Pathogens.” Proc Natl Acad Sci USA 97(16): 8841-8.
139 Nishihara, H., Maeda, M., Oda, A., Tsuda, M., Sawa, H., Nagashima, K. and Tanaka, S. (2002). “Dock2 Associates with Crk1 and Regulates Rac1 in Human Leukemia Cell Lines.” Blood 100(12): 3968-74. Epub 2002 Jun. 28.
140 Nishihara, H., Maeda, M., Tsuda, M., Makino, Y., Sawa, H., Nagashima, K. and Tanaka, S. (2002). “Dock2 Mediates T Cell Receptor-Induced Activation of Rac2 and Il-2 Transcription.” Biochem Biophys Res Commun 296(3): 716-20.
141 Niu, T., Qin, Z. S., Xu, X. and Liu, J. S. (2002). “Bayesian Haplotype Inference for Multiple Linked Single-Nucleotide Polymorphisms.” Am J Hum Genet 70(1): 157-69.
142 Noblesse, E., Cenizo, V., Bouez, C., Borel, A., Gleyzal, C., Peyrol, S., Jacob, M. P., Sommer, P. and Damour, O. (2004). “Lysyl Oxidase-Like and Lysyl Oxidase Are Present in the Dermis and Epidermis of a Skin Equivalent and in Human Skin and Are Associated to Elastic Fibers.” J Invest Dermatol 122(3): 621-30.
143 Nykvist, P., Tu, H., Ivaska, J., Kapyla, J., Pihlajaniemi, T. and Heino, J. (2000). “Distinct Recognition of Collagen Subtypes by Alpha(1)Beta(1) and Alpha(2)Beta(1) Integrins. Alpha(1)Beta(1) Mediates Cell Adhesion to Type Xiii Collagen.” J Biol Chem 275(11): 8255-61.
144 Oike, Y., Yasunaga, K., Ito, Y., Matsumoto, S., Maekawa, H., Morisada, T., Arai, F., Nakagata, N., Takeya, M., Masuho, Y. and Suda, T. (2003). “Angiopoietin-Related Growth Factor (Agf) Promotes Epidermal Proliferation, Remodeling, and Regeneration.” Proc Natl Acad Sci USA 100(16): 9494-9. Epub 2003 Jul. 18.
145 Orita, M., Iwahana, H., Kanazawa, H., Hayashi, K. and Sekiya, T. (1989). “Detection of Polymorphisms of Human DNA by Gel Electrophoresis as Single-Strand Conformation Polymorphisms.” Proc Natl Acad Sci USA 86(8): 2766-70.
146 Pakkanen, T. M., Laitinen, M., Hippelainen, M., Kallionpaa, H., Lehtolainen, P., Leppanen, P., Luoma, J. S., Tarvainen, R., Alhava, E. and Yla-Herttuala, S. (1999). “Enhanced Plasma Cholesterol Lowering Effect of Retrovirus-Mediated Ldl Receptor Gene Transfer to Whhl Rabbit Liver after Improved Surgical Technique and Stimulation of Hepatocyte Proliferation by Combined Partial Liver Resection and Thymidine Kinase—Ganciclovir Treatment.” Gene Ther 6(1): 34-41.
147 Paus, R. (1991). “Does Prolactin Play a Role in Skin Biology and Pathology?” Med Hypotheses 36(1): 33-42.
148 Pawlowitzki, I. H., Edwards, J. H. and Thompson, E. A., Eds. (1997). Genetic Mapping of Disease Genes. San Diego, Academic Press.
149 Perbal, B. V. (1984). A Practical Guide to Molecular Cloning. New York, Wiley.
150 Permutt, M. A., Wasson, J. C., Suarez, B. K., Lin, J., Thomas, J., Meyer, J., Lewitzky, S., Rennich, J. S., Parker, A., DuPrat, L., Maruti, S., Chayen, S. and Glaser, B. (2001). “A Genome Scan for Type 2 Diabetes Susceptibility Loci in a Genetically Isolated Population.” Diabetes 50(3): 681-5.
151 Peruski, L. F. and Peruski, A. H. (1997). The Internet and the New Biology. Tools for Genomic and Molecular Research. Washington, D.C., ASM Press.
152 Piepkorn, M., Pittelkow, M. R. and Cook, P. W. (1998). “Autocrine Regulation of Keratinocytes: The Emerging Role of Heparin-Binding, Epidermal Growth Factor-Related Growth Factors.” J Invest Dermatol 111(5): 715-21.
153 Prescott, J. E., Osthus, R. C., Lee, L. A., Lewis, B. C., Shim, H., Barrett, J. F., Guo, Q., Hawkins, A. L., Griffin, C. A. and Dang, C. V. (2001). “A Novel C-Myc-Responsive Gene, Jpo1, Participates in Neoplastic Transformation.” J Biol Chem 276(51): 48276-84. Epub 2001 Oct. 11.
154 Raap, A. K. (1998). “Advances in Fluorescence in situ Hybridization.” Mutat Res 400(1-2): 287-98.
155 Raghavachari, N., Krysan, K., Xing, K. and Lou, M. F. (2001). “Regulation of Thioltransferase Expression in Human Lens Epithelial Cells.” Invest Ophthalmol Vis Sci 42(5): 1002-8.
156 Ratcliffe, K. E., Tao, Q., Yavuz, B., Stoletov, K. V., Spring, S. C. and Terman, B. I. (2002). “Sck Is Expressed in Endothelial Cells and Participates in Vascular Endothelial Growth Factor-Induced Signaling.” Oncogene 21(41): 6307-16.
157 Raya, A., Revert-Ros, F., Martinez-Martinez, P., Navarro, S., Rosello, E., Vieites, B., Granero, F., Forteza, J. and Saus, J. (2000). “Goodpasture Antigen-Binding Protein, the Kinase That Phosphorylates the Goodpasture Antigen, Is an Alternatively Spliced Variant Implicated in Autoimmune Pathogenesis.” J Biol Chem 275(51): 40392-9.
158 Remick, D. G. (1995). “Applied Molecular Biology of Sepsis.” J Crit Care 10(4): 198-212.
159 Ronaghi, M., Uhlen, M. and Nyren, P. (1998). “A Sequencing Method Based on Real-Time Pyrophosphate.” Science 281(5375): 363, 365.
160 Rosenecker, J., Harms, K. H., Bertele, R. M., Pohl-Koppe, A., v Mutius, E., Adam, D. and Nicolai, T. (1996). “Adenovirus Infection in Cystic Fibrosis Patients: Implications for the Use of Adenoviral Vectors for Gene Transfer.” Infection 24(1): 5-8.
161 Ruiz-Velasco, N., Guerrero-Esteo, M., Briskin, M. J. and Teixido, J. (2000). “The Alpha(4) Integrin Subunit Tyr(187) Has a Key Role in Alpha(4)Beta(7)-Dependent Cell Adhesion.” J Biol Chem 275(10): 7052-9.
162 Saiki, R. K., Bugawan, T. L., Horn, G. T., Mullis, K. B. and Erlich, H. A. (1986). “Analysis of Enzymatically Amplified Beta-Globin and Hla-Dq Alpha DNA with Allele-Specific Oligonucleotide Probes.” Nature 324(6093): 163-6.
163 Saiki, R. K., Walsh, P. S., Levenson, C. H. and Erlich, H. A. (1989). “Genetic Analysis of Amplified DNA with Immobilized Sequence-Specific Oligonucleotide Probes.” Proc Natl Acad Sci USA 86(16): 6230-4.
164 Saleeba, J. A. and Cotton, R. G. (1993). “Chemical Cleavage of Mismatch to Detect Mutations.” Methods Enzymol 217: 286-95.
165 Sambrook, J., Fritsch, E. F. and Maniatis, T. (1989). Molecular Cloning: A Laboratory Manual. New York, Cold Spring Harbor Laboratory.
166 Samulski, R. J., Chang, L. S. and Shenk, T. (1989). “Helper-Free Stocks of Recombinant Adeno-Associated Viruses: Normal Integration Does Not Require Viral Gene Expression.” J Virol 63(9): 3822-8.
167 Sanger, F., Nicklen, S. and Coulson, A. R. (1977). “DNA Sequencing with Chain-Terminating Inhibitors.” Proc Natl Acad Sci USA 74(12): 5463-7.
168 Sato, C. (2003). “[Frequent Occurrence and Biological Significance of Degree of Polymerization in Sialic Acid].” Seikagaku 75(12): 1526-30.
169 Sauter, E. R., Herlyn, M., Liu, S. C., Litwin, S. and Ridge, J. A. (2000). “Prolonged Response to Antisense Cyclin D1 in a Human Squamous Cancer Xenograft Model.” Clin Cancer Res 6(2): 654-60.
170 Savoy, D. N., Billadeau, D. D. and Leibson, P. J. (2000). “Cutting Edge: Wip, a Binding Partner for Wiskott-Aldrich Syndrome Protein, Cooperates with Vav in the Regulation of T Cell Activation.” J Immunol 164(6): 2866-70.
171 Sayama, K., Hanakawa, Y., Shirakata, Y., Yamasaki, K., Sawada, Y., Sun, L., Yamanishi, K., Ichijo, H. and Hashimoto, K. (2001). “Apoptosis Signal-Regulating Kinase 1 (Ask1) Is an Intracellular Inducer of Keratinocyte Differentiation.” J Biol Chem 276(2): 999-1004.
172 Schottelius, A. J., Moldawer, L. L., Dinarello, C. A., Asadullah, K., Sterry, W. and Edwards, C. K., 3rd (2004). “Biology of Tumor Necrosis Factor-Alpha-Implications for Psoriasis.” Exp Dermatol 13(4): 193-222.
173 Sell, S. (1996). Immunology, Immunopathology and Immunity. Stamford, Conn., Appleton and Lange.
174 Shilatifard, A., Duan, D. R., Hague, D., Florence, C., Schubach, W. H., Conaway, J. W. and Conaway, R. C. (1997). “Ell2, a New Member of an Ell Family of Rna Polymerase Ii Elongation Factors.” Proc Natl Acad Sci USA 94(8): 3639-43.
175 Simon, H., Fortsch, I., Burckhardt, G., Gabrielyan, A., Birch-Hirschfeld, E., Stelzner, A., Prevot-Halter, I., Leumann, C. and Zimmer, C. (1999). “Triple Helix Formation Inhibits DNA Gyrase Activity.” Antisense Nucleic Acid Drug Dev 9(6): 527-31.
176 Skurk, T., van Harmelen, V. and Hauner, H. (2004). “Angiotensin Ii Stimulates the Release of Interleukin-6 and Interleukin-8 from Cultured Human Adipocytes by Activation of Nf-Kappab.” Arterioscler Thromb Vasc Biol 24(7): 1199-203. Epub 2004 May 6.
177 Smith, D. W., Ed. (1993). Biocomputing. Informatics and Genome Projects. New York, Academic Press.
178 Sommerfelt, M. A. and Weiss, R. A. (1990). “Receptor Interference Groups of 20 Retroviruses Plating on Human Cells.” Virology 176(1): 58-69.
179 Sterman, D. H., Treat, J., Litzky, L. A., Amin, K. M., Coonrod, L., Molnar-Kimber, K., Recio, A., Knox, L., Wilson, J. M., Albelda, S. M. and Kaiser, L. R. (1998). “Adenovirus-Mediated Herpes Simplex Virus Thymidine Kinase/Ganciclovir Gene Therapy in Patients with Localized Malignancy: Results of a Phase I Clinical Trial in Malignant Mesothelioma.” Hum Gene Ther 9(7): 1083-92.
180 Stites, D. P. and Terr, A. I., Eds. (1991). Basic and Clinical Immunology. Norwalk, Conn., Appleton and Lange.
181 Storm, N., Darnhofer-Patel, B., van den Boom, D. and Rodi, C. P. (2003). “Maldi-Tof Mass Spectrometry-Based Snp Genotyping.” Methods Mol Biol 212: 241-62.
182 Suzuki, Y., Miura, H., Tanemura, A., Kobayashi, K., Kondoh, G., Sano, S., Ozawa, K., Inui, S., Nakata, A., Takagi, T., Tohyama, M., Yoshikawa, K. and Itami, S. (2002). “Targeted Disruption of Lig-1 Gene Results in Psoriasiform Epidermal Hyperplasia.” FEBS Lett 521(1-3): 67-71.
183 Takeda, H., Katagata, Y., Hozumi, Y. and Kondo, S. (2004). “Effects of Angiotensin Ii Receptor Signaling During Skin Wound Healing.” Am J Pathol 165(5): 1653-62.
184 Takeichi, M. (1993). “Cadherins in Cancer: Implications for Invasion and Metastasis.” Curr Opin Cell Biol 5(5): 806-11.
185 Taniguchi, Y., Komatsu, N. and Moriuchi, T. (1995). “Overexpression of the Hox4a (Hoxd3) Homeobox Gene in Human Erythroleukemia Hel Cells Results in Altered Adhesive Properties.” Blood 85(10): 2786-94.
186 Teber, O. A., Gillessen-Kaesbach, G., Fischer, S., Bohringer, S., Albrecht, B., Albert, A., Arslan-Kirchner, M., Haan, E., Hagedorn-Greiwe, M., Hammans, C., Henn, W., Hinkel, G. K., Konig, R., Kunstmann, E., Kunze, J., Neumann, L. M., Prott, E. C., Rauch, A., Rott, H. D., Seidel, H., Spranger, S., Sprengel, M., Zoll, B., Lohmann, D. R. and Wieczorek, D. (2004). “Genotyping in 46 Patients with Tentative Diagnosis of Treacher Collins Syndrome Revealed Unexpected Phenotypic Variation.” Eur J Hum Genet 12(11): 879-90.
187 Tenaud, I., Leroy, S., Chebassier, N. and Dreno, B. (2002). “Modulation in vitro of Keratinocyte Integrins by Interferon-Alpha and Interferon-Gamma.” Int J Dermatol 41(12): 836-40.
188 Tordjman, R., Lepelletier, Y., Lemarchandel, V., Cambot, M., Gaulard, P., Hermine, O. and Romeo, P. H. (2002). “A Neuronal Receptor, Neuropilin-1, Is Essential for the Initiation of the Primary Immune Response.” Nat Immunol 3(5): 477-82.
189 Tornillo, L., Carafa, V., Richter, J., Sauter, G., Moch, H., Minola, E., Gambacorta, M., Bianchi, L., Vecchione, R. and Terracciano, L. M. (2000). “Marked Genetic Similarities between Hepatitis B Virus-Positive and Hepatitis C Virus-Positive Hepatocellular Carcinomas.” J Pathol 192(3): 307-12.
190 Tratschin, J. D., West, M. H., Sandbank, T. and Carter, B. J. (1984). “A Human Parvovirus, Adeno-Associated Virus, as a Eucaryotic Vector: Transient Expression and Encapsidation of the Procaryotic Gene for Chloramphenicol Acetyltransferase.” Mol Cell Biol 4(10): 2072-81.
191 Tsuruoka, H., Khovidhunkit, W., Brown, B. E., Fluhr, J. W., Elias, P. M. and Feingold, K. R. (2002). “Scavenger Receptor Class B Type I Is Expressed in Cultured Keratinocytes and Epidermis. Regulation in Response to Changes in Cholesterol Homeostasis and Barrier Requirements.” J Biol Chem 277(4): 2916-22.
192 Uhart, M., McCaul, M. E., Oswald, L. M., Choi, L. and Wand, G. S. (2004). “Gabra6 Gene Polymorphism and an Attenuated Stress Response.” Mol Psychiatry 9(11): 998-1006.
193 Vaahtoranta-Lehtonen, H. H., Lehtonen, O. P., Harvima, I., Peltola, O. and Nikoskelainen, E. (1999). “Papillary Hypertrophy of the Upper Tarsal Conjunctiva During Contact Lens Wear: A 4-Month Study with Ethyl-6-O-Decanoyl-Glucoside.” Clao J 25(2): 105-8.
194 Vaccaro, M., Magaudda, L., Cutroneo, G., Trimarchi, F., Barbuzza, O., Guarneri, F. and Guarneri, B. (2002). “Changes in the Distribution of Laminin Alpha1 Chain in Psoriatic Skin: Immunohistochemical Study Using Confocal Laser Scanning Microscopy.” Br J Dermatol 146(3): 392-8.
195 Van Den Eynde, B. J., Gaugler, B., Probst-Kepper, M., Michaux, L., Devuyst, O., Lorge, F., Weynants, P. and Boon, T. (1999). “A New Antigen Recognized by Cytolytic T Lymphocytes on a Human Kidney Tumor Results from Reverse Strand Transcription.” J Exp Med 190(12): 1793-800.
196 Walker, J. M. (1998). “Pathomechanics and Classification of Cartilage Lesions, Facilitation of Repair.” J Orthop Sports Phys Ther 28(4): 216-31.
197 Walker, J. M. (1998). “Protein Protocols on Cd-Rom.”
198 Wankell, M., Munz, B., Hubner, G., Hans, W., Wolf, E., Goppelt, A. and Werner, S. (2001). “Impaired Wound Healing in Transgenic Mice Overexpressing the Activin Antagonist Follistatin in the Epidermis.” Embo J 20(19): 5361-72.
199 Warton, K., Foster, N. C., Gold, W. A. and Stanley, K. K. (2004). “A Novel Gene Family Induced by Acute Inflammation in Endothelial Cells.” Gene 342(1): 85-95.
200 Watson, K. L., Justice, R. W. and Bryant, P. J. (1994). “Drosophila in Cancer Research: The First Fifty Tumor Suppressor Genes.” J Cell Sci Suppl 18: 19-33.
201 Weir, D. M., Herzenberg, L. A., Blackwell, C. and Herzenberg, L. A., Eds. (1986). Handbook of Experimental Immunology. Oxford, England, Blackwell Scientific Publications.
202 Werner, S., Beer, H. D., Mauch, C., Luscher, B. and Werner, S. (2001). “The Mad1 Transcription Factor Is a Novel Target of Activin and Tgf-Beta Action in Keratinocytes: Possible Role of Mad1 in Wound Repair and Psoriasis.” Oncogene 20(51): 7494-504.
203 West, A. B., Lockhart, P. J., O'Farell, C. and Farrer, M. J. (2003). “Identification of a Novel Gene Linked to Parkin Via a Bi-Directional Promoter.” J Mol Biol 326(1): 11-9.
204 West, M. H., Trempe, J. P., Tratschin, J. D. and Carter, B. J. (1987). “Gene Expression in Adeno-Associated Virus Vectors: The Effects of Chimeric Mrna Structure, Helper Virus, and Adenovirus Va1 Rna.” Virology 160(1): 38-47.
205 Wilson, C., Reitz, M. S., Okayama, H. and Eiden, M. V. (1989). “Formation of Infectious Hybrid Virions with Gibbon Ape Leukemia Virus and Human T-Cell Leukemia Virus Retroviral Envelope Glycoproteins and the Gag and Pol Proteins of Moloney Murine Leukemia Virus.” J Virol 63(5): 2374-8.
206 Woodward, J., Ed. (1985). Immobilised Cells and Enzymes: A Practical Approach. Practical Approach Series. Oxford, England, IRL Press.
207 Wu, R., Ed. (1987). Methods in Enzymology. Recombinant DNA Part F. Amsterdam, Elsevier Science.
208 Wu, R. and Grossman, L., Eds. (1987). Methods in Enzymology. Recombinant DNA Part E. Amsterdam, Elsevier Science.
209 Xie, W., Li, F., Kudlow, J. E. and Wu, C. (1998). “Expression of the Integrin-Linked Kinase (Ilk) in Mouse Skin: Loss of Expression in Suprabasal Layers of the Epidermis and up-Regulation by Erbb-2.” Am J Pathol 153(2): 367-72.
210 Yajnik, V., Paulding, C., Sordella, R., McClatchey, A. I., Saito, M., Wahrer, D. C., Reynolds, P., Bell, D. W., Lake, R., van den Heuvel, S., Settleman, J. and Haber, D. A. (2003). “Dock4, a Gtpase Activator, Is Disrupted During Tumorigenesis.” Cell 112(5): 673-84.
211 Yan, C., Jamaluddin, M. S., Aggarwal, B., Myers, J. and Boyd, D. D. (2005). “Gene Expression Profiling Identifies Activating Transcription Factor 3 as a Novel Contributor to the Proapoptotic Effect of Curcumin.” Mol Cancer Ther 4(2): 233-41.
212 Yang, B., Wen, X., Kodali, N. S., Oleykowski, C. A., Miller, C. G., Kulinski, J., Besack, D., Yeung, J. A., Kowalski, D. and Yeung, A. T. (2000). “Purification, Cloning, and Characterization of the Cel I Nuclease.” Biochemistry 39(13): 3533-41.
213 Yin, X. Y., Grove, L. E. and Prochownik, E. V. (2001). “Mmip-2/Rnf-17 Enhances C-Myc Function and Regulates Some Target Genes in Common with Glucocorticoid Hormones.” Oncogene 20(23): 2908-17.
214 Yu, M., Poeschla, E. and Wong-Staal, F. (1994). “Progress Towards Gene Therapy for Hiv Infection.” Gene Ther 1(1): 13-26.
215 Zhang, C., Kawauchi, J., Adachi, M. T., Hashimoto, Y., Oshiro, S., Aso, T. and Kitajima, S. (2001). “Activation of Jnk and Transcriptional Repressor Atf3/Lrf1 through the Ire1/Traf2 Pathway Is Implicated in Human Vascular Endothelial Cell Death by Homocysteine.” Biochem Biophys Res Commun 289(3): 718-24.
216 Zhang, L., Koivisto, L., Heino, J. and Uitto, V. J. (2004). “Bacterial Heat Shock Protein 60 May Increase Epithelial Cell Migration through Activation of Map Kinases and Inhibition of Alpha6beta4 Integrin Expression.” Biochem Biophys Res Commun 319(4): 1088-95.
217 Zhou, X., Krueger, J. G., Kao, M. C., Lee, E., Du, F., Menter, A., Wong, W. H. and Bowcock, A. M. (2003). “Novel Mechanisms of T-Cell and Dendritic Cell Activation Revealed by Profiling of Psoriasis on the 63,100-Element Oligonucleotide Array.” Physiol Genomics 13(1): 69-78.

Books:

Abbas A K, Litchman A H. Cellular and Molecular Immunology. Philadelphia: Saunders; 1991. 417 p.

Austen B M and Westwood O M R. Protein Targeting and Secretion. Oxford: IRL Press; 1991. 85 p.

Bishop M J, editor. Guide to Human Genome Computing, 2d ed. San Diego: Academic Press; 1998. 306 p.

Cowell I G, Austin C A, editors. DNA Library Protocols. Methods in Molecular Biology. Vol. 69 Totowa, N.J.: Humana Press; 1997. 321 p.

Freshney R I, editor. Animal Cell Culture: A Practical Approach. Oxford: IRL Press; 1986.

Freshney R I. Culture Of Animal Cells: A Manual of Basic Technique. New York: A R Liss; 1987. 397 p.

Glover D M, editor. DNA Cloning: A Practical Approach. Vols 1 & 2. Oxford; Washington: IRL Press; 1985.

Gribskov M, Devereux J, editors. Sequence Analysis Primer. Oxford University Press; 1994. 296 p.

Griffin A M, Griffin H G, editors. ComputerAnalysis of Sequence Data, Part 1. Totowa, N.J. Humana Press; 1994. 392 p.

Hames B D, Higgins S J, editors. Nucleic Acid Hybridization: A Practical Approach. Oxford: IRL Press; 1985. 245 p.

Hames B D, Higgins S J, editors. Transcription and Translation: A Practical Approach. Oxford: IRL Press; 1984. 328 p.

Harlow Ed, Lane D. Antibodies: A Laboratory Manual. New York: Cold Spring Harbor Laboratory; 1988. 726 p.

Heinje G. von. Sequence Analysis in Molecular Biology. San Diego: Academic Press; 1987. 188 p.

Hogan B, Costantini F, Lacy E, editors. Manipulating the Mouse Embryo: A Laboratory Manual. New York: Cold Spring Harbor Laboratory Press; 1986. 332 p.

Huber B E, Can B I. Molecular and Immunologic Approaches. Mt. Kisco, N.Y.: Futura Publishing Co; 1994.

Jones J. Amino Acid and Peptide Synthesis. Oxford; New York: Oxford Science Publications; 1992. 86 p.

Kaufman P B, William W, Donghern K, editors. Handbook of Molecular and Cellular Methods in Biology and Medicine. Boca Raton: CRC Press; 1995. 484 p.

Lesk A M, editor. Computational Molecular Biology: sources and methods for sequence analysis. New York: Oxford University Press; 1988. 254 p.

Male D, Cooke A, Owen M, Trowsdale J, Champion B, editors. Advanced Immunology. 3rd ed. London; Baltimore: Mosby; 1996. 273 p.

McPherson M J, editor. Directed Mutagenesis: A Practical Approach. New York: IRL Press; 1991. 257 p.

McPherson M J, Quirke P, Taylor J R, editors. PCR: A Practical Approach. Oxford; New York: IRL Press; 1991. 253 p.

Miller J H, Calos M P, editors. Gene Transfer Vectors for Mammalian Cells. New York: Cold Spring Harbor Laboratory Press; 1987. 169 p.

Miller J H, Calos M P, editors. Gene Transfer Vectors For Mammalian Cells. New York: Cold Spring Harbor Laboratory; 1987. 169 p.

Pawlowitzki I H, Edwards J H, Thompson E A, editors. Genetic Mapping of Disease Genes. Academic Press London; 1997. 288 p.

Perbal B V. A Practical Guide to Molecular Cloning. 1st ed. New York: Wiley Interscience Publication; 1984. 554 p.

Perbal B V. A Practical Guide To Molecular Cloning. New York: Wiley; 1984. 554 p.

Peruski L F, Peruski A H. The Internet and the New Biology. Tools for Genomic and Molecular Research. Washington, D.C.: American Society for Microbiology Press; 1997.

Sambrook J. Molecular Cloning: A Laboratory Manual. 2nd ed. 3 vols. New York: Cold Spring Harbor Laboratory Press; 1989.

Sell S. Immunology, Immunopathology & Immunity. 5th ed. Stamford, Conn.: Appleton & Lange; 1996. 1014 p.

Smith D W, editor. Biocomputing. Informatics and Genome Projects, New York: Academic Press; 1993. 336 p.

Stites D P, Terr A T, editors. Basic and Clinical Immunology. 7th ed. Norwalk, Conn.: Appleton & Lange; 1991. 870 p.

Walker J M. Protein Protocols on CD-ROM, Humana Press, Totowa, N.J.

Weir D M, Herzenberg L A, Blackwell C, editors. Handbook Of Experimental Immunology. 4 vols. Oxford: Blackwell; 1986.

Woodward J. Immobilized Cells And Enzymes: A Practical Approach. Oxford: IRL Press; 1986.

Wu R, Grossman L, editors. Methods in Enzymology: Rexcombinant DNA Part E. Vol. 154. Amsterdam: Elsevier Science; 1987. 576 p.

Wu R, Grossman L, editors. Methods in Enzymology: Rexcombinant DNA Part F. Vol. 155. Amsterdam: Elsevier Science; 1987. 628 p.

Patent:

WO02090571A3

WO05016282A2

U.S. Pat. No. 4,683,195

U.S. Pat. No. 4,683,202.

U.S. Pat. No. 4,952,501.

WO03042661A2

U.S. 20040009479A1

U.S. Pat. No. 5,315,000

WO1997US0005216

U.S. Pat. No. 5,498,531

U.S. Pat. No. 5,807,718

U.S. Pat. No. 5,888,819

U.S. Pat. No. 6,090,543

U.S. Pat. No. 6,090,606

U.S. Pat. No. 5,585,089

U.S. Pat. No. 4,683,195

U.S. Pat. No. 4,683,202

U.S. Pat. No. 5,459,039.

U.S. Pat. No. 6,090,543).

U.S. Pat. No. 6,090,606

U.S. Pat. No. 5,869,242

U.S. Patent No. 60/335,068

U.S. Pat. No. 6,479,244

PCT/US94/05700

U.S. Pat. No. 4,797,368;

WO 93/24641

U.S. Pat. No. 5,173,414

We claim:

1. A method of constructing a psoriasis GeneMap in a human population, comprising screening for the expression level of or presence or absence of at least one allele of at least one gene from Tables 10-12 and 14 in at least one sample.

2. (canceled)

3. The method of claim 1, wherein said population is a founder population.

4. The method of claim 3, wherein said founder population is the population of Quebec.

5. A method of constructing a GeneMap in a human population, comprising screening for the presence or absence of at least one allele of at least one single nucleotide polymorphisms (SNPs) from Tables 2-9 and 13 in at least one sample.

6. (canceled)

7. The method of claim 5, wherein said population is a founder population.

8. The method of claim 7, wherein said founder population is the population of Quebec.

9. A method of genetic mapping for detecting the association of at least one marker for psoriasis comprising: a) obtaining biological samples from at least one disease patient; b) screening for the presence or absence of at least one allele of at least one SNP or a group of SNPs from Tables 2-9 and 13 within each biological sample; and c) evaluating whether said SNP or a group of SNPs shows a statistically significant skewed distribution between a group of patients compared to a group of controls.

10. The method of claim 9, wherein said biological samples are selected from the group consisting of fluid, serum, tissue swabs, buccal swabs, saliva, mucus, urine, stools, spermatozoids, vaginal secretions, lymph, amniotic liquid, pleural liquid and tears.

11. The method of claim 9, wherein said patients and controls are from a human population.

12. (canceled)

13. The method of claim 11, wherein said population is a founder population.

14. The method of claim 13, wherein said founder population is the population of Quebec.

15. (canceled)

16. The method of claim 9, wherein said patients and controls are recruited in the form of trios comprising two parents and one child or one parent and two children, or in the form of unrelated individuals.

17. The method of claim 9, wherein said screening is performed using an assay selected from the group consisting of an allele-specific hybridization assay, an oligonucleotide ligation assay, an allele-specific elongation/ligation assay, an allele-specific amplification assay, a single-base extension assay, a molecular inversion probe assay, an invasive cleavage assay, a selective termination assay, RPLP, sequencing assay, SSCP, mismatch-cleaving assay, and denaturing gradient gel electrophoresis.

18. The method of claim 16, wherein said trios are members of a cohort.

19. The method of claim 9, wherein said screening is carried out on pools of patients and pools of controls.

20. The method of claim 9, wherein the genotype distribution is compared one SNP at a time.

21. The method of claim 9, wherein a genotype distribution is compared with a group of markers from Tables 2-9 and 13 forming a haplotype.

22-26. (canceled)

27. A method for predicting the efficacy of a drug for treating psoriasis in a human patient, comprising: a) obtaining a sample of cells from the patient; b) obtaining a gene expression profile from the sample in the absence and presence of in vitro modulation of the cells with specific mediators; the gene expression profile comprising one or more genes from Tables 10-12 and 14; and c) comparing the gene expression profile of the sample with a reference gene expression profile, wherein similarity between the sample expression profile and the reference expression profile predicts the efficacy of the drug for treating psoriasis in the patient.

28. The method of claim 27, further comprising exposing the sample of cells to the drug for treating psoriasis prior to obtaining the gene expression profile of the sample.

29. The method of claim 27, wherein the sample of cells is derived from a tissue selected from the group consisting of: the scalp, blood, dermis, epidermis, skin, cutaneous surfaces, intertrigious areas, genitalia, vessels and endothelium.

30. The method of claim 29, wherein the cells are selected from the group consisting of: keratinocytes, monocytes, neutrophils, Langerhans cells, CD4+ and CD8+ T cells and lymphocytes.

31. The method of claim 27, wherein the sample is obtained via biopsy.

32. The method of claim 27, wherein the gene expression profile comprises expression values for at least two of the genes listed in Tables 10-12 and 14.

33. The method of claim 32, wherein the gene expression profile of the sample is obtained by detecting the protein products of said genes.

34. The method of claim 27, wherein the gene expression profile of the sample is obtained using a hybridization assay to oligonucleotides contained in a microarray.

35. The method of claim 34, wherein the oligonucleotides comprises nucleic acid molecules at least 95% identical to SEQ ID from Tables 2-14.

36. The method of claim 27, wherein the reference expression profile is that of cells derived from patients that do not have psoriasis.

37. The method of claim 27, wherein the drug is selected from the group consisting of symptom relievers and anti-inflammatory drugs for an inflammatory disease condition.

38. A method for inducing a psoriasis-like state in a resident tissue or cell, comprising contacting the tissue or cell with at least one gene from Tables 10-12 and 14 or modulating the expression of at least one gene from Tables 10-12 and 14 in the resident tissue or cell.

39. The method of claim 38, wherein the resident tissue or cell is derived from the group consisting of blood, dermis, epidermis, skin, cutaneous surfaces, intertrigious areas, genitalia, vessels and endothelium, keratinocytes, monocytes, neutrophils, Langerhans cells, CD4+ and CD8+ T cells and lymphocytes.

40. A method for screening drug candidates for treating psoriasis, comprising: a) contacting a resident cell induced by the method of claim 38 with a drug candidate for treating psoriasis; and b) assaying for a pro-inflammatory like state, such that an absence of the proinflammatory like state is indicative of the drug candidate being effective in treating psoriasis.

41-44. (canceled)

45. A drug screening assay comprising: a) administering one or more test compounds to an animal having psoriasis, or a cell composition isolated there from; and b) comparing the level of gene expression of at least one gene from Tables 10-12 and 14 in the presence of the one or more test compounds with one or both of the levels of said gene expression in the absence of the one or more test compounds or in normal cells; wherein test compounds which cause the level of expression of one or more genes from Tables 10-12 and 14 to approach normal are candidates for drugs to treat psoriasis.

46-47. (canceled)

48. A method for identifying a gene that regulates drug response in psoriasis, comprising: a) obtaining a gene expression profile for at least one gene from Tables 10-12 and 14 in a resident tissue cell induced to a pro-inflammatory like state in the presence of the candidate drug; and b) comparing the expression profile of said gene to a reference expression profile for said gene in a cell induced to the pro-inflammatory like state in the absence of the candidate drug, wherein genes whose expression is altered by the drug relative to the reference expression profile may identify the gene as a gene that regulates drug response in psoriasis.

49. An expression profile indicative of the presence of psoriasis in a patient, comprising an expression level of at least one gene of Tables 10-12 and 14.

50-52. (canceled)

53. A method of diagnosing susceptibility to psoriasis in an individual, comprising screening for an at-risk haplotype of at least one gene from Tables 10-12 and 14, or comprising at least one SNP from Tables 2-9 and 13, that is more frequently present in an individual susceptible to psoriasis, compared to the frequency of its presence in a control individual, wherein the presence of the at-risk haplotype is indicative of a susceptibility to psoriasis.

54. The method of claim 53, wherein the at-risk haplotype is characterized by the presence of at least one single nucleotide polymorphism from Tables 2-9 and 13.

55. The method of claim 53, wherein said screening comprises enzymatic amplification of nucleic acid from said individual or amplification using universal oligos on elongation/ligation products.

56. The method of claim 55, wherein the nucleic acid is DNA.

57. The method of claim 56, wherein the DNA is human DNA.

58. The method of claim 55, wherein said screening further comprises determining the presence or absence of an at-risk haplotype in said amplified nucleic acid.

59. The method of claim 58, wherein said determining is performed by an assay selected from the group consisting of electrophoretic analysis, restriction length polymorphism analysis, sequence analysis and hybridization analysis.

60-104. (canceled)

105. A method of assessing a patient's risk of having or developing psoriasis, comprising: a) determining the level of expression of at least one gene from Tables 4-6 or gene products thereof, and comparing the level of expression to a normal cell; and b) assessing a patient's risk of having or developing psoriasis, if any, by determining the correlation between the differential expression of said genes or gene products with known changes in expression of said genes measured in at least one patent suffering from psoriasis.

106-107. (canceled)

108. A method of diagnosing psoriasis in a patient, comprising detecting a nucleic acid molecule encoding at least one protein from Tables 10-12 and 14 in a fluid or tissue sample from the patient.

109. The method of claim 108, wherein the detection comprises detecting at least one associated allele, particular allele of a polymorphic locus, or the like in the nucleic acid molecule encoding said at least one protein.

110. The method of claim 109, wherein said method comprises hybridizing at least one probe to said sample under stringent conditions which allow hybridization of said at least one probe to nucleic acid comprising said associated allele, a particular allele of a polymorphic locus, or the like, wherein the presence of a hybridization signal indicates the presence of said associated allele, particular allele of a polymorphic locus, or the like, in at least one gene from Tables 10-12 and 14.

111. The method of claim 110, wherein the nucleic acid molecule has been amplified prior to said hybridization.

112. The method of claim 111, wherein said method comprises using a single-stranded conformation polymorphism technique to assay for detecting said associated allele, particular allele of a polymorphic locus, or the like.

113. The method of claim 111, wherein said method further comprises sequencing at least one gene from Tables 4-6 from the sample.

114. (canceled)

115. The method of claim 111, wherein said method comprises sequencing at least one gene from Tables 10-12 and 14 in the sample.

116. The method of claim 111, wherein said method comprises determining the sequence of at least one gene from Tables 10-12 and 14 by preparing a cDNA from a RNA taken from said sample and sequencing said cDNA to determine the presence or absence of an associated allele, a particular allele of a polymorphic locus, or the like.

117. The method of claim 110, wherein said method comprises performing an RNAse assay.

118. The method of claim 110, wherein said at least one probe is attached to a microarray or a bead.

119. The method of claim 110, wherein said at least one probe is an oligonucleotide.

120. (canceled)

121. The method of claim 109, wherein the associated allele, particular allele of a polymorphic locus, or the like is selected from the group consisting of at least one of the SNPs from Tables 2-9 and 13, alone or in combination.

122. A method for assaying the presence of a nucleic acid associated with resistance or susceptibility to psoriasis in a sample, comprising: contacting said sample with a nucleic acid recited in claim 1 under stringent hybridization conditions; and detecting the presence of a hybridization complex wherein the presence of the hybridization complex is indicative of the presence of a nucleic acid associated with resistance or susceptibility to psoriasis.

123. A method for diagnosing or determining psoriasis or the predisposition to psoriasis, or the progression of psoriasis, comprising obtaining a sample from a patient; contacting the sample with a nucleic acid of Tables 2-9 and 13; and detecting the presence or absence of a hybridization complex, wherein the presence or absence of a hybridization complex is a diagnosis of psoriasis.

124-128. (canceled)

129. A method for predicting the efficacy of a drug for treating psoriasis disease in a human patient, comprising: a) obtaining a sample of cells from the patient; b) obtaining a set of genotypes from the sample, wherein the set of genotypes comprises genotypes of one or more polymorphic loci from Tables 2-14; and c) comparing the set of genotypes of the sample with a set of genotypes associated with efficacy of the drug, wherein similarity between the set of genotypes of the sample and the set of genotypes associated with efficacy of the drug predicts the efficacy of the drug for treating psoriasis disease in the patient.

130. The method of claim 129, wherein the sample of cells is derived from a tissue selected from the group consisting of: the scalp, GI track, muscle, sebaceous gland, nerve, blood, dermis, epidermis, skin, cutaneous surfaces, intertrigious areas, genitalia, vessels and endothelium.

131. The method of claim 130, wherein the cells are selected from the group consisting of: blood, dermis, epidermis, skin, cutaneous surfaces, intertrigious areas, genitalia, vessels and endothelium, keratinocytes, monocytes, neutrophils, Langerhans cells, CD4+ and CD8+ T cells and lymphocytes.

132. The method of claim 129, wherein the sample is obtained via biopsy.

133. The method of claim 129, wherein the set of genotypes from the sample comprises genotypes of at least two of the polymorphic loci listed in Tables 2-14.

134. The method of claim 129 wherein the set of genotypes from the sample is obtained by hybridization to allele-specific oligonucleotides complementary to the polymorphic loci from Tables 2-14, wherein said allele-specific oligonucleotides are contained on a microarray.

135. The method of claim 134, wherein the oligonucleotides comprise nucleic acid molecules at least 95% identical to SEQ ID from Tables 2-14.

136. The method of claim 129, wherein the set of genotypes from the sample is obtained by sequencing said polymorphic loci in said sample.

137. The method of claim 129, wherein the drug is selected from the group consisting of symptom relievers and drugs for psoriasis disease.

138-148. (canceled)

149. A method of assessing a patient's risk of having or developing psoriasis disease, comprising: a) selecting at least one polymorphic locus from Tables 2-14; b) determining a genotype for at least one polymorphic locus from Tables 2-14 in a patient; c) comparing said genotype of b) to a genotype for at least one polymorphic locus from Tables 2-14 that is associated with psoriasis disease; and d) assessing the patient's risk of having or developing psoriasis disease, wherein said patient has a higher risk of having or developing psoriasis disease if the genotype for at least one polymorphic locus from Tables 2-14 in said patient is the same as said genotype for at least one polymorphic locus from Tables 2-14 that is associated with psoriasis disease.
